(19) United States
(12) Patent Application Publication
(10) Pub. No.: US 2010/0278846 A1
(43) Pub. Date: Nov. 4, 2010

(54) NASAL-ADMINISTERED VACCINES USING MULTI-SCREENED NALT-TARGETING AND PHAGOCYTIC POLYPEPTIDE TRANSPORT SEQUENCES

(76) Inventor: IAN A. FERGUSON, Belmont (AU)

Correspondence Address: PATRICK D. KELLY, 11939 MANCHESTER #403, ST. LOUIS, MO, 63131, US

(21) Appl. No.: 12/636,705

(22) Filed: Dec. 11, 2009

Related U.S. Application Data
(63) Continuation-in-part of application No. 11/571,241, filed on Nov. 23, 2007.

(30) Foreign Application Priority Data
Jun. 23, 2004 (AU) 2004/903,380

(51) Int. Cl. A61K 39/00; A61P 37/04

(52) U.S. Cl. 424/184.1

(57) ABSTRACT

Multiple sequential screening tests have been performed on phage display libraries, and polypeptide sequences have been identified that potently drive both: (i) intake into mucosal immune cells, including NALT cells in the nose and throat; and, (ii) phagocytic intake and processing by antigen-presenting cells, such as macrophages. Such polypeptide sequences can be used as potent “target and deliver” components in vaccines that can be administered nasally, or to other mucous membranes. Such vaccines can be made very rapidly and in huge quantities, from bacteriophages that will also carry antigenic sequences in their coat proteins, or other immunoactive components. Alternately, such “target and deliver” polypeptides can be incorporated into vaccines derived from eukaryotic viruses or cellular pathogens. Enhancements also are disclosed, such as agents that can activate one or more types of toll-like receptors, to increase immunes responses and guide them in desired directions.

### RELATED APPLICATIONS

This application is a continuation-in-part of U.S. patent application Ser. No. 11/571,241, filed Jun. 23, 2005 (section 371 date Nov. 23, 2007 based on PCT application PCT IB05/04077), which in turn claimed priority based on Australian patent application 2004/903,380, filed Jun. 23, 2004.

### BACKGROUND OF THE INVENTION

This invention is in the fields of medicine, biochemistry, and vaccines. In particular, it relates to vaccines that can be manufactured very rapidly and in huge quantities by using specialized viruses that grow in bacteria (rather than requiring bird eggs or other eukaryotic cells for viral incubation), and that can be administered by spraying a mist into the nasal cavities, without requiring needles or syringes.

At the most basic level, vaccines function by presenting foreign antigens, to cells that function as part of a mammalian immune system. This process allows an immune system to lay the groundwork for accelerated formation of antibodies that will help block and kill invading pathogens, if a need ever arises in the future. Beyond that basic level, the immune system is quite complex, and uses multi-step sequences involving numerous different types of cells. Chapter-length descriptions that provide good overviews are available in books such as Alberts et al, Molecular Biology of the Cell, or Guyton and Hall, Textbook of Medical Physiology. More detailed analyses are available in books such as Kuby Immunology, 6th edition (Kindt et al, W.H. Freeman, 2006), Immunology, 5th Edition (Goldsby et al, W.H. Freeman, 2002), and Immunobiology, 6th edition (Janeway, Garland Science, 2004). In addition, numerous review articles (which can be located through the U.S. National Library of Medicine database, at www.ncbi.nlm.nih.gov/entrez/query.fcgi) provide more information on specific aspects of vaccines or immunology. As examples, Thomas et al 2005 reviews genetically-engineered vaccines, while articles such as Gaubin et al 2003, Clark et al 2004, Wang et al 2004, and Miedzybrodzki et al 2005 focus on bacteriophages as vaccine carriers or delivery vehicles. Various materials also are available via the Internet. For example, the materials for numerous college or medical school courses on immunology are available (one example is at http://uhaweb.hartford.edu/BUGL/immune.htm). Glossaries also are available, such as at http://users.path.ox.ac.uk/˜scobbold/tig/gloss.html. Accordingly, background information herein on the immune system is intended only as an introduction and overview, and more detailed information is available from other sources.

Assuming that a reader understands the basic cell types and processes that are involved in immune responses, attention must be focused on vaccines that can be presented via nasal sprays, or other methods that involve contact with “mucosal” surfaces, discussed below. This route of administration has several potential advantages, compared to vaccines that require injection using needles (injection using needles is often referred to as “parenteral” administration, and usually often involves intramuscular injection). However, under the prior art, nasal and other mucosal vaccines have not reached a point where they are as effective as injected vaccines, and they have not been highly successful. The obstacles that must be overcome by nasal vaccines, and the progress they have made in recent years, are discussed in articles such as Eriksson et al 2002, Kiyono et al 2004, and Mestecky et al 2005. A mucosal vaccine that has been approved for use protects against influenza, and is sold under the trademark FLUMIST™ by MedImmune Vaccines, Inc., as described in articles such as McCarthy et al 2004, and on websites such as www.flumist.com, www.fda.gov/cber/flu/flumistqa.htm, and www.rxlist.com/cgi/generic3/flumist.htm. Failed efforts to develop mucosal vaccines are also known, and include, for example, Oravax Inc., which effectively collapsed in 1998 and was acquired by Peptide Therapeutics Group PLC, which later was taken over by Acambis PLC (www.acambis.com), which apparently is no longer attempting to develop any mucosal vaccines.

In the field of nasal vaccines, mucoadherents and adjuvants require attention. Mucoadherents (also called “absorption enhancers” or similar terms) will cause a nasal spray or similar formulation to either: (i) cling to the nasal sinuses (or other mucous membranes) for a longer period of time, or (ii) penetrate through a mucous membrane more rapidly, and in higher quantities. Either of those effects can increase the likelihood that an antigen in a vaccine will be noticed and recognized as foreign, by the immune system, in ways that will provoke a desired immune response. Accordingly, mucoadherents can be used with nasal vaccines as disclosed herein. Examples include chitosan and certain types of cyclodextrins, phospholipids, and other “bioadhesive” compounds, described in articles such as Davis et al 2003 and Zuercher 2003, and powders that convert into gels when they become wet, such as a compound from aloe vera plants sold by DelSite Biotechnologies under the trademarks GELVAC and GELSITE (www.delsite.com). More information on vaccines formulated as aerosols, powders, or similar forms is in articles such as LiCalsi et al 1999 and Chan et al 2006. It should also be noted that the nasal membranes are negatively charged; therefore, use of “cationisation” (described below) to impart a positive charge to vaccine particles may also be able to increase the contact time between the vaccine particles and their targeted membrane surfaces.

The subject of adjuvants is more complex, and requires careful attention.

### ADJUVANT DEFINITIONS, TYPES, AND EFFECTS

In the field of immunology, definitions of “adjuvant” have changed and evolved over time, and early definitions (which began to appear in the 1920's) may not encompass recent developments or definitions. Two functional definitions need to be considered; they say essentially the same thing, but with subtle differences.

In a first widely-used definition, an adjuvant is an agent or substance which, while not having any specific antigenic effect in itself, can stimulate an immune system in a manner that increases a response to a vaccine. In a second definition, an adjuvant is an agent or substance which, when combined with an antigen, reduces the amount of antigen that is needed in order to stimulate an effective immune response. A subtle distinction between those definitions is described below, since it is relevant to this invention.

In prior decades, and in most relevant articles and books, most adjuvants were separate compounds that were mixed with an antigen-carrying vaccine, to create an enhanced formulation. If an antigen in a vaccine formulation is accompanied by an adjuvant that functions, in effect, as an irritant that will provoke inflammation, swelling, or similar responses, the immune system will be alerted more rapidly to the type of challenge that arises when something foreign enters the body at a certain location. When an immune system has been “alerted” by an adjuvant that provokes or increases localized inflammation, it will use signaling and response mechanisms to send large numbers of immune cells to that site, to help fight the invasion. If numerous immune cells are sent to the site where a vaccine was administered, those immune cells will encounter and detect the antigen more rapidly, and in larger numbers. This will lead to a larger, faster, more effective response to the antigenic molecule, which is also present in the vaccine mixture, and which is injected into the same site as the adjuvant (this also explains why nearly all injectable vaccines are injected into muscle or skin tissue, rather than intravenously). Accordingly, an adjuvant can increase both the speed and the magnitude of an immune response to a vaccine.

Similar effects explain how adjuvants can help immune systems mount a more effective and consistent responses to a “weak” antigen. In this context, “weak” antigens are classified based on their effects in lab animals; weak antigens are those that are not strong enough to trigger full-scale antibody-producing immune responses, in the majority (or in a sizable fraction) of inoculated animals, unless an adjuvant is also administered. In this field, terms such as “weak” versus “strong” will depend on the animals used, the dosages used, and similar factors.

For decades, immunologists used a mixture called Freund's complete adjuvant (FCA), when they injected animals with a foreign antigen to provoke an immune response. The “complete” mixture contained pathogenic microbes called mycobacteria, which had been killed by heat or chemicals. However, FCA tends to provoke painful inflammatory responses, as part of its mode of action. Therefore, it is not used in human medicine, and when laws were passed in most countries (mostly in the 1980's) requiring that any pain and suffering imposed on lab animals must be minimized, other adjuvants began to be actively developed, as described in Eriksson et al 2002 (which focuses on mucosal adjuvants) and Guy et al 2005. As examples, Yuki et al 2003 and Lycke 2004 describe adjuvants in which a cholera toxin protein fragment is combined with an E. coli toxin fragment or a Staphylococcus aureus toxin fragment.

Cholera toxin (CT) and heat-labile E. coli toxin (HT) have been studied extensively, as potential mucosal adjuvants, because of several natural activities and effects they have. Since both toxins are polypeptides, the hope is that limited amino acid sequences from either or both of the CT and HT toxins can be isolated, and then incorporated into mucosal vaccines, to give such vaccines an enhanced ability to reach submucosal tissues containing large numbers of macrophages or other immune cells. Accordingly, animal tests and human clinical trials were commenced during the 1990's, using CT and HT as nasal adjuvants. However, the human trials had to be terminated, when animal tests indicated that vaccines carrying CT or HT sequences could travel through certain types of nerve fibers and enter the brain, causing brain inflammation. Those problems are described in a report from a July 2001 conference, entitled, “Safety Evaluation of Toxin Adjuvants Delivered Intranasally”, issued by the U.S. National Institute of Allergy and Infectious Diseases (NIAID), available at http://www.niaid.nih.gov/dmid/enteric/intranasal.htm. That report contains an extensive description of concerns and requirements that need to be addressed before any human clinical trials can begin again, using either CT or HT-derived toxins.

Rather than overcoming those problems, subsequent research identified even more problems that occurred when CT and HT toxin fragments when used as adjuvants for nasal vaccines. For example, Yoshino et al 2004 and van Ginkel et al 2005 reported that in animal tests, CT and HT toxin adjuvants created the following problems and warning signals: (i) they altered the normal patterns of antigen travel and immune cell responses (collectively referred to as “antigen trafficking”), which raises questions about whether an immune response to a vaccine would properly prepare a host to withstand an actual pathogenic infection; and, (ii) they provoked inflammation in the nasal tract.

Accordingly, the Inventor herein took a completely different route, and avoided any work with known toxins or toxin fragments as candidate adjuvants.

Genetically-engineered adjuvants offer the promise of incorporating a polypeptide sequence having adjuvant activity, into a “disarmed” (nonpathogenic) viral particle or fragment (or into an engineered chimeric protein) that also carries an antigenic polypeptide sequence. Therefore, in modern vaccines, adjuvants can be agents (such as known polypeptide sequences that have adjuvant-like activity) that are incorporated into the same particle or protein that also carries an antigen (such as a polypeptide sequence derived from a surface protein of a pathogen).

Returning to the two compatible definitions of “adjuvant” listed above, it recently has become clear that the formulation of vaccines as “microparticles”, having sizes that emulate various types of pathogenic microbes, can help trigger an effective immune response to a vaccine. That is not surprising, since macrophages and other immune cells evolved in ways that are ideally suited to combat such microbes. Therefore, by giving vaccine particles a certain physical size (measured in micrometers, or in some cases nanometers), the efficacy and speed of an immune response to an antigen can be increased, when the antigen is carried by a vaccine having an optimized particulate size. Accordingly, if the sizes of vaccine particles have been optimized, then the particles themselves (with optimal dimensions) can be regarded as having adjuvant activity and efficacy.

This concept embodies an important advance in the current invention, since the sizes and dimensions of filamentous bacteriophages, which can be used in this invention, appear to be ideal for triggering active and useful immune responses in mammals.

Finally, it also should be noted that adjuvants can be divided into two classes, based on their mechanism of action. “Delivery” adjuvants increase immune responses by means that increase the amount of contact between antigens, and “antigen-presenting cells” (APC's), using mechanisms such as described above. By contrast, “immunostimulatory” adjuvants activate the immune system by stimulating certain types of cells to release cytokines (i.e., hormone-type messenger molecules that trigger various types of cellular responses), or by other similar actions as described in articles such as Hunter (2002).

### Nasal and Other Mucosal Vaccines

One field that requires particular attention involves mucosal vaccines. This includes vaccines that are administered topically to a “mucous membrane”, also called an “epithelial” membrane. Such membranes include the mouth, nasal sinuses, vagina, rectum, etc.

The type of skin that covers most of the body and the limbs is made of epidermal cells. These cells are created by a “budding” process, in which an underlying layer of cells continuously creates a set of “partial” offspring cells. These cells become dehydrated and flat (“squamous”) as they move closer to the outer skin surface, and by the time they reach a subsurface layer called the “stratum corneum”, they are “enucleated” (i.e, they no longer contain a nucleus or chromosomes). By the time they reach the outer epidermal surface, they are effectively dehydrated and nonviable, and cannot support viral replication. This makes them ideal as an outer protective layer. They typically last for only a few days on the surface, then they peel and flake off, in the form of dried single cells or tiny clusters that usually are too small to be seen by the naked eye.

In contrast to dry epidermal skin, mucous membranes are covered by epithelial cells. Instead of providing a protective coating of flattened and dehydrated dead or dying cells, epithelial cells remain hydrated, to sustain their viability. After they reach an outer surface of a mucous membrane, they have a relatively short lifespan (typically about 4 to about 8 days), and during that period, they are much more active than epidermal cells, and generally are capable of supporting viral infections, especially if any lesions, abrasions, or other breaches in a mucous membrane enable viruses to penetrate through the outermost layers.

Accordingly, there is a high level of interest in mucosal vaccines. Among other advantages, mucosal vaccines can avoid any need for needles or syringes, which pose various problems (including hazardous waste disposal, theft by drug users, accidents that can infect healthcare workers, etc.), and which offend or intrude on the cultural norms of some societies. In addition, mucosal vaccines that are incorporated into genetically engineered plants offer the potential for helping reduce animal-related and food-related diseases.

Because of their potential, much effort and attention has been devoted to mucosal vaccines, as described in, e.g., Vajdy et al 2004, O'Hagan et al 2004, and Holmgren et al 2005. Reviews that focus on animal usage include Meeusen et al 2004; reviews that focus on efforts to develop mucosal HIV/AIDS vaccines include Belyakov et al 2004 and Stevceva et al 2004.

### Phages and Phage Vaccines

Since this invention involves a class of microbes called “phages”, as both active agents and adjuvants, background information needs to be provided on phages, and on prior efforts to use phages as vaccines.

The term phage is a shortened form of bacteriophage, derived from the Greek words for “eats bacteria”. Most phages will kill and destroy their bacterial hosts. However, a special class of viruses was discovered that can infect bacteria and then emerge, in large numbers, without killing the host cells. These specialized viruses are long and thin “filaments” which, after reproducing in a cell, are thin enough to emerge through pores or other outlets in a cell's membranes, without damaging the cell. Even though these types of filamentous viruses do not “eat” or kill their hosts, they were nevertheless called bacteriophages, or simply phages. Since they do not kill their hosts, they can be grown in bacterial cell culture at enormously high rates, so long as the bacteria are cultured with shaking or stirring, to prevent the phages from smothering the bacteria by sheer volume and bulk.

Several types of phages have been manipulated in ways that render them highly useful for genetic engineering. The most popular phages, used in laboratories around the world, usually combine all of the following traits:

(i) the DNA they carry is small enough to be easily manipulated;

(ii) they allow small or moderate-sized foreign polypeptide sequences to be inserted into segments of a first “coat” protein that is present in more than a thousand copies in each phage particle, without disrupting the ability of the modified phages to replicate in bacteria;

(iii) they can allow even larger foreign polypeptide sequences to be inserted into a second type of protein that is positioned near one end of the phage filament;

(iv) their DNA has been modified in ways that allow the phages to function both: (1) as bacterial plasmids, in double-stranded DNA form, or (2) as phage DNA, in single-stranded DNA form. This provides various advantages; for example, it allows antibiotic-resistance genes to be incorporated into the phages, in ways that allow host bacteria which have been infected by phages to be easily selected, from among large populations of bacteria, by culturing the bacteria in media containing the corresponding antibiotics;

(v) finally, these types of phages cannot infect mammals. They are totally nonpathogenic in animals and humans, and they can be used without the expensive and cumbersome precautions that are required when pathogenic microbes are involved.

Based on these and other factors, phage preparations have been developed in two different forms. Both forms must be understood, and kept distinct.

One form is a preparation containing numerous copies of single specific phage carrying a single foreign gene sequence (or “insert”). This type of preparation, often called a clonal or monoclonal phage, can be used for various purposes, such as for making vaccines. As an example, clonal phages containing a protein sequence from the human beta-amyloid protein (which forms beta-amyloid plaques, in the brains of people suffering from Alzheimer's disease) have been created by Beka Solomon, Dan Frenkel, and their coworkers at Tel Aviv University. These are described in a number of issued patents (including U.S. Pat. No. 6,919,075, by Solomon et al 2005, and U.S. Pat. No. 6,703,015, by Solomon et al 2004), published patent applications (including US 2005-0152878 and 2005-0053575, both by Solomon et al 2005), and articles (e.g., Frenkel et al 2002 and 2004). The hope and goal is that such phages, if used as vaccines, may be able to stimulate the production of antibodies or other immune responses that may be able to disassemble and dissolve beta-amyloid plaques, in the brains of Alzheimer patients. That is a noble goal, and some day, it may succeed. However, a clinical trial on a beta-amyloid vaccine (designated as AN-1792, by Elan Pharmaceuticals) had to be halted in January 2002, because patients who received the vaccine began to suffer from cerebral inflammation (an analysis is available in Schenk et al 2004). The cerebral inflammation may have been due to either or both of two factors: (1) an autoimmune disorder may have arisen, when the immune systems of those patients began attacking a protein that occurs naturally in the blood and brain; and/or (2) beta-amyloid plaques may function as a type of “plumber's putty”, which controls and reduces the leakage of blood out of capillary walls that have become thin and fragile, in the brains of elderly people; accordingly, if the sticky deposits that control that type of leakage are dissolved, blood leakage into the brain tissue may increase, leading to edema, inflammation, and other problems.

In addition to clonal (or monoclonal) phage preparations that can be used as vaccines or research, a different type of phage preparation that needs attention is called a phage display library (also called a phage library). This type of preparation is a mixture containing millions or billions of phages that carry different foreign polypeptide sequences. These phage libraries enable “screening” tests that allow certain specific phages to be selected and isolated, using one or more cellular or physiological processes. The phages that respond in a certain way, in a screening test, can be isolated, reproduced, and analyzed, to determine the foreign DNA and/or polypeptide sequence that caused those particular phages to behave in a certain way, in that particular screening test.

One important type of phage display library, which has been extensively developed by a company called Cambridge Antibody Technology (www.cambridgeantibody.com), contains billions of differing human gene sequences, obtained from human populations that included a wide variety of races and ancestries. The foreign gene and protein sequences, in the Cambridge Antibody phage display library, were derived from the “single-chain variable fraction” (scFv) portions of human antibodies. Because of how the foreign gene inserts were isolated, this phage library is highly useful in immunological research, and it was used in certain screening tests described below. This type of phage library is described in more detail in articles such as Pini et al 2000, and on the Cambridge Antibody Technology website.

Other known types of phage display libraries contain foreign DNA sequences with either random or controlled nucleotide sequences, which will be expressed into random or controlled variations in the coat proteins of phages.

By using cell culture tests to screen large numbers of phages from either type of phage library, the particular phages which happen to carry foreign DNA and peptide sequences that trigger certain types of cellular or physiological reactions can be isolated. Subsequently, after a “best performing phage” has been identified by an early round of screening, its foreign gene sequence can be used as the starting point for additional rounds of mutation and screening, using either random or controlled mutagenesis.

Due to various factors (including very fast replication, optimal particle sizes, and certain protein “display” traits, which includes the fact that inserted polypeptide sequences will be exposed and accessible on the outsides of the phage particles, rather than hidden inside the phages), various authors and researchers have recognized and reported that bacteriophages may someday be useful as vaccines. Articles that propose such use include Miedzybrodzki et al 2005 (“using natural agents such as bacteriophages as a weapon against pathogenic viruses could be an attractive and cost-efficient alternative, and further studies are urgently needed to test this possibility”), Koch et al 2004 (subtitled, “alternative and experimental approaches”), and Clark et al 2004 (“work has shown that whole phage particles can be used to deliver vaccines in the form of immunogenic peptides attached to modified phage coat proteins or as delivery vehicles for DNA vaccines, by incorporating a eukaryotic promoter-driven vaccine gene within their genome. While both approaches are promising by themselves, in the future there is also the exciting possibility of creating a hybrid phage combining both components to create phage that are cheap, easy and rapid to produce and that deliver both protein and DNA vaccines via the oral route in the same construct”).

However, despite their apparent potential, there has been very little commercial or industrial interest in using phages as actual vaccines, in human medicine. The reasons for the apparent lack of commercial and industrial interest are numerous, and include: (i) the unwillingness of large pharmaceutical companies (which presumably are the only companies that could afford a major research project that might be able to create a completely new and different class of vaccines) to risk hundreds of millions or even billions of dollars, on a project that may never succeed; (ii) the reluctance of large and well funded companies to even try to begin all of the necessary testing that presumably would be needed, to prove very high levels of safety, for vaccines intended for human use; (iii) the presence of enough speculative and suggestive proposals concerning possible phage vaccines, in the research literature of the 1980's and 1990's, to clutter and confuse an assessment of the prior art, making it difficult to reliably predict whether any strong patent protection could be gained if a company did invest large amounts of money and managed to succeed, by means of an approach that arguably used various suggested methods and components; (iv) the reluctance of large pharmaceutical companies to try to develop new and different products that might end up cannibalizing the sales and profits they already enjoy from their existing vaccines or pharmaceuticals; and, (v) the additional investment and profit risks that would arise if a highly effective vaccine can be manufactured rapidly, in huge quantities and at very low cost, in numerous countries where patent rights or other intellectual property protections cannot be enforced in a practical and economic manner.

Despite those problems, researchers in a position to do so have an obligation to try their best, to develop better vaccines. Despite various improvements, most vaccine production today uses technology that is decades old, and the old technology has failed, quite seriously, to accomplish several hugely important goals. As hugely important examples, no adequate vaccines have been created for AIDS, malaria, tuberculosis, or several other major diseases. As another example, there appears to be a general consensus among scientists and governments that a recently-emergent form of “bird flu” will someday mutate into a form that will be transmitted from humans to humans, and when that happens, the stage will be set for a worldwide pandemic that likely will kill many millions of people. However, a strong presumption arises that old manufacturing technologies, which mainly use bird eggs as incubators, is not likely to work well, for making vaccines against a virus that aggressively kills birds.

Accordingly, attention needs to turn to a different set of recent advances and discoveries, from a completely different line of medical research, which may allow certain methods from a different realm of art to be imported into the field of vaccine development, manufacturing, and use. That other field of research is briefly summarized below.

Delivering Polypeptides Through the Blood-Brain Barrier, Using Ligands that Enable Neuronal Endocytosis and Transport

The inventor herein, Dr. Ian Ferguson, is the first-named inventor of a separate patent application, published in April 2003 as Patent Cooperation Treaty (PCT) application WO 2003/091387. Its title is, “Non-Invasive Delivery of Polypeptides Through the Blood-Brain Barrier, and In Vivo Selection of Endocytotic Ligands”. The contents of that application are incorporated by reference, as though fully set forth herein.

That invention can be regarded as comprising two main components, which interact with each other to provide a complete operative embodiment. The first portion can be briefly summarized as follows:

1. Using specialized gene vectors (such as certain types of disarmed viruses that carry inserted “passenger” genes), it is possible to transfect certain types of neurons that “straddle” the blood-brain barrier (BBB). Such BBB-straddling neurons include, for example, olfactory receptor neurons (which have tips that are accessible in the nasal sinuses), and various neurons with tips that can be reached by liquids injected into the tongue, or into various muscles.

2. If the gene vectors have certain types of properly-selected proteins on their surfaces (proper selection involves the second part of the invention, described below), they will be taken into the accessible tips of the BBB-straddling neurons. This type of transport, into the tips of neurons, uses “endocytotic” receptors on the exposed and accessible tips of the neurons. Endocytosis is a well-known process, and a number of endocytotic receptors that appear on the surfaces of certain types of BBB-straddling neurons have been identified and fully sequenced.

3. After such genetic vectors have been taken into the accessible tips of the neuronal fibers, they can be transported from the neuronal tip, to the main cell body, by a natural process called “retrograde transport”.

4. The trick to activating and driving both: (i) entry into the neuronal tips, using endocytotic receptors, and (ii) internal transport to reach the main cell bodies of the neurons, is to find and use an effective “transport” protein (which can also be called a delivery, carrier, or locomotive protein, or similar terms) that will activate and drive two different processes, which are endocytotic uptake into the neuron, and retrograde transport within the neuron. A method for screening and identifying such proteins, using a phage display library that provided billions of candidate protein sequences, and that used a screening test to select, identify, and isolate a few such proteins which actually function in the desired manner, is described below, in the discussion of the second major component of this invention.

5. After a genetic vector has entered a neuronal fiber and has been transported to the main cell body, the gene carried by the vector can be expressed into proteins, and the proteins can be provided with a “leader sequence”, which can enable two functions (i) transport of the protein molecules by neuronal fibers that travel deeper into the brain; and, (ii) secretion and release of the proteins, at the “innermost” synapses of the neuronal fibers.

This approach, using specialized genetic vectors described in more detail in PCT patent application WO 2003/091387, provides an effective non-invasive method for delivering diagnostic or therapeutic proteins into BBB-protected brain or spinal tissue. Although confirmatory data were not available when that patent application was filed, it has been confirmed that this method of delivery functions effectively in animals, and efforts are being made to move it closer to human clinical trials.

That comprises the first major component of the invention. As mentioned above, in order for it to work, the “transport” or “locomotive” proteins that are exposed on the surfaces of the BBB-penetrating genetic vectors must be able to activate and drive two different cellular processes, which are: (1) uptake into neuronal fibers, followed by (2) transport through the neuronal fibers, toward the main cell bodies of the neurons. To identify “transport protein” sequences that can accomplish both of those two objectives, the Inventor developed an in vivo screening process, which initially used the sciatic nerve bundle, and which later was extended to olfactory neurons.

In mammals, the sciatic nerve is the main nerve bundle that travels from the base of the spinal cord, through the hip and the leg, to the foot. To carry out an in vivo screening procedure which uses that long nerve bundle in rats, a “ligature” was created, by surgically placing and then tightening a loop made from a suture strand, around the sciatic nerve bundle near the knee of a rat. This created a form of stress which caused the affected neurons to “upregulate” (i.e., increase the expression and placement of) certain types of cell surface receptors, including the so-called “p75” receptor, which attempts to help nerve cells recover from injuries.

One week later, that ligature near the knee of the rat was removed, and the sciatic nerve bundle was completely severed at that location. The exposed end of the nerve bundle was packed inside a sleeve that held a gel material containing a phage display library, with millions of candidate phage particles carrying different foreign protein sequences in their coat proteins.

During that same operation, a different ligature loop was placed and tightened around the same sciatic nerve bundle in an “upstream” location, near the hip. That ligature was tightened, to block the retrograde travel of molecules that otherwise would naturally flow through the nerve fibers, toward the spinal cord.

A suitable period of time (about 18 hours) was allowed to pass, to enable phages that happened to be carrying efficient “locomotive” protein sequences (in the coat proteins of the phages) to be both: (i) taken inside the neurons, and (ii) transported in a retrograde direction, through the thigh, toward the spinal cord. After that period of time passed, the animal was sacrificed, and a short segment of the sciatic nerve bundle, immediately below the ligature loop near the hip, was harvested.

Since the phage harvesting site (near the hip) was more than a centimeter away from the placement site (near the knee), the only phages that could reach the harvesting site were phages that had been taken inside a neuronal cell fiber, and that had been transported toward the spinal cord. Since those phages, traveling inside the nerve bundle, could not cross the constriction created by the tightened loop of suture thread near the hip, the fluid that was attempting to flow through the nerve bundle, toward the spinal cord, caused the phages to cluster and crowd into the nerve region immediately “distal” to the ligature (i.e., on the “downstream” side of the ligature, distant from the spinal cord). This allowed the phages of interest to be harvested, by isolating and removing a short segment of the sciatic nerve bundle, immediately downstream from the ligature at the hip. The phages were removed from the neuronal fiber segments, by using a buffer and enzyme mixture to digest the cell membranes (which are made of lipids) but not the virus particles (which are covered by proteins). The phages were then replicated in bacterial cells, to provide an “amplified” population.

The resulting mixture of phages from one round of screening was then screened again, using the same process. The screening procedure can be repeated any number of times, using an “enriched” phage population from a prior screening cycle as the starting material for a subsequent screening cycle.

Out of the many millions of candidate phages that were present in a phage library that was screened in that manner, a few phages were selected and isolated. The selection process ensured that those particular phages contained foreign polypeptide sequences that activated and drove both: (i) endocytosis (i.e., uptake and entry) of the phages into the neuronal fibers; and, (ii) retrograde transport inside the neuronal fibers (i.e., transport from a neuronal fiber tip, toward the main cell body of the neuron).

The above-cited PCT application WO 2003/091387 stated that similar techniques could be used to screen phage display libraries, to select phages that: (1) would enter the tips of olfactory receptor neurons, which are accessible on the surfaces of the nasal sinuses (i.e., the neuronal tips are not protected by the blood-brain barrier); and, (2) would be transported through the neuronal fibers, into brain tissue that is protected by the BBB.

Those comments were supplemented and confirmed by additional work that was disclosed in PCT application PCT/IB2005/04077, published as WO 2006/070290, by the same inventor herein. Briefly, it was realized that the rates of uptake of phages into the tips of olfactory receptor neurons, and the transport of the phages into brain tissue by the neuronal fibers, could be used to diagnose, measure, and quantify the health and vitality of neurons and brain tissues in people suffering from neurodegenerative diseases, such as Alzheimer's disease. This arises from the fact that endocytotic uptake and axonal transport are important processes in olfactory or other adjacent neurons, and if a certain set of neurons is losing or has lost the ability to carry out those processes, those losses can reveal the health and condition of those neurons, and of the neuronal networks they participate in.

To demonstrate that approach in rats or mice, fluorescently-labeled phages were used. Fluorescent labels can be identified and tracked visually, when thin slices of tissue from a sacrificed animal are analyzed under ultraviolet or similar light. However, in human use, tissue sections will not be available, so other types of labels must be used, such as (for example) short-lived isotopes that will show up in non-invasive imaging methods, such as single positron emission computerized tomography (SPECT) scans, computerized axial tomography (CAT) scans, magnetic resonance imaging, etc.

The results of that research made it clear that a number of phages had indeed been selected and taken in by neuronal fiber tips in the nasal sinuses, and had been transported through the nasal sinuses, toward and into the brain.

During the course of that work, which involved olfactory receptor neurons, it was realized by the Inventor herein that an entirely different type of use might become available, which could lead to the development of vaccines rather than diagnosis and treatment of brain disorders, if certain changes were made in the approach.

Understanding those changes, and how nasally-administered vaccines as described herein can be made to work, requires an entirely different set of background art to be reviewed and analyzed. Therefore, attention must now turn to a specialized type of tissue (called NALT tissue) that belongs to the immune system, and to differences between the “innate” immune system (which does not generate antibodies), and the “adaptive” immune system (which generates antibodies).

### NALT Cells and Tissues

The acronym “NALT” refers to “nasopharyngeal-associated lymphoid tissue”. The terms “nasopharyngeal” and “lymphoid” both need to be understood.

“Naso-” refers to the nose, including the nasal sinuses. “Pharyngeal” refers to the pharynx, which is a transitional region in the back of the throat, below or behind the mouth, but above the esophagus. Therefore, “naso-pharyngeal tissue” includes tissues located in the nasal sinuses, in the rear portion of the mouth, and/or in the upper throat region.

“Lymph” refers to watery fluids and cells that permeate out of blood vessels and then travel through the soft tissues. Instead of containing cells packed closely or tightly together, soft tissues contain a substantial fraction of extracellular water (typically about ⅙, by volume), in a “gel” matrix held together by proteins. The extracellular fluid in tissue gel allows nutrition to reach cells that are not directly adjacent to a blood vessel, and it also allows certain types of immune cells to permeate and travel through soft tissue, as described in more detail below. Those immune cells slowly travel to lymph nodes, which are highly important in the immune system. Accordingly, the term “lymphoid” has two distinct but heavily connected and interrelated meanings One meaning refers to the watery fluids that slowly pass and permeate through soft tissues; the other meaning refers to specialized immune cells and tissues that use, handle, or travel in lymph fluids.

By combining “nasopharyngeal” with “lymphoid”, the phrase “nasopharyngeal-associated lymphoid tissue” (NALT) refers to and includes several specialized sets of tissues in the nose, mouth, and throat region, which form important components of a mammalian immune system. Because the last word is “tissue”, the acronym can be used as a noun; however, it is also commonly used as an adjective, so “NALT cells” is correct usage, and “NALT tissue”, although not truly proper, should be politely tolerated. “NALT” is sometimes referred to as nose or nasal tissue, but that definition might exclude the tonsils or adenoids, which are NALT cells, so it is not used herein.

Two similar acronyms that are often used are GALT (gut-associated lymphoid tissue, which includes specialized intestinal tissues called “Peyer's patches”) and MALT (mucosa-associated lymphoid tissue, which includes both GALT and NALT cells). Both NALT and GALT tissue structures involve a layer of epithelial membrane cells (often referred to as M cells; the “M” initially referred to “membrane”, since they have specialized structures that appear only on the outermost surfaces of the mucosal layers, but M cells are sometimes also referred to as mucosal cells and/or as microvilli or microfold cells). A layer of M cells rests on top of an underlying cluster or nodule of tissue called a “subepithelial lymphoid follicle”. The M cells contain finger-like and/or heavily folded protrusions, often called microvilli or microfolds, which increase their surface areas and their ability to contact and interact with molecules that are being inhaled, or that are passing through the digestive tract. They began to receive serious attention in the 1980's, and over a hundred review articles describe their structures, activities, and roles in the immune system, as well as various efforts to exploit and use them for vaccines or other research or medical purposes. Such reviews include Kuper et al 1992, Ermak et al 1998, Kozlowski et al 2002, Man et al 2004, Jepson et al 2004, Holmgren et al 2005, Brayden et al 2005, and des Rieux et al 2006.

As discussed herein, M cells are regarded as a component of NALT or GALT tissues, and as NALT or GALT cells. Because of certain embryological factors, which focus on the origins of cells rather than their functions, some authors may regard or refer to M cells as being a cooperating but distinct layer, which is not a part of the actual immune system. Such distinctions are merely semantic, so long as a reader understands that M cells provide a surface layer of cells that have active and crucially important sampling and transport roles, resting on top of an underlying layer, cluster, or follicle of immunoactive cells.

NALT cells and tissues are highly important to the immune and allergic systems, since many pathogens, allergens, and other compounds are inhaled, and their first contact in a mammalian body occurs in the nasal cavities. Therefore, mammals evolved with certain types of specialized cells that are exposed on the surfaces of the mucous membranes in the nasal sinuses and mouths, which are active components of the immune systems. When these cells encounter a protein and recognize it as foreign, they effectively “grab” the protein and help deliver it to a lymph node, so that other cells of the immune system can process it, and can create antibodies that will help the body defend against the foreign antigen, if appropriate.

Since the Inventor herein was familiar with NALT tissues, and since he was already working on methods for using phage particles to deliver therapeutic proteins (such as nerve growth factor) into brain tissues via olfactory receptor neurons that have accessible tips in the nasal sinuses, he realized that NALT tissues offered a potential for similar but different types of transport activities that can be triggered by foreign proteins, using immune cells that can travel in the fluid drainage system provided by lymph. That is a very different mechanism, compared to neurons having long fibers that will transport nerve growth factor or other therapeutic proteins into brain tissues, by means of endocytosis followed by retrograde flow within a neuronal axon.

Accordingly, to adequately explain this offshoot, which moved away from treating neurodegenerative diseases and which focused on immune systems and vaccine development, some background information needs to be provided on certain components and processes of the immune system. The following three subsections, all within the Background section, are an effort to provide that information. These are intended as introductory summaries only, and more detailed information is available in various reference works, including various books and review articles cited above.

Innate Vs. Adaptive Immune Responses

A complete mammalian immune system actually comprises two different subsystems, which are called the innate immune system, and the adaptive immune system. The innate system is “hard-wired” and works very rapidly, while the adaptive system takes much more time to generate a response. This is analogous to the way a nervous system enables both: (1) hard-wired and very rapid “reflex” responses, such as withdrawing a finger immediately, when a hot surface is touched; and, (2) learning, which takes longer, but which can accomplish much more complex and sophisticated tasks.

The adaptive system requires and uses antibodies, which require participation by several different types of white blood cells, including B cells, T cells, helper T cells, killer cells, etc. This response takes several days to complete, and any delay lasting that long would allow most microbes (which can reproduce many times faster than mammalian cells) to generate huge numbers of invading microbes, before a complete antibody response can move into action. That delay period explains why vaccines, if prepared and administered in ways that allow an animal's immune system to partially get ready, in advance, before an infection even begins, can make a huge difference in how severe a disease or infection will become.

That delay period (i.e., the fact that several days will pass, before an animal's adaptive immune system can respond fully) also explains why most multi-cellular organisms evolved with an additional set of specialized proteins and cell types that form an “innate” immune system. This “innate” system can respond almost immediately, to help the body fight and slow down a set of invading microbes, while reinforcements (which are analogous to heavy artillery, including antibodies, B cells, T cells, killer cells, etc.) are being prepared, produced, and moved into position. Accordingly, the innate immune system responds and acts rapidly, as a first line of defense, without having to wait for days; then, while the innate immune system is slowing down the invaders, an adaptive (antibody) response is planned, organized, and made ready.

The innate immune system also is regarded and sometimes referred to as a “primitive” immune system. Since most invertebrate animals do not have full immune systems with antibodies, their only line of defense against microbes is their innate immune system.

As life on earth evolved into larger and more complex forms, innate (primitive) immune systems were already up and running, by the time adaptive immune systems began to evolve. Therefore, adaptive immune systems evolved in ways that use the innate immune system as a “springboard”. This was accomplished, at least in part, through the use and evolution of “pattern recognition receptors” (PRRs), which evolved in vertebrate animals as a response to “pathogen-associated molecular patterns” (PAMPs) in pathogenic microbes.

Most of the important groups of pathogenic microbes share certain types of “highly conserved” amino acid sequences, in certain domains of certain proteins. These highly-conserved domains of important proteins are driven by basic biological and biochemical needs and constraints. This prevents microbes from being able to rapidly mutate away from the patterns that work well for them, since deviations away from crucial and well-designed systems are much more likely to be detrimental, than favorable. Therefore, PAMPs are embodied by general patterns, rather than specific structures.

For example, animal cells do not have hardened cell walls; instead, they are enclosed within flexible outer membranes. By contrast, many types of microbes have cell walls, which allow those microbes to withstand fluctuations in their surroundings that would kill animal cells. Therefore, a number of molecules that are used to build microbial cell walls (such as certain types of lipoproteins, lipopolysaccharides, and peptidoglycans) came to be recognized as pathogen-associated molecular patterns (PAMPs) by the innate immune systems of small invertebrate animals (and, eventually, by vertebrate animals as well).

As a second example, animal cells do not have or use certain types of protein complexes that are highly conserved in “flagella”, which are the moving whip-like strands that E. coli and various other types of bacteria use to move about, in a liquid. Therefore, flagellar proteins from bacteria became another pathogen-associated molecular pattern.

As a third example, the genes of vertebrates evolved in ways that have two general traits: (i) methyl groups are gradually bonded to cytosine nucleotides, as an animal ages; and, (ii) there tend to be relatively few cytosine and guanidine nucleotides positioned immediately next to each other, in a DNA strand of a higher animal. Therefore, strands of DNA having unusually high numbers of unmethylated cytosine and guanidine nucleotides, adjacent to each other, in the body of an animal larger than a rodent, indicates that a microbial invasion probably is occurring. Therefore, DNA strands having large numbers of unmethylated cytosine-guanine dinucleotide pairs (this pattern is referred to as a “CpG motif”) can activate an important class of “toll receptors”, discussed below.

The patterns listed above are just a few examples of pathogen-associated molecular patterns (PAMPs). These pathogenic microbial patterns led to the evolution of corresponding “pattern recognition receptors” (PRRs), initially in invertebrate animals, as part of their innate immune systems. These “receptors” are not limited to the standard types of cell-surface receptors found in mammals; instead, they also include other types of molecules that can effectively latch onto (or otherwise respond to) one or more pathogen-associated molecular patterns that are present in various important classes of pathogenic microbes. This leads to a discussion of “toll receptors”.

### Toll Receptors

An important subclass of “pattern recognition receptors” in animals is called “toll receptors”. These were first seen in Drosophila (i.e., small fruit flies that are widely used in genetic research). They were called “toll” receptors not because of any particular function, but because “toll” is a German word for “amazing”. Since their definitions and boundaries are not always clear, especially as the DNA and amino acid sequences for toll receptors in mice are used to search for “homologous” sequences in humans, they are often referred to as “toll-like receptors”, using the acronym “TLR” followed by a number, such as TLR4 or TLR9.

As this is being written, in early 2007, at least 11 types of toll-like receptors have been identified in humans; in addition, two other types are believed to exist in mice, and there is heavy overlap between human and mice TLR's. Each toll-like receptor type is associated with at least one type of microbial “ligand” that will activate that subclass of TLRs. The known TLR types, and the ligands which activate them, are listed and described in literature that can be downloaded at no charge from Imgenex (www.imgenex.com), a company that sells ligands, antibodies, and other compounds used in research on toll receptors. Illustrated summary pages are available at http://imgenex.com/view_data_page.php?id=27 and http://imgenex.com/Toll-likeReceptors.php, and a 34-page brochure entitled “Toll-like Receptor Research Tools” can be downloaded via http://imgenex.com/dfiles/DownloadN-32.pdf. Review articles that describe toll receptors and their ligands include, for example, Hemmi et al 2005, Alexopoulou et al 2005, and Pasare et al 2005.

Not all TLRs function in the same way, and they can be grouped into two main classes, in terms of location. In one class of toll receptors, which includes TLR1, TLR2, TLR4, TLR5, TLR6, and TLR10, the receptors straddle the outer membrane of an animal cell, with an extra-cellular portion, and an intra-cellular portion. If the extra-cellular portion binds to a microbial pathogen (or to an artificially-administered PAMP ligand), changes are triggered in the intra-cellular domain.

In the other class of toll receptors, which includes TLR3, TLR7, TLR8, and TLR9, the receptors are associated with membrane-enclosed organelles (also called vesicles) located inside a cell. TLR9 receptors are of particular interest herein, since they are located inside macrophage cells (described below), and since they have been studied extensively. TLR9 ligands are fairly well understood, and are described in articles such as Krieg 2002 and Klinman et al 2004.

Regardless of which subclass of toll receptor is involved, activation of a toll receptor will trigger a series of reactions that will lead to movement (translocation) of certain types of DNA transcription factors. These initial responses lead to genes being expressed that will generate and/or trigger the activation or release of certain cytokines, which will then trigger a cellular or multicellular reaction, such as an inflammatory response, or cellular release of various antimicrobial agents.

In summary, toll receptors recently have been recognized as important “gate-keepers”, with functions that can be regarded as comparable to sensors. They can play major roles in determining whether: (i) a complete immune response will be launched, in response to an apparent invasion by a pathogenic microbe; or, (ii) only a localized and/or allergic or tolerance reaction will be commenced.

However, because of their complexity, and in view of the number of different toll receptors that are known to exist, they cannot be triggered randomly or indiscriminately, without posing major risks of provoking autoimmune or other potentially serious disorders. Therefore, what is needed (and what is provided by the invention herein) is a process, and a set of triggering or activating reagents, which can selectively activate only certain targeted types of toll receptors. Those processes and reagents are described below.

It should also be noted that various adjuvants used in the prior art (as accompanying agents that can help trigger a full-blown immune response, when coinjected along with a particular antigen) are actually compounds that trigger and activate toll receptors. As examples, proteins from bacterial flagella, and polypeptide segments derived from a heat-labile endotoxin protein from E. coli, have been used for decades as adjuvants, in vaccine formulations. After the role of toll receptors was recognized, it became clear that those adjuvants were activating certain classes of toll receptors, in ways that helped activate full-scale immune responses.

As described in more detail in the Detailed Description section below, one of the most important aspects of this invention is that phage vectors as disclosed herein can be modified so that they will specifically target certain types of toll receptors that are located entirely inside immune cells, such as TLR9 receptors. This can be very advantageous, since it reduces and minimizes the risks of triggering system-wide shock, a “cytokine storm”, or other undesired responses that would be more likely to occur if toll receptors on the external surfaces of macrophages were being targeted. By using a screening process that will select for “locomotive” proteins that can carry out a complex two-step process (i.e., entry into a macrophage cell, followed by activation of TLR9 or other toll receptors located inside those cells), a higher level of safe and selective targeting can be provided. This is discussed in more detail below.

### Types of Immune Responses and Cells; Monocytes and Macrophages

Different types of immune reactions can be created in response to various types of triggering agents. As one example, a vaccine might generate either an undesired allergic reaction, or a desired antibody-forming response. As another example, while the vast majority of vaccines (including essentially all vaccines used in preventive inoculations) are designed to provoke antibody formation, some vaccines used to treat specific patients suffering from cancer or other diseases are designed to provoke “cell-mediated immune responses”, which involved specialized cytokines (signaling molecules) and activated T cells, without involving B cells or creating antibodies.

Therefore, an important goal in vaccine development is to identify and use vaccine components and formulations that will maximize the likelihood of a desired response (such as antibody formation), while minimizing the risk of unwanted responses (such as allergic reactions).

Some of the cell types and components that determine which pathway an immune system will take, in response to foreign substances such as microbes, pollen, or vaccines, include:

(1) there are two different classes of “major histocompatibility complex” (MHC) receptors; these are known as MHC-1 receptors (present on nearly all cell types) and MHC-2 receptors (present only on certain types of immune cells);

(2) there also are two different classes of T cells, known as “cytotoxic T cells” (which will directly kill animal cells that harbor pathogenic microbes), and “helper T cells” (which secrete messenger molecules such as lymphokines, interleukins, or cytokines, which activate other cells in the immune system);

(3) helper T cells are further subdivided into two important classes, which are called TH1 cells (which secrete interleukin-2 and gamma-interferon), and TH2 cells (which secrete interleukin-4 and interleukin-5);

(4) there are five different classes of immunoglobulin molecules, which are: (1) IgM globulins, which are the initial forms of globulins sometimes bound to the cell membranes; (2) IgG molecules, which are the classic Y-shaped antibodies that are secreted by B cells to fight invading microbes; (3) IgA globulins, which are secreted by mucous membranes, and which latch onto airborne or other arriving pathogens in an effort to inactivate them and deliver them to the stomach or secrete them from the body; (4) IgD globulins, which are membrane-bound antibodies produced by B cells early in their life cycle; and, (5) IgE globulins, which are involved in allergic reactions.

In general, MHC1 and Th1 components work together to promote “cell-mediated” responses (also called cytotoxic responses), which involve cytokines and other signaling molecules and helper T cells, without requiring antibodies. By contrast, MHC2 and Th2 components work together to promote antibody formation. Those two types of responses are not mutually exclusive, and they can reinforce and supplement each other, to produce exceptionally strong and powerful immune responses. Accordingly, if a single vaccine preparation can provoke both type of responses, it may be able to provide better protection against various types of diseases. However, issues of timing may also be important, in such matters. The establishment of an “anamnestic” (non-forgetting) antibody-producing capability (which can be held quietly in reserve until needed, and then activated quickly) is highly beneficial, even if it occurs years before an infection. However, it is not always desirable to increase cytokine activities or helper T-cell functions, unless and until an actual infection is occurring. Such matters, which are understood by those skilled in the art, need to be taken into account in designing and administering different types of vaccines to healthy people, or to patients who are already sick.

Another possible option, for an immune system that detects a foreign antigenic substance, is to do nothing of significance, and not mount a substantial response. This type of nonaction, usually referred to “tolerance”, plays crucial roles in avoiding autoimmune diseases, allergic overreactions, and other problems. It is governed by complex regulation, rather than inattention. Holmgren et al 2005 offers a good overview of tolerance and how it may be manipulated in controlled ways to help treat autoimmune diseases, allergies, etc. Nevertheless, tolerance is an adverse result, when referring to vaccines, since it means that a vaccine failed to create a desired result.

Because the large majority of vaccines are designed to trigger antibody responses (rather than cell mediated MH1/Th1 responses involving cytokines and T cells), the discussion below focuses on antibody formation as a desired response, and on allergic reactions as an undesired response.

The fact that some vaccines can provoke either an antibody response or an allergic reaction arises largely from the fact that immune cells called “macrophages” must commit to triggering either a desired systemic response, or an undesired localized response, soon after the macrophages encounter and bind to what appears to be a foreign invader. As a brief overview of how this happens, white blood cells called “monocytes” (which were given that name because they have a single clear nucleus, unlike similar cells called “neutrophils” that have their chromosomes divided into several clusters) circulate in the blood, inside blood vessels. While in circulating blood, monocytes are relatively dense and compact. They have surface molecules that grip the interior walls of capillaries and then permeate through the capillary walls, causing monocytes to leave the circulating blood and enter the tissue itself. After they leave a blood vessel, the monocytes swell to a larger size. When that occurs, the enlarged cells become macrophages; as described below, they are also called phagocytes or phagocytic cells, because of how they surround and engulf small particles.

Macrophages travel slowly through soft tissues, within the lymph fluid. As mentioned above, soft tissues contain extracellular watery fluid (typically about ⅙ by volume) in a “gel” form that is held together by thick bundles of collagen, and by protein filaments coated with sugar groups, called proteoglycans. The slow travel of macrophages, through lymph fluid, is analogous to a policeman walking a beat rather than riding in a car; instead of being in a hurry to get somewhere, the policeman is mostly just looking around, to see if anything is present that should not be there.

Neutrophil cells should also be mentioned briefly. They act in a manner similar to monocytes, but important differences exist. Neutrophils normally remain in circulating blood until they receive a signal (such as from a cytokine, lymphokine, or other signaling molecule) indicating that an infection is occurring in a certain location. In response to such signals, neutrophils undergo a transformation, pass through a capillary wall, and enter the lymph fluid in the infected area of tissue. The neutrophils then swell to a larger size, and begin attacking the foreign particles. In some cases, they can engage in the classical form of phagocytosis (as described below), but more commonly, a neutrophil will trigger an “oxidative burst” when it senses that it has reached and contacted an invading pathogen. This “oxidative burst” creates and releases large number of “free radicals”, which are molecules having unstable and aggressively reactive oxygen atoms with unpaired electrons. Those radicals will attack and help destroy invading microbes, and they often kill the neutrophil as well. As a result, the whitish material called pus, in infected tissues, often consists largely of neutrophil cell remains.

That defensive process, by neutrophils, can be referred to as phagocytosis, but not all authors include or refer to it as actual phagocytosis, which focuses upon engulfing a foreign particle and taking it inside a defending cell. By way of analogy, if a man is using a knife and fork to cut apart a piece of meat, some people would refer to that preparatory action as eating, while others would say that eating does not actually begin until the person puts the food into his mouth. Those types of semantic distinctions are not important, so long as one understands the overall process.

When a true macrophage (rather than a neutrophil) encounters and recognizes a foreign cell or other particle (regardless of whether it is a virus, bacteria, pollen, etc.), it engulfs the foreign cell. This process is phagocytosis; as mentioned above under the definition of bacteriophages, “phago” is derived from the Greek word for “eat”. In some cases, a macrophage can engulf and swallow dozens of bacteria or viruses. Since viruses are particles rather than cells, the word “phagocytosis” has come to refer to cellular ingestion of any type of relatively large particle (including, for example, particles made of starch or plastic, which can be fluorescently labeled in ways that enable the rates of phagocytosis to be easily measured, using automated machines such as flow cytometers). Phagocytosis is distinct from a similar process called “pinocytosis”, which refers to the ingestion of very small particles and/or liquids. In general, phagocytosis is regarded as the cellular equivalent of eating, while pinocytosis is the cellular equivalent of drinking

The process of phagocytosis is aided by several factors. For example, most living mammalian cells (including macrophages) have negative electrical charges on their surfaces. Therefore, the presence of negative charges on cells helps prevent macrophages from attacking living cells of the host. However, that electrical charge dissipates after a cell dies, enabling macrophages to “eat” dead cells so that their building blocks can be reused. This aids the gradual turnover and replacement of cells, in ways that keep soft tissues (muscles, organs, etc.) flexible and viable.

Importantly, many pathogens have positive charges on their surfaces. This helps them rapidly grasp and infect a mammalian cell, and that type of rapid action is hugely important for pathogenic microbes. However, the same positive surface charges that help pathogenic microbes attach to and infect mammalian cells, also helps macrophages recognize and destroy the foreign invaders.

It should also be noted that in addition to macrophages and neutrophils, certain other cell types also can perform phagocytosis, including Schwann cells, certain types of glial cells, and various classes of dendritic cells. Indeed, in the process of apoptosis (which all soft tissues use, to continuously replace aging cells with new cells), there is evidence that after a cell has died, adjacent tissue cells begin participating in phagocytosis, which can accelerate their ability to begin taking in nutrients and building blocks from a dead neighboring cell that is being recycled.

Accordingly, although the description herein focuses on macrophages, terms such as phagocytosis, phagocytic, and phagocyte can also apply to other cell types.

In addition, terms such as “phagocyte” and “phagocytic” are also used inconsistently for another reason. Some people use those terms to refer to macrophages and other phagocytic cells at all times, while others use those terms only when cells are actively engaged in phagocytosis.

Similarly, the terms “professional” and “nonprofessional” phagocytic cells apparently are not always used consistently. Some people refer to all macrophages and neutrophils as “professional” phagocytes, while others limit any references to “professional” phagocytosis as involving the processes that lead to antigen presentation. Since phagocytosis involved in apoptosis (i.e., the replacement of aging cells in soft tissues) does not lead to antigen presentation, even though macrophages are involved, potential conflicts between those different uses of the same term should be noted.

As mentioned above, when a macrophage encounters a foreign object, it changes shape, by extending projections (which can also be called fingers, “pseudopods”, or other terms) out from the main cell body. These projections begin extending around a foreign particle, in a way that enables the projections to meet and merge on the far side of the particle, effectively engulfing the particle and taking it inside the macrophage. This type of shape-changing is similar in several respects to the motions of amoeba and certain other types of microbes, when they encounter and engulf smaller microbes. In its earliest forms, phagocytosis enabled single-cell microbes such as amoebae to obtain nutrition. Subsequently, it became a crucial part of the “innate” immune system of very small animals, and still later, it expanded into a major part of the “adaptive” immune systems of larger animals. This evolutionary process, and the detailed cellular steps involved in phagocytosis, are described in reviews such Jutras et al 2005 (entitled, “Phagocytosis: At the Crossroads of Innate and Adaptive Immunity”), and Brumell et al 2003.

When a particle-engulfing process begins, a macrophage is sometimes called a dendrite, or a dendritic cell. Those terms are derived from a Greek word for branching, or treelike. Accordingly, the three cell types that are commonly referred to, in the literature, as “professional” phagocytes are macrophages, neutrophils, and dendrites. The terms “dendrite” and “dendritic cells” are not preferred herein, since they are more commonly used in medicine to refer to branching structures that occur among neurons, which use fibers to establish connections with other neurons.

When carried out by macrophages, the phagocytic engulfing process is activated by cell-surface receptors, usually called “phagocytic receptors” or “phagosome receptors”. These receptors are important to this invention, and they are discussed in more detail below, since phage vaccine cassettes have been identified herein that have been selected for their ability to activate and drive the process of phagocytosis, by reacting with phagocytic receptors. A number of phagocytic receptor types are known, including various subclasses of lectin receptors (e.g., McGreal et al 2004), Fc receptors (e.g., Swanson et al 2004), and complement receptors (e.g., Ishibashi et al 1990). When used with phage display libraries, the methods disclosed herein offer powerful tools for identifying other phagocytic receptor types as well.

When a phagocytic receptor has been activated by a microbe, or by a phage vaccine particle as described herein, the cell commences a series of steps, in which the engulfing cell extends two or more finger-like projections, around the sides of the particle that has become bound to the phagocytic receptor. Other cellular “organelles” that have their own membranes (including endosomes, and endoplasmic reticulum) are recruited to assist, and they begin contributing all or parts of their own membranes, to the rapidly-growing membrane “pocket” that is being formed around the particle that is being engulfed by the cell. Once the newly-formed membrane pocket completely surrounds the particle, it detaches from the outer membrane of the cell, thereby creating a separate bubble-like component, often called a “vesicle”, with its own membrane, floating inside the cytoplasmic liquid inside the cell that has engulfed the particle. After that vesicle detaches from the cell's outer membrane, it is called a phagosome.

A phagosome will then merge and interact with another class of cellular vesicles, called lysosomes. After a phagosome merges with one or more lysosomes, the combined vesicle is still called a phagosome, or it can be called a phagolysosome. Lysosomes are the main digestive components in eukaryotic cells; their internal fluids are very acidic, and they contain strong digestive enzymes, usually called lysozymes, hydrolyzing enzymes, or hydrolases. Accordingly, lysosomes contribute acids and digestive enzymes to phagosomes, and the resulting mixture kills and digests the foreign particle.

When a phagosome contains an ingested microbe, then (in at least some cases) the digestive process will not completely digest the microbe (i.e., it will not break apart the microbe all the way to the level of single amino acids, nucleotides, and other building blocks, which would thereby become nutrients for the cell). Instead, macrophages partially digest invading microbes, in ways that lead to presentation of antigenic polypeptide fragments (from a microbe, or from a vaccine particle) on the “tips” of specialized types of cellular “fingers” created by the macrophage. Those fingers, carrying exposed partial protein fragments from a microbe or a vaccine particle (the foreign protein fragments are “mounted on” MHC-I or MHC-II molecules, as mentioned above), will be “presented” to other types of immune cells, including B cells and T cells. Those B and T cells will then carry out the next set of steps, in the process of creating antibodies that will bind to the protein fragments that have been “presented” by the activated macrophages.

Accordingly, efficient binding of vaccine particles to phagocytic receptors on the surfaces of macrophages, and efficient entry of the vaccine particles into functional phagosomes inside the activated macrophages, are crucial steps in the overall process that leads to “antigen presentation” by macrophage cells.

That is important because, as described below, this invention has enabled phage libraries to be screened in ways have identified certain particular phages (from among a huge number of candidate phages, in a phage display library) which happen to be carrying surface polypeptide sequences that can potently activate and drive not just one or two important steps, but an entire sequence and series of steps that will potently activate a desired type of immune response. The necessary transport and delivery steps (all of which preferably should be promoted and driven by a single phage “cassette” particle, which can be used to carry, transport, and deliver a selected antigenic polypeptide sequence that is encoded by a foreign DNA insert, inserted into the cassette) include each and all of the following:
(1) cellular uptake (or entry, intake, ingestion, endocytosis, or similar terms) of the phage vaccine “cassette” particles, into NALT tissues that are exposed and/or accessible inside the nasal, mouth, and throat membranes (this includes both an outer layer of M cells, and NALT cells positioned beneath that surface layer);
(2) transport (or delivery, presentation, or similar terms) of the phage vaccine “cassette” particles, to the surfaces of macrophages formed from monocytes;
(3) binding of the phage cassette particles to one or more phagocytic receptors on the surfaces of the macrophage cells;
(4) activation of the phagocytic receptors, in a manner that leads to efficient intake of the phage vaccine cassette particles into properly functioning phagosomes, inside the macrophage cells.

Each and all of those steps, in that entire sequence and series of steps, have indeed been completed and accomplished, by a single particular phage that was very effective and efficient in driving each and all of those steps, in mice. Therefore, the use of multi-step screening methods that are disclosed herein, and that have been shown to be fully capable of identifying specific phage particles (from a large phage display library) that will efficiently drive that entire sequence and series of steps, can now be carried out for any other type of mammal (including rodents, primates, humans, livestock, companion animals, etc.), and for other vertebrate species having immune systems that create and use antibodies (which includes poultry and other types of birds).

### Antigen-Presenting Cells (APCs)

For completeness, it should be noted that several different types of phagocytosis occur. One important class involves a type of “cellular eating” that plays a major role in a process called “apoptosis”. In that process, dying or dead cells are engulfed, broken apart, and digested by macrophages, leading to the release and recycling of their building blocks, which are used to form new cells. Apoptosis enables soft tissues to continually replace old cells with new cells.

That type of phagocytosis is unrelated to how the immune system responds to microbes or vaccines, and to antibody formation. Accordingly, to avoid confusing this invention (involving vaccines and immune responses) with apoptosis and cell replacement (which is not relevant herein), the term “macrophage” as used herein is limited to cells that can be converted, under proper triggering conditions, into “antigen-presenting cells” (abbreviated as APC, with the plural forms APCs or APC's). Monocytes (i.e., pre-macrophage cells that have not yet left the circulating blood), macrophages that can be converted into APCs, and dendrites or dendritic cells that have commenced the process of engulfing and swallowing a microbe or vaccine, all fall within the definition of APCs, if they are involved in the immune system. More information on antigen-presenting cells can be found in numerous reviews, such as Trombetta et al 2005.

Similarly, terms such as “phagocytosis” and “phagocytes”, as used herein, are limited to the types of “particle swallowing” processes that lead to antigen presentation, rather than to the killing and recycling of aging cells that occurs in apoptosis and tissue renewal.

### Different Potential Pathways for Macrophages

As mentioned above, a macrophage that has engulfed a foreign particle generally will respond to one or more signals that will lead it down one of several diverging pathways. Accordingly, a macrophage that has encountered a microbe or vaccine particle must effectively choose between several options.

One set of options involves a choice as to whether it will stay at the site and engulf more particles (for example, some macrophages reportedly have engulfed as many as 100 bacteria), or whether it will begin traveling rapidly toward a lymph node, to alert other cells in the immune system that a problem has been detected so that a full response can be prepared as rapidly as possible.

A second set of options involves whether the macrophage will commit to generating either: (1) an antibody reaction, also called a “humeral” response; or, (2) a cell-mediated immune response, which will involved specialized activated T cells without involving B cells or antibodies.

If a macrophage commits to helping form a systemic response (which can be either an IgG antibody response, or a cell-mediated response), it will begin moving rapidly toward a lymph node, as though responding to signals indicating that an emergency has arisen, and it must hurry, before more invaders can multiply and cause more problems. During this process, the macrophage begins converting (or “maturing”) into an “antigen-presenting cell” (APC). In this form, using phago-lysosomes as mentioned above, it will semi-digest the particle it has ingested, and it will transport a partially-digested protein fragment, mounted on either an MHC-I or MHC-II complex, to the tip of one or more of its extended projections. This presents the antigen to other cells of the immune system, including B cells, T cells, and “helper T cells”, which will begin performing the next steps in the systemic response. In addition, a macrophage that has been activated in this manner will secrete certain types of messenger molecules, such as interleukin-1. These molecules, also called “co-stimulatory” molecules, help activate other types of immune cells.

Accordingly, macrophages occupy and perform a gatekeeper role, at a crossroads where the adaptive immune system (in vertebrates) branches out from the innate immune system (which originally formed in primitive animals, and which is the only immune system most invertebrates have). Most invertebrates have cells that can effectively act as macrophages, which will engulf and eat foreign invading pathogens. However, invertebrates do not have any additional components (including antibodies, B cells, and T cells) that can provide more sophisticated adaptive immune responses.

Because of how they evolved, macrophages in vertebrate animals stand at a point where two different pathways split and went in different directions. When a macrophage encounters a foreign invader, it must either commit to a primitive-type localized response (mainly involving phagocytosis of foreign or damaged cells and debris), or it must commit to a more complex, sophisticated, and time-consuming response to recruit and train an entire team of cells that will defend the organism.

Therefore, when a vaccine is used to create an antibody response, steps should be taken to steer and guide macrophages toward that desired response, rather than toward an allergic reaction, or a passive “tolerance” response. That type of “steering” is what well-selected and properly targeted adjuvants can help accomplish, as discussed elsewhere in this Background section, and it also can be promoted by the methods and reagents of this invention.

Except as specifically noted, any remarks in this application about vaccines refer to the types of vaccines that will help an animal develop a strong IgG-type and/or IgA-type antibody (or “humeral”) and/or a cell-mediated (or “cytotoxic”) immune response (or a combination both types of responses). Most such vaccines are designed to help humans or animals resist infections by pathogens; however, a number of vaccines are being tested in the hope that they will be able to help patients fight non-infective diseases, such as cancer, Alzheimer's disease, etc. Those types of vaccines are discussed in more detail below, and more information on them is available in numerous other sources. Similarly, various types of vaccines are being tested and developed that involve peptide hormones and/or hormone receptors, for purposes such as birth control, stimulating growth, and fighting certain diseases that are aggravated (or “fueled”) by hormones. As will be recognized by those skilled in the art, the teachings herein can be adapted for use in any such vaccines that involve antibody-forming and/or cell-mediated (cytotoxic) immune responses.

Accordingly, in view of all Background information summarized above and known to those who specialize in immunology, one object of this invention is to disclose a new approach to developing and manufacturing vaccines, using nonpathogenic bacteriophages that contain foreign polypeptide sequences that have been screened and shown to actively trigger NALT uptake, association, or other processing.

Another object of this invention is to disclose a new approach to developing and manufacturing vaccines, using nonpathogenic bacteriophages containing genes and coat protein polypeptides that can promote delivery of the vaccines (and/or antigenic proteins carried by the vaccines) to antigen-presenting cells, and/or to phagosome components within antigen-presenting cells.

Another object of this invention is to disclose new types of vaccines (which can use bacteriophages, other types of viral particles such as glycosylated viruses that infect eukaryotic cells, or cellular microbes) that have been enhanced by incorporating into them a “targeting-and-delivery” polypeptide sequence that will potently trigger and drive (i) intake into NALT and/or GALT cells and tissues, followed by (ii) phagocytic intake and processing by macrophages or other antigen-presenting cells. In addition to containing enhanced transport polypeptide sequences, such vaccines also will carry at least one antigenic sequence that lead to antibody formation, to help an inoculated animal or patient resist an invading pathogen, fight cancer cells or other disorders, etc.

Another object of this invention is to disclose new types of vaccine “cassettes” that can be used to insert any desired antigenic polypeptide sequences into a highly efficient delivery system that already contains: (i) a NALT-targeting, APC-targeting, and/or phagosome-targeting peptide sequence which will help ensure delivery of completed “cassette” vaccines into specific targeted cells or sites of the immune system; and, (ii) a toll receptor ligand that will help such vaccines reliably provoke desired immune responses, rather than allergic, tolerance or localized reactions, such as by activating one or more toll-like receptors.

Another object of this invention is to disclose new types of particulate phage vaccines, with at least one a NALT-targeting, APC-targeting, and/or phagosome-targeting peptide sequence and at least one selected antigen sequence, that can provoke strong and rapid antibody and other immune responses of the type that can subsequently help a host animal resist a pathogenic infection.

Another object of this invention is to disclose new types of particulate phage vaccines, with at least one component (which may comprise, for example, a DNA strand that contains CpG motifs) that functions as a toll receptor ligand, to help provoke strong systemic immune responses in inoculated animals and patients.

Another object of this invention is to disclose new methods to identify and isolate, from phage display libraries, ligands that will potently activate phagosome receptors.

Another object of this invention is to disclose uses for NALT-targeting and/or phagosome-targeting phage particles, in targeting the delivery of diagnostic and/or therapeutic payloads (such as DNA gene expression plasmids, tracking and/or imaging reagents, etc.) into selected phagocytic cells.

Another object of this invention is to disclose new types of vaccines that have been modified in ways that enable the phage particles to provide potent adjuvant activity while carrying antigenic protein sequences and/or nucleotide segments that function as toll receptor ligands.

Another object of this invention is to disclose new types of vaccines that can be manufactured very rapidly, in large quantities, and at low cost, using bacteria (or other cells that can be grown in stirred cell culture) as the host cells for incubation.

These and other objects of the invention will become more apparent through the following summary, drawings, and description.

### SUMMARY OF THE INVENTION

Methods and reagents are disclosed for using bacteriophages (i.e., nonpathogenic viruses that infect bacteria such as E. coli) to manufacture mucosal vaccines, which can be administered without requiring needles, such as via nasal sprays. The coat proteins of phage vaccines for nasal usage must contain foreign polypeptide sequences that will cause the phage particles to bind to and activate nasopharyngeal-associated lymphoid tissue (NALT) cells. Such phages have been and can be identified and isolated from a phage display library containing billions of candidate phages, by means of an in vivo screening process in which a phage display library is administered nasally to a lab animal, which is later sacrificed so that tissue samples can be harvested and treated, to extract phages that were taken in and transported by NALT cells. Additional screening tests on such “enriched” NALT-targeted phage populations have been and can be screened by additional screening tests, to identify subsets of any such NALT-targeting phages that will also potently drive phagocytic intake and processing, by macrophages or other antigen-presenting cells (APCs).

After a polypeptide sequence that potently triggers and drives both NALT intake and APC intake has been identified by screening of a phage display library, the DNA sequence which encodes that polypeptide can be used to prepare a “cassette” vector, which can receive and hold any additional foreign gene sequence, inserted into one or more surface proteins (such as one or more coat proteins, if filamentous phages are used as the vaccine particles). In one embodiment, the completed vector will contain, in an exposed and accessible location, one or more antigen sequences that will provoke an antibody response that will help animals fight an invading pathogen, such as viruses or other pathogens. In other types of vaccines, selected antigens can help a patient's immune system attack and destroy cancer cells, beta-amyloid plaques in Alzheimer patients, or other cells or materials that cause or aggravate noninfective and/or nonmicrobial disorders.

The resulting vaccines will contain a combination of useful components and traits, including the following:

1. they will incorporate and use “targeting” polypeptides that have been screened and selected for high levels of binding to, and transport through, specialized epithelial cells (often referred to as “M” cells that provide the surfaces of “nasopharyngeal-associated lymphoid tissue” (NALT) or similar mucous membrane surfaces;

2. the NALT-targeting polypeptides that will enable and drive the first step in the desired transport sequence will also be selected and screened to ensure that they will stimulate active phagocytosis by macrophage cells (which also can be called dendrite cells once they begin the process of phagocytosis), thereby driving and promoting a second crucial step that will create a desired immune (rather than allergic or tolerance) response;

3. the “cassette” system provided by the NALT-active engineered phages can also be used to provide one or more components (such as CpG motifs) that will actively stimulate one or more targeted toll-like receptors (which can be located entirely within the interiors of targeted immune cells, such as TLR9 receptors), in ways that will further promote a desired immune response rather than an unwanted allergic, tolerance or localized response;

4. the phage particles will contain both (i) toll receptor activating and/or other adjuvant-type components, and (ii) a selected antigenic polypeptide sequence, preferably in integral and unitary particles that will hold together, rather than in emulsions or other mixtures that may become separated in ways that can render them less effective;

5. these “cassette”-type phage vaccines will be in particulate form, and will have sizes that are ideal for stimulating phagocytosis by immune cells, which is another important step that can promote desired immune responses rather than unwanted allergic or tolerance reactions; and,

6. by manipulating and using both of two different coat proteins, these cassette phages can incorporate a first foreign protein sequence that has either a small or moderate size, and a second protein sequence having a substantially larger size if needed; either sequence can provide the NALT-active transport sequence that will initiate uptake, transport and processing by NALT cells, while the other sequence can provide an antigenic sequence that will trigger a desired antibody formation response.

In addition to those factors, which come into play after a vaccine has been administered, other important benefits are also provided by this approach, including:

(a) this type of phage system can enable extremely rapid manufacture of huge numbers of phage particles, since the bacterial host cells are not killed as they continuously secrete very large numbers of filamentous phages through their cell membranes;

(b) this system of vaccine “cassettes” will enable the development of greatly improved methods for identifying, testing, and developing different antigenic protein sequences for different candidate viruses, since the entire “cassette” system can remain constant and predictable, gradually accumulating a consistent and predictable body of knowledge around it, with only specific and limited antigen sequences being changed to create different vaccines for different pathogens;

(c) a consistent and predictable “cassette” mechanism, which will change only in the particular antigen sequence inserted into it, can enable greatly accelerated development (including any necessary safety and efficacy testing) of new vaccines each time a new microbial threat appears (such as at the start of the flu season, each year);

(d) by proper use and manipulation of coat protein sequences, this cassette system enables the creation and use of divalent, trivalent, or multivalent vaccines, such as flu vaccines having several different antigenic sequences from different strains of flu; alternately, mixtures of different phage vaccines, each one carrying a specific antigenic sequence (these types of vaccine preparations are often called “subunit” vaccines), can be administered in a vaccine mixture;

(e) chemical treatments also can enable these phage vaccines to carry, on their surfaces, additional antigens or adjuvants, such as oligonucleotides or longer DNA strands, labeling agents, etc.;

(f) since they will be administered nasally, these type of vaccines will be well-suited for inoculating poultry, livestock, and other animals, such as by using a “fogging” device to emit large quantities of the phages in a mist form, into the air being breathed by animals in an enclosure; and,

(g) the bacteriophages being used to provide the cassette/delivery mechanism have a long history of study and use, and are nonpathogenic to animals or plants, since they cannot infect any known types of eukaryotic cells.

Accordingly, the vaccines and vaccine cassettes disclosed herein can provide optimized delivery and adjuvant activities, and can be used with any antigenic polypeptide sequence to provide potent and effective vaccines that can be administered via nasal sprays or similar means.

### BRIEF DESCRIPTION OF THE DRAWINGS

FIG. 1 is a flowchart that shows the major steps for screening a display library containing a very large number of phages, in a manner that will select and isolate those particular phages which demonstrate: (i) efficient binding to NALT cells that are accessible in the nasal sinuses, and (ii) efficient intake and transport of those particular phages, by NALT cells in the nasal sinuses.

FIG. 2 shows the time-dependent passage of two different populations of phages into olfactory bulbs in the forebrains of mice, after administration by nasal spray. The open circles indicate that “wild-type” phages (with no foreign DNA sequences from a display library) passed through the olfactory bulbs with a single early peak. The dark circles indicate olfactory bulb concentrations of phages that contained polypeptide sequences from single-chain variable fractions (scFv) of human antibodies. A double peak was observed. The early peak indicates that some phages passed through quickly, like wild-type control phages. The subsequent peak indicated that other phages passed through more slowly, suggesting that the phage scFv sequences in those phages had some level of binding affinity for either NALT tissue, or olfactory bulb tissue.

FIGS. 3A and 3B show the time-dependent passage of two different populations of phages into circulating blood, in mice. FIG. 3A shows the transport of: (1) wild-type phages without any foreign scFv sequences (“single-chain variable fraction” sequences, isolated from human antibodies), indicated by open circles; and (2) phages containing scFv sequences, indicated by black triangles. FIG. 3B (which uses a vertical scale that is flattened by a factor of 10×) compares the same types of wild-type phages, against phages carrying scFv peptide sequences that had been selected by four successive rounds of in vivo nasal-to-blood transport screening.

FIG. 4 shows a screening test for selecting NALT-targeting phages from a phage display library containing random 15-mer foreign polypeptide sequences in a coat protein. In the first stage, phages that lingered in the olfactory bulb (thereby showing some affinity for olfactory bulb tissue) were harvested, grown in bacterial hosts, and used as the starting population for second-round screening. In the second round, phages were harvested from NALT cells in an animal's windpipe. In the third stage, phages selected by the two prior rounds were incubated with mammalian white blood cells, such as peripheral blood mononuclear cells (PBMCs), to identify phages that bind to the immune cells. In the fourth stage, phages selected by the third round were isolated from the “phagosomes” of mammalian PBMCs, to identify and isolate phages carrying peptide sequences that drove efficient “phagocytosis” (i.e., intake and processing) by the immune cells. In the final stage, the phages that were selected by the successive screening steps listed above are analyzed, to determine the gene and amino acid sequences of the polypeptide fragments carried by those particular phages. Those genes and polypeptide sequences can efficiently drive both: (i) intake and transport by NALT cells, and (ii) phagocytosis and antibody-related processing by white blood cells.

FIGS. 5-10 are photographs or color drawings that will not reproduce well in a published patent application. Therefore, the corresponding pictures have been posted and are publicly available on a website, at www.tetraheed.net/ferguson.

FIG. 5 is a photomicrograph showing fluorescent-labeled phages that were transported into NALT tissues in the nasal regions of mice. These tissue samples were taken 30 minutes after nasal administration.

FIG. 6 is a photomicrograph showing fluorescent-labeled NALT-targeting phages that were transported into immune tissues in lymph nodes in the neck, and into antigen-presenting cells (APCs), two hours after nasal administration.

FIG. 7 is a photomicrograph showing fluorescent-labeled NALT-targeting phages that intensely labeled a minority of the cells in a preparation of human lymphocytes.

FIG. 8 contains panels 8A, 8B, and 8C, which display the results of fluorescent-activated cell sorting of human lymphocyte cells that had been incubated with NALT-targeting phages that had been selected in prior screening tests.

FIG. 9 contains panels 9A, 9B, and 9C, which are photomicrographs created by using fluorescent-labeled antibodies that bind to MHC-1 protein sequences, showing that screened and selected NALT-targeting phages efficiently triggered the clustering of MHC-1 proteins on human lymphocytes. This result indicates that the cells have committed to a desirable cross-presentation of antigen, rather than triggering an undesired allergic, tolerance or localized response.

FIG. 10 is a photograph of the results of a gel-shift assay showing a reduction in electrophoretic mobility, when plasmid DNA was coated onto the surfaces of cationised phage. Since DNA is negatively charged, it clung to the cationised phages (i.e., phage particles that had been treated to create positive electrical charges on their surfaces), creating neutralized DNA/phage complexes with little if any net electrical charge. Since the DNA/phage complexes had little if any electrical charge, they were not driven through the gel by an applied voltage.

FIG. 11 depicts the results of a purification method that used ceramic hydroxyapatite in an affinity column to purify phages. When the concentration of sodium biphosphate was increased, during a series of elution steps, phages emerged in purified form, in the peak that includes elution fractions 61 through 71.

FIG. 12 is a drawing depicting a phage particle that carries a NALT-targeting polypeptide in one type of coat protein, and an antigen sequence derived from a microbial pathogen (or from cancerous cells) in a different coat protein. Exposed DNA strands with a CpG motif (a known type of “pathogen-associated molecular pattern” (PAMP) that can activate TLR9 toll-like receptors) cling to the surface of the cationised phage particles, to help ensure a desired immune response rather than an undesired allergic or tolerance reaction.

Amino acid sequence data also is disclosed herein, for polypeptide sequences that were demonstrated by screening tests to potently drive both: (i) NALT-targeting, intake, and transport activity; and (ii) phagocytic intake and processing by antigen-presenting cells (APCs).

### DETAILED DESCRIPTION

As summarized above, this invention discloses methods for developing and using modified virus particles (which includes phages as well as eukaryotic viruses) having specialized “transport” polypeptide sequences (also called targeting-and-delivery sequences, as described below), as potent vaccines that can be administered to mucosal surfaces of animals, such as in the nasal sinuses and/or the mouth and throat, without requiring needles.

As used herein, “animals” includes humans, and can refer to either an individual or a population, and the term “inoculated” refers to any animal or human (or any population) that has received a vaccine, regardless of the route or mode of administration. To satisfy the claims, a vaccine preparation must be suited for use in at least one animal species, and does not require activity in all species (for example, a vaccine intended for humans does not require activity or efficacy in any other species, and a vaccine intended for chickens does not require activity or efficacy in any other species). The phrase, “A vaccine preparation for delivering antigens to an immune system of at least one type of animal” includes and covers both: (i) a batch of vaccine material in bulk, such as in a bulk container being transported from a manufacturing site to a packaging, distribution, and/or inoculation site, and (ii) a batch or aliquot of vaccine material that is packaged in some type of single-dosage or multiple-dosage form.

The invention also discloses three closely-related compositions of matter, all of which can be created by using the methods disclosed herein. The first composition of matter comprises vaccine “cassettes”, which have been optimized so that the viral genome is ready to have a foreign DNA or RNA sequence inserted into a target insertion site, as described below. This type of “vaccine cassette” particle or preparation does not yet carry an antigenic DNA or RNA sequence that will trigger the production of antibodies; instead, a “cassette” is designed and optimized to receive and handle any antigenic sequence that a vaccine-manufacturing company chooses to insert into a cassette.

In addition to vaccine cassettes, this invention also covers complete vaccine particles, in which a foreign (or heterologous, exogenous, passenger, payload, etc.) DNA or RNA sequence has been spliced into a target (or insertion) site in the viral genome. The foreign DNA or RNA sequence will encode an antigenic polypeptide sequence, such as a sequence derived from a surface protein of a pathogenic microbe. When complete vaccine particles are reproduced, by culturing them in host cells, the foreign antigen sequence will appear in an exposed and accessible location, in copies of a surface protein. As used herein, “surface protein” refers to microbial proteins that are exposed and accessible (usually in multiple copies) on one or more surfaces of a virus or other microbe. “Coat proteins” of phages (and of various other types of viruses) are a class of surface proteins; other types of viruses are surrounded by membrane-type envelopes (usually made of lipid bilayers, taken from one or more membranes of a host cell), and the surface proteins in such viruses are embedded in, or otherwise affixed to, the envelope.

The third composition of matter disclosed herein comprises vaccines that contain “targeting-and-delivery” polypeptide sequences (referred to in the claims as “transport” sequences) that were identified by screening of a phage display library, regardless of whether the vaccine particles are, or are not, phage particles. After a potent targeting-and-delivery polypeptide has been identified (such as the transport polypeptide disclosed herein with sequence data), it can be inserted into various types of viruses or other vaccine components other than bacteriophages, to make enhanced vaccines. For example, a potent “target-and-deliver” polypeptide sequence can be inserted into surface proteins of vaccine viruses that are manufactured by culturing them in eukaryotic cells, such as in bird eggs, caterpillars, insects, mammalian cells, etc. This approach can enable a transport sequence to be incorporated into “glycosylated” viruses and other types of viruses that are formed and/or processed by eukaryotic cells, in ways that bacteria cannot accomplish. This approach is discussed in more detail below, under the heading, “NALT-Targeting Polypeptides In Eukaryotic Viruses”.

As another option, the types of “target-and-deliver” polypeptide sequences disclosed herein also can be inserted into surface proteins in vaccines made from cells, such as killed or disarmed pathogenic microbes that are used to create various types of vaccines. This option also is discussed in more detail below.

Because of how they function, and because of how transport sequences as discussed are identified, isolated, and reproduced, a vaccine cassette (and a vaccine cassette preparation) becomes useful, valuable, and lawful for use as a vaccine, only after a “clonal” isolate has been identified, isolated, and sequenced, using screening tests such as described below, and has been proved and demonstrated to carry a polypeptide sequence that will actively promote the specialized “target-and-deliver” functions described herein. Accordingly, selection and use of suitable screening and isolation steps, leading to identification and purification of clonal isolates that will deliver antigen sequences in vaccines to targeted immune cells, is crucial to this invention.

As used herein, the terms “clonal” and “monoclonal” are used interchangeably, and refer to a population of viruses (which can include eukaryotic viruses, bacteriophages, virions, etc.) that have descended from a single ancestor virus, with all members of the population presumably being genetically identical. Clonal (or monoclonal) colonies of viruses (and of virus-infected host cells) can be obtained by known methods. Typically, a dilute preparation of host cells, briefly incubated with phages or other viruses, are inoculated at moderate density onto one edge of a solid (agar or gel) nutrient that contains an antibiotic, in a shallow dish. The inoculated area is then “streaked” at high speed (creating low density) across the remaining area of the plate, using a tiny wire loop. Since the only cells that can grow on the nutrient in the culture plate are cells that contain an antibiotic-resistance gene carried by the viruses, clonal colonies (isolated from each other, and typically having small round shapes that grow larger as time passes) will arise. A small sample of virus-carrying cells from a clonal colony can be harvested from the culture plate, and those cells can be grown (in very large numbers, but still in clonal form) in liquid or other media. These and other methods for isolating clonal populations are well-known and conventional.

In order to identify clonal phages containing specific polypeptide sequences that will perform the highly specialized “target and deliver” functions disclosed herein, the clonal phage isolates disclosed herein were screened and selected from “phage display libraries” that contained millions or billions of candidate phages, all having different “inserts” containing foreign polypeptide sequences. Any such phage display library may contain several (or possibly dozens or even hundreds) of phages that may function, with varying levels of moderate, good, or excellent potency, as a vaccine cassette as disclosed herein. However, it would be grossly unsafe, illegal, and unethical to use, as a vaccine, an unscreened or even partially-enriched library, which will contain millions or billions of unhelpful and unwanted phages that could provoke potentially severe and dangerous allergic or immune reactions. A safe and effective targeting-and-delivery system for a vaccine can be created only by identifying, isolating, and reproducing one or more specific phages which happen to carry specific foreign polypeptide sequences that will activate and drive a series of desired immune cell responses, as described herein. Accordingly, the claims below contain phrases referring to, for example, compositions of matter such as “a clonal virus (or phage) preparation”, which is distinct and very different from a phage display library.

Terms such as “purified” and “vaccine preparation”, as used in the claims, require attention. They do not require that a purified preparation must contain only certain phages or other viruses, and nothing else. Instead, formulated vaccine preparations are controlled mixtures, having an overall level of purity that renders such a mixture suited for medical or veterinary use, in which a “purified preparation” of clonal phages (or other particles with antigenic protein sequences) is one component. Other components may include carriers and/or diluents (which can be either a liquid or a powder); microbicides or other preservatives or stabilizers; an acid, alkali, or salt; adjuvant-type additives; and, if a vaccine is to be administered to a mucous membrane, one or more mucoadherents. In addition, some viral vaccines contain mixtures of two or three different types of vaccine particles (these are often referred to as divalent or trivalent vaccines), and some bacterial vaccines have even more substituents (for example, two vaccines against pneumonia are referred to as 7-valent and 23-valent).

Accordingly, a vaccine preparation (or a clonal population of phages, other viruses, or cellular microbes, or an intermediate preparation that is created during a manufacturing process) is deemed to be “purified” if the vaccine particles have been processed by one or more purification steps of a type used in vaccine manufacture. Such purification methods, used in manufacturing vaccines and other pharmaceutical or biochemical compounds, are well-known, and include (as non-limiting examples) filtering (which can remove a population of viruses from a population of host cells), centrifugation and other physical methods, methods that use affinity binding, and methods that exploit differing travel rates, settling positions, or other factors that can be exploited by using gels, matrices, or other semi-permeable media (often used in conjunction with voltages, ionic gradients, etc.).

Similarly, “vaccine preparations” are not limited to final and complete formulations. Instead, a “raw” or unfinished preparation containing clonal virus particles is a vaccine preparation, if it will be passed through one or more purification, processing, blending, or other manufacturing steps that will render it suited for use as a vaccine.

It should also be noted that at least some preparations disclosed herein are likely to not need refrigeration or other special handling. This can greatly improve their potential for use in less-developed countries. For example, most bacteriophages and other viruses that are enclosed in coat proteins, rather than lipid membranes, do not contain water as an essential component. Therefore, they can be manufactured and packaged in lyophilized, dessicated, or other dehydrated form, which can endure in exceptionally stable form for long periods of time, even in hot climate conditions. This factor also takes advantage of the fact that “infective viability” of virus particles is not essential, to provide potency and efficacy as vaccines (as evidenced by the fac that many vaccines are specifically made from heat-inactivated or otherwise “killed” viruses).

Certain other terms in the claims also merit attention. For example, certain claims specify, “a polypeptide sequence that is foreign to said viruses (or bacteriophages), and that has been demonstrated by screening tests to promote: (i) uptake of particles carrying said polypeptide sequence into at least one class of immune cells on at least one type of mucosal surface, and (ii) entry into phagosomes of antigen-presenting cells . . . ” In that claim language, the phrase, “foreign to said viruses” refers to polypeptide sequences that are “foreign” with respect to wild-type viruses. Unless otherwise modified, all descendants of a clonal phage line will contain the same “foreign” insert. The fact that the progeny phages will inherit and contain a “foreign” insert from their ancestor does not make an insert “native” or less foreign, with respect to those viruses; instead, if a polypeptide sequence is “foreign” to wild-type phages, then it also is foreign to any progeny in a clonal population.

As used in the claims, the term “promote”, when applied to polypeptide-driven uptake (or intake, transport, delivery, or similar terms) of particles by NALT cells and/or phagocytic antigen-presenting cells (which includes macrophages), requires a level of activity (or efficacy, potency, or similar terms) that allows otherwise identical particles (such as bacteriophages having an assortment of different foreign polypeptide inserts) to be cleanly and clearly separated, isolated, and identified, by means of actual performance in screening tests that rely upon intake of such particles into NALT cells and/or into the phagosomes of antigen-presenting cells. Such screening tests provide a straightforward and functional method that will enable anyone skilled in this art to determine whether (and how strongly) a certain polypeptide sequence can and will promote the intake of such particles, into such cells. The results of such tests can be quantified, if desired, by various methods, such as competitive screening tests (comparable to competitive binding assays), in which a polypeptide sequence of interest can be pitted and tested against another polypeptide sequence, such as in a test that can, if desired, use a mixture of two different phages, at identical concentrations, tested in a single animal or cell culture. The other polypeptide sequence (usually called a comparison or control sample, or similar terms) can be provided by, for example: (i) a random and unsorted population of phages, such as in a phage display library; or, (ii) the polypeptide sequence disclosed herein.

In theory, questions might arise over whether low-level promotion of cellular uptake is actually occurring, if the rates of uptake are only slightly higher than “background” levels. However, in this context, questions about weak or borderline transport-promoting activity will not be significant, and will not need attention, since any vaccine manufacturing or supply company will obliged to disclose information to governmental reviewing agencies that will support claims that a proposed vaccine is potent, and will offer widespread and useful protection in a large majority of a treated population. That requirement, for proven and potent performance, will oblige companies to select and use polypeptide sequences that will strongly and undeniably promote transport of their vaccine particles into NALT cells and/or macrophage cells.

For purposes of quantification, since numerical measuring standards are nearly always useful in research, an arbitrary “benchmark” level of potency is hereby established, at a level of at least 50% of the cell-intake efficacy of the polypeptide sequence disclosed herein, when measured using NALT-related cells or tissues (which may include measurements of “downstream” tissues in animals), or in monocyte, macrophage, or other appropriate cell cultures. This type of “benchmark” standard can be measured by inoculating a population of animals (preferably with at least six mice, rats, or chickens per sample, to obtain statistically-significant results) or cells, with a 50:50 mixture that contains both: (i) a first phage preparation carrying the foreign polypeptide sequence disclosed herein, and (ii) a second phage preparation carrying a second candidate foreign polypeptide sequence that is being tested and measured. If the number of cells or phagosomes that contain phages having the second candidate foreign polypeptide sequence is equal to 50% or more of the number of cells or phagosomes that contain phages having the polypeptide sequence disclosed herein, then the second candidate foreign polypeptide sequence should be regarded as being capable of promoting cellular intake into NALT cells and/or antigen-presenting cells.

Clearly, the ultimate goal of such tests should be to identify polypeptide sequences that can perform even more potently and efficiently than the sequence disclosed herein, to enable the development and use of improved nasally-administered vaccines that are even more potent and effective than vaccines which carry and use the polypeptide sequence disclosed herein. However, since the sequence disclosed herein is believed to be highly and even extremely potent in promoting the specific types of targeting-and-delivery (transport) activities that are of interest in vaccines, that polypeptide sequence, disclosed herein, provides a useful and convenient “benchmark” level, and any other polypeptide sequence that can promote the same types of transport, at a comparative level of 50% or greater, should be regarded and classified as potent and efficacious, even if it is not as potent as the sequence disclosed herein.

### Phage Cassette Design; DNA Insertion Sites

The reference herein to “cassette” systems (or vaccine cassettes, or similar terms) uses a term that is well-known in genetic engineering. Such “cassettes” usually involve plasmids, phages, or other vectors that have been manipulated in ways that allow them to be readily and conveniently modified, by inserting additional strands or segments of DNA or RNA into one or more predetermined locations in such vectors. The term “cassette” arose in the pre-digital era of music, when a cassette player could play a tape containing any musical selection the owner happened to have. Cassettes had standard sizes and mechanisms (several types became popular, initially for audio recordings, and subsequently for video recordings). To use a cassette, an owner merely inserted a tape containing the desired music or video (or a tape that was ready to be recorded onto), into an accommodating machine. Furthermore, the cassettes themselves could be loaded with any audio or video content that the owner or operator chose.

In genetic engineering, “cassette” systems are somewhat different but well-understood. Their principal feature is that they are designed to receive, accommodate, and work with essentially any DNA or RNA sequence that is inserted into them, so long as the insert has a compatible format, as will be understood by those skilled in the art.

While RNA vectors and cassettes sometimes are used, and are widely discussed in the prior art, DNA vectors generally are preferred, for a number of reasons. One important set of reasons arise from two facts: (i) DNA is more chemically stable than RNA; and, (ii) all cell types have mechanisms that actively and continuously degrade strands of RNA, as part of the continuous process of making, using, and recycling messenger RNA (mRNA), which occurs constantly in all active cells. Therefore, DNA vectors are more stable and reliable, since the risk of unwanted chemical and/or enzymatic degradation of DNA strands is lower than for RNA strands.

Another major factor that leads to a preference for DNA vectors and cassettes arises from that fact that whenever an RNA vector is used, some type of “reverse transcription” step (i.e., creation of DNA, from the RNA strand carried by the vector) is almost always required, to create a lasting and inheritable transformation. If an additional required step must be inserted into an already-complex process, it creates another set of potential problems, and reduces the likelihoods and rates of desired outcomes. Therefore, it usually is easier and more reliable to use DNA vectors, unless strong reasons indicate that an RNA vector is better suited for some particular task. The filamentous phages described herein carry DNA, rather than RNA, and that approach is usually preferred, for uses such as described herein.

As a third important factor, DNA vectors tend to be easier to work with than RNA vectors, because there is a broader (and more adaptable and useful) assortment of “restriction endonuclease” enzymes that can be used to manipulate DNA vectors, compared to RNA vectors. As described below, restriction endonucleases that can cleave double-stranded DNA are very useful, in genetic vectors, since they enable a cassette to be provided with several unique insertion sites, allowing foreign DNA sequences to be inserted into specific targeted locations without disrupting any important genes in a vector.

Nevertheless, some types of viruses (often called “retroviruses”) carry RNA rather than DNA, in their genomes; examples include the human immunodeficiency viruses (HIV) that cause AIDS, and the coronaviruses that cause severe acute respiratory syndrome (SARS). Accordingly, although the discussion herein focuses mainly on DNA vectors, for convenience, the teachings herein also can be adapted and applied to vaccines that use retroviruses, and to other types of vectors that carry RNA rather than DNA.

Most “cassette” vectors used in genetic engineering are provided with a genome that has been manipulated to allow “foreign” DNA sequences (which also can be called exogenous DNA, heterologous DNA, passenger DNA, inserted DNA, payload DNA, cargo DNA, or similar terms) to be inserted into one or more specific known target sites. This is accomplished by providing a vector with at least one (and preferably several) unique “restriction” sites, which can be cleaved by “endonuclease” enzymes (such as EcoR1, BamH1, HindIII, etc.). These types of enzymes will cleave a DNA strand only if a certain sequence of nucleotides is encountered. Restriction sites usually require four to six nucleotides, in an exact sequence; as one example, the restriction sequence for BamH1 is G/GATC/C, where the slash marks indicate the cleavage locations on the double-helix strands of the DNA. The BamH1 sequence is symmetric, since the sequence of bases on the other strand of the double helix is the reverse, CCTAGG. The BamH1 enzyme will leave a 4-base “sticky end” on each of the two resulting “cut ends” of DNA, when it cleaves double-stranded DNA. A foreign DNA segment can be given accommodating “sticky ends”, to promote insertion of the foreign DNA sequence into the cleaved vector.

Cassette vectors usually are designed to have several unique restriction sites (such as one site that can be cleaved by EcoR1, another site cleaved by BamH1, and a third site cleaved by HindIII), clustered together in a location that enables a foreign DNA sequence to be inserted into a targeted location without disrupting any genes or other sequences that are important to functioning of the vector. If several such cleavage sites are available, at least one endonuclease almost always will be available that will not inadvertently cleave a foreign DNA sequence being inserted into the cassette. If all of the desired restriction sequences are present in a foreign DNA sequence, the foreign DNA sequence usually can be altered, by replacing one or more 3-letter codons in the foreign DNA sequence with other codons that encode the same amino acid residue (this is enabled by the redundancy of the genetic code, which uses 64 different codons to encode only 20 different amino acids). In vaccines of the type disclosed herein, a typical antigen sequence usually is only about 15 amino acids long, and rarely (if ever) exceeds 30 amino acids. Therefore, “codon swapping” is simple and easy, and a foreign DNA insert that will encode a desired antigen sequence (with any desired selection of codons) can be made rapidly, using automated DNA synthesis machines.

In addition, any gene (or other sequence of interest) in a cassette vector can be flanked by one or more unique restriction sites, to enable the gene to be removed from the cassette vector at an appropriate time. For example, viral vectors usually contain an antibiotic resistance gene (such as a gene that encodes an enzyme that will inactivate ampicillin or tetracycline) or some other type of “selectable marker” gene, to make it easier to isolate and reproduce the vector in bacterial cells. To avoid any questions or concerns about allergic reactions, unwanted antibiotic resistance, or other complications, any such marker gene can be removed, after the basic research has been completed and a vaccine candidate is approaching final testing and actual use.

These and other components and methods that make genetic cassettes easy to handle and use are well-known, and they can be adapted for use in vaccine cassettes as disclosed herein.

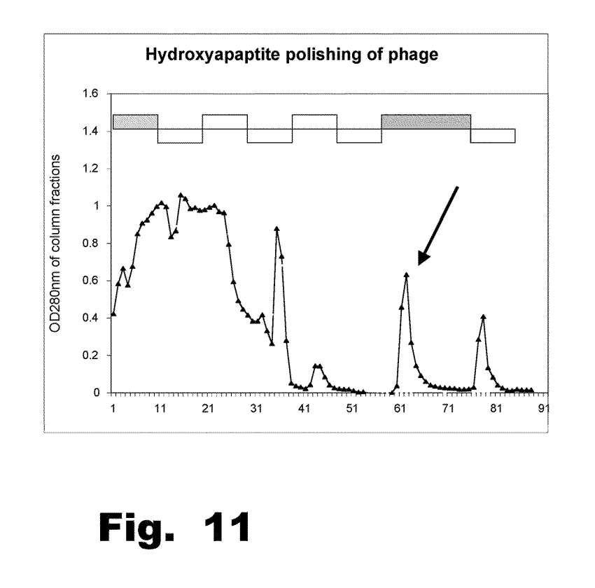

When filamentous phages are used as vaccine cassettes, at least one and preferably several candidate DNA insertion sites preferably should be located in the coding portion of a gene that encodes “coat protein 3” (also referred to as pIII, cpIII, or cp3), which is present (in several copies) at one end of each phage, as illustrated in FIG. 11. This will cause the foreign polypeptide sequence to be inserted into (or added to one end of) the amino acid sequence of coat protein 3, which can carry relatively large foreign protein sequences.

In another preferred embodiment, the phage cassettes also should contain insertion sites in the gene that encodes “coat protein 8” (also referred to as pVIII, cpVIII, or cp8). Over 2000 copies of that coat protein are present in the cylindrical outer shells of filamentous phages.

In yet another preferred embodiment, two different genes that encode coat protein 8 (cp8) can be present in the viral genome, and only one of those two genes, under the control of a relatively weak and/or inducible promoter, will be provided with DNA insertion sites. This can create phage particles with a few hundred copies of a foreign protein sequence, while most of the coat protein 8 subunits have unmodified sequences. This will reduce the “burden” (or passenger load, payload bulk or weight, or similar terms) that the resulting vaccine particles must carry. If all copies of the cp8 subunit in a modified phage vector carry foreign polypeptide sequences, the foreign sequence often must be limited to less than about 10 altered amino acid residues, to avoid hindering reproduction of the phages. By contrast, if only a minority of the copies of the cp8 coat protein carry a foreign insert, the insert often can be substantially longer, such as up to about 15 to 20 amino acids, and possibly more. In either case, the exact length of a tolerable insert will vary, depending on the specific amino acid sequence of the insert.

### Targeting-and-Delivery Components and Aspects

The phage vaccine cassettes disclosed herein can be regarded as comprising a targeting and/or delivery system. Because of the nature of its functions and components, it also can be referred to by other terms that imply an intentionally-designed delivery system (such as, for example, a transport, transfer, carrier, vehicle, ferry, uptake, intake, conveyance, or homing system).

A DNA sequence that is inserted into the genome of a phage cassette, and the foreign polypeptide sequence that will be encoded by the foreign DNA (which will appear in one of the coat proteins of the modified phage) also can be referred to by various terms. For example, such DNA sequences (and the polypeptide sequences they encode) can be referred to as passenger or payload sequences (or components), or as antigen or antigenic sequences. Inserts for vaccines must be antigenic, because of the nature of vaccines; however, non-antigenic sequences can be inserted into a phage cassette for other uses, if desired. For example, a foreign DNA sequence encoding a polypeptide sequence which is not antigenic, but which binds tightly to a known monoclonal antibody preparation or to a particular surface molecule on certain types of cells, can be useful in research or in diagnostic, imaging, or similar work.

If an inserted DNA and/or amino acid sequence is referred to as foreign, heterologous, exogenous, or similar terms, such terms imply that the inserted sequence is not present in the phage, regardless of whether the “foreign” sequence might be present in an animal or human treated by a vaccine. For example, a polypeptide sequence that is “foreign” to a phage cassette can contain a polypeptide sequence that appears on the surfaces of cancer cells, in cancer patients who will be treated by the vaccine, in a vaccine designed to trigger the formation of antibodies (and/or the activation of cytotoxic T cells) that will kill the cancer cells.

As mentioned above, specialized immune cells are present on several different mucosal surfaces, including vaginal and rectal surfaces. Accordingly, vaccine cassettes as disclosed herein can be identified and isolated for any such set of mucosal immune cells, merely by screening for cells that are actively taken into any such cluster or class of immune cells.

However, it has been reported (Kozlowski et al 2002) that in humans, nasally-administered antigens can stimulate stronger antibody responses than vaginal, rectal, or other candidate mucosal routes. In addition, nasal administration can be enhanced and speeded up, merely by inhaling a nasal spray; it does not require any removal of clothes or other time-consuming preparatory actions; and, if a major pandemic emerges, a long line of people can be treated very rapidly (without even requiring any delays for creating or keeping records, if local public health officials deem it prudent to speed up mass distribution as much as possible).

For those and other reasons (combined with the fact that nasal routes can eliminate needles and the problems that accompany needles), the NALT tissues in the nasal sinuses and pharynx area appear to offer an ideal site for administering vaccines as disclosed herein. Accordingly, any references herein to “NALT-targeting” activity are used for convenience, and are intended to be exemplary rather than limiting, to refer to activity in triggering uptake (also referred to as intake, entry into, etc.) by one or more types of specialized immune cells that are exposed and accessible on one or more types of mucosal surfaces.

If desired, specialized bactericidal nozzles for nasal sprays can be used. For example, the surfaces of certain metals (such as silver) can increase the microbicidal potency of alcohol and certain other disinfectants. Accordingly, nasal-spray nozzles coated with silver or other metals can be rapidly and efficiently disinfected by wiping them with alcohol, between uses. Alternately or additionally, a spray nozzle (coupled to a supply tube from a trigger-operated dispensing unit) can be designed with a placement component that would be pressed against the epidermal skin, above the upper lip and below the nose. This would enable two thin tubes at the upper tip of the spray nozzle, spaced roughly a centimeter apart, to inject a small pressurized jet or spray of liquid into both nostrils, without touching anything inside the nostrils.

As mentioned in the Background section, prior versions of intranasal vaccines have not lived up to their potential, because of various limitations and shortcomings. Among other factors, adverse reactions (such as inflammation of the olfactory bulbs, after intranasal administration) have been observed in some cases (Van Ginkel et al 2000); and, better intranasal adjuvants are needed (Lang 2001; Levine 2003). There has been too much variability, among test animals in populations that have been treated, making it difficult or impossible to determine reliable parameters such as optimal dosing regimens. In addition, lab animals sometimes respond to nasally-administered vaccines in ways that are different from human responses, leading to still more unwanted variables and complications.

The specialized targeting-and-delivery components and aspects of the mucosal vaccines disclosed herein are believed to overcome those problems, and offer a greatly improved class of mucosal vaccines, in which a combination of useful components will work together synergistically to provide improved vaccines that can outperform any known mucosal vaccines in the prior art.

Furthermore, the cassette approach of this invention can enable the screening and development of various different sets of phage cassettes, with each set being optimized for a particular species. For example, the first set of NALT-intake screening tests described herein used mice. If desired, a second set of phage cassettes can be screened and isolated in rats, using the same or similar screening approaches; a third set of phage cassettes, screened and isolated in chickens, for use in chickens; and so on. Those types of screening tests can be repeated in any species of interest (such as in species that are important in farming or food supply, in veterinary or medical use, etc.), up to and including humans. Similarly, these types of phage cassettes can be optimized for various wild species that are being endangered by viral or other epidemics.

Based on factors of evolution, homology, species similarities, and cross-reactivity, it is highly likely that phages isolated in one species will also function effectively in closely-related species. As examples, phage cassettes that are screened and isolated in mice are likely to function efficiently in rats, rabbits, and other rodents; phage cassettes that are isolated in chickens are likely to function efficiently in turkeys or other poultry; and phage cassettes that are isolated in monkeys or chimpanzees are likely to function efficiently in humans and other primates.

It also should be recognized that the immune systems of all mammals face very similar pressures, demands, and needs, since all mammals must be able to fight off heavily overlapping types of microbial pathogens. As a result, there are unusually high levels of similarity, homology, and cross-reactivity among some components of mammalian immune systems, even among widely different classes of animals (such as rodents, and primates). This is shown by, for example, the extensive homology that exists among toll-like receptors in species as different as mice and humans. Accordingly, phage cassettes such as disclosed herein, and adaptations of the screening methods disclosed herein, will provide immunology researchers with powerful tools for analyzing and quantifying various types and degrees of overlap and cross-reactivity, between various components and cell types of the immune systems of different types of mammals. During such research, it is likely that some particular phage cassettes will show high levels of vaccine-type potency among different classes of mammals (such as between mice and humans), while other phage cassettes will show lower levels of cross-reactivity.

Accordingly, whenever desired, isolation of a preferred phage cassette for use in a particular species of interest (and, in some cases, in a particular strain, race, or other group of interest, such as in specific strains of mice or rats that are widely used in immunological research, or in humans who live on continents or islands, where different types of pathogenic microbes pose the most severe threats to health) may be able to provide even more potent and efficient carrier phages, for use in a particular species or other ancestral group. While some phage cassettes are likely to emerge that can provide good vaccine potency among all humans, other more specialized phage cassettes may be able to provide even higher levels of potency among people whose ancestors lived and evolved among a semi-localized and particularized set of pathogens.

For practical reasons, since solid tissues can be harvested easily from mice, the screening tests described herein initially used mice, to screen for phage intake into NALT tissues, and for transfer from NALT tissues into other tissue types (such as olfactory bulb tissue). In subsequent screening tests involving monocyte cells (white blood cells which are the precursors of macrophage cells), human cells were used, partly because it is easier to obtain large quantities of white blood cells from humans than from mice, and also because the goal of this research is to move rapidly toward vaccines that can and will be used in humans.

With regard to isolating phage cassettes that are optimized for human medical use, two points should be noted. First, in many situations, collecting phage isolates that have passed a screening test will not require solid tissue samples; instead, isolates can be collected by using blood and/or lymph, by means that only require aspiration (removal of liquid) using a hypodermic needle. Second, if a compelling need ever arises for obtaining solid tissue samples from humans, such samples can be obtained from people who have been declared brain-dead after a major trauma (such as a fatal automobile accident, cardiac arrest, massive stroke, etc.), or who have lapsed into a terminal coma at the end-stage of a disease such as cancer. Fatally-injured people who are “brain-dead” are often kept alive for hours, days, or even weeks, using respirators and intravenous feeding, to enable the harvesting of organs or other tissues for transplant purposes. That is an accepted and respected medical practice, and it provides grieving relatives with a sense that a terrible loss of a loved one was able to save other lives. Accordingly, similar practices can used to enable harvesting of NALT or other tissues, from humans, if a compelling need ever arises.

### Useful Steps; Complete and Optimized Delivery

Two different and potentially conflicting concepts must be understood and kept in balance, to understand this invention.

The first concept is this: if a properly screened and isolated vaccine cassette can accomplish even a single crucial targeting-and-delivery step, it can provide an important and useful advance over the prior art. Accordingly, any such advance merits recognition and coverage for what it has accomplished, and some of the claims below focus on phage vaccine cassettes that were screened and isolated because they were able to accomplish a single specific step that is useful or crucial in provoking a desired immune response to a vaccine.

The second concept, which points in a different direction but which must also be taken into account, is this: if a single targeting-and-delivery vaccine cassette can accomplish not just one or two steps, but an entire series of steps, all of which will increase the likelihoods and rates of desired immune responses in the largest possible number of animals or people among a treated population, then that type of enhanced multi-step targeting-and-delivery system provides an even more important and useful advance, compared to a targeting-and-delivery system that was selected by only one or possibly two screening steps.

This factor arises from how vaccine preparations are used, in the real world. To prevent the spread of disease, they must be administered in large numbers, to large populations. Because of the stochastic, probability-dependent, bell-curve nature of large biological populations, and no one can predict with certainty how any particular person or animal will respond. Therefore, any company, agency, institution, physician, or other health care provider that distributes or administers vaccines to animals or people must accept and assume the responsibility to develop, procure, and use the best, most potent, most effective candidate vaccine(s) that are available, to provide optimal benefits.

This obligation is especially important, since most diseases pose the greatest risks to people who are not entirely healthy, and not in optimal condition to fight a disease. As examples, very young children do not have fully-functioning immune systems; the immune systems of elderly people gradually grow weaker, less agile, and less effective; and, the immune systems of many adults have been compromised and weakened by various injuries, infections, and diseases, and in many cases by smoking, suboptimal diet, excess weight, alcohol or drug abuse, and other problems.

Accordingly, targeting-and-delivery phage vaccines should (and ultimately must) be identified and isolated, not just by using a single round of screening, but by using a succession of several different screening tests, where the starting population for each screening test is obtained from candidates that performed well in other types of screening tests. This has been accomplished by the methods disclosed herein, and if desired, it can be repeated in screening tests that are limited to one particular species (such as humans, for example), to isolate phage vaccine cassettes that will be truly optimized for that particular species.

The sequence of screening steps that were used in the initial demonstration of this invention, and the immune cell responses that were screened for, by those particular tests, can be summarized as set forth below. Because of the nature of these tests, in some cases a single screening test (which culminated in harvesting and isolating phages from a specific type of tissue or cell) required that several distinct cellular steps had to be completed successfully, in order for phage isolates to be isolated from the targeted cells or cell compartments.

1. The first screening test isolated phages that had been taken into NALT tissues (which line the nasal sinuses and upper throat area, and which are present on the surfaces of breathing passageways in rodents), and which were then transported to one or more types of “downstream” (or “second-stage”) tissues or cells. Accordingly, this test identified and isolated phages that successfully completed each and all of the following steps: (i) intake into NALT cells, presumably via endocytotic or other cell-surface receptors; (ii) release of the phage by the receptor, after a phage/receptor complex had entered a NALT cell; (iii) secretion of the phages by the NALT cells, into some type of cellular junction that delivered the phages to one or more types of “downstream” cells; and, (iv) intake of phages that had passed through NALT cells, into one or more types of “second stage” cells or tissues.

To complete this screening test, the “second stage” tissue was harvested, and the membranes of the cells were dissolved, using a detergent-type “lysis buffer” that dissolves lipid membranes of cells, without damaging the coat proteins of viruses. That step released the contents of the cells, allowing the phages to be extracted. The isolated phages were then reproduced in E. coli bacteria, to provide a starting population for subsequent screening tests.

2. A second screening test (which used “enriched” populations of phages that had been screened for NALT intake and transport, as described above) was used to isolate phages that triggered binding to (and/or intake into) macrophage cells. As mentioned in the Background section, the term “macrophage cells” as used herein includes monocyte cells, which are precursor cells that become macrophage cells after they pass through a capillary wall, leave the circulating blood, and enter the lymph fluid in soft tissue.

This screening test began with sampled human blood. The red blood cells were removed, and the semi-purified white blood cells were processed by a surface-binding step, to isolate monocyte (macrophage) cells, which have unusual surface-adhering molecules that enable monocytes to grip a capillary wall and permeate through the capillary wall, to reach the lymph fluid. The purified monocyte (macrophage) cells were incubated with phages that had been fluorescently labeled, and the cell/phage mixture was processed by “cell sorting”, using a machine called a flow cytometer, to isolate monocyte (macrophage) cells that were strongly labeled by fluorescent phages. This was done by setting the controls of the machine so that only the top 3% of the cell population was isolated, based on strength and intensity of the fluorescent signal from a cell/phage complex.

The membranes of the isolated highly-fluorescent monocyte (macrophage) cells were dissolved, viable phages were extracted, and the harvested phages were reproduced in E. coli, to provide starting populations for subsequent screening tests.

3. The third screening test (which used phages that had already been selected by the screening tests described above) isolated phages that triggered phagocytosis (i.e., entry into macrophage cells). This test was carried out by using several stages of centrifugation, to isolate phagosomes (i.e., intra-cellular compartments enclosed within their own membranes) from macrophage cells, obtained from human blood by the surface-binding selection process mentioned above. The cells were incubated with phages that had passed the prior screening tests, and were then processed using a mechanical homogenizer. The homogenizer broke apart cells, without breaking the phagosomes (which are much smaller than cells). Intact phagosomes were isolated by (i) a first mild centrifugation, which formed a pellet of intact cells and nuclei, which were discarded; and, (ii) a second stronger centrifugation, which pelletized the phagosomes. The phagosome pellet was resuspended in liquid, then the membranes were dissolved by a lysis buffer, and phages contained within the phagosomes were harvested, and reproduced in E. coli. Accordingly, to be present in intact and functional phagosome compartments, the isolated phages had to activate and participate properly in three sequential cellular processes: (i) binding (as a “ligand”) to a phagocytic receptor, on a surface of a macrophage cell; (ii) intake of the bound receptor/ligand complex into the macrophage; and (iii) separation of a completed and functional phagosome, from the outer cell membrane.

### Enhancements and Adjuvant Activities

In a preferred embodiment, the phage cassettes disclosed herein can include (and/or can be supplemented by) enhancing components that can promote additional desired responses. Such cellular responses might include, for example: (i) activation of one or more types of toll-like receptors (TLR's), to help ensure that an immune response leads to desired immune response, rather than an undesired allergic or tolerance reaction; (ii) activation of a desired Th1 and/or Th2 response; and, (iii) activation of a desired MHC-1 and/or MHC-2 response. These types of enhancements can enable vaccines to be optimized for either of two different usages: (1) inducing an antibody-producing “humeral” response, for fighting off microbial pathogens; or, (2) inducing other cell-mediated responses, such as for killing cancer cells, or for removing other types of cells or materials (such as, for example, beta-amyloid plaques in the brains of people suffering from Alzheimer's disease).

Some of those enhancements and/or adjuvant features, activities, and advantages of these vaccines arise from the inherent properties of the phage cassettes. As one example, these phages appear to have an ideal size for triggering intake into NALT cells, and into phagosomes within macrophages.

Other enhancements, which can include components that can be regarded as adjuvants (or as having adjuvant-like activity), can be provided in any of several ways, such as by using one or more of the following approaches: (i) adjuvant, adjuvant-like, or other enhancing components can designed and incorporated into phage cassettes, so that such components will be integral and inseparable features of all vaccine particles; (ii) adjuvant or other enhancing components can be covalently or non-covalently bonded to phage particles (which presumably will occur after final assembly of completed vaccine particles that carry inserted foreign DNA sequences and antigenic polypeptide sequences), so that the adjuvant or enhancing components will remain securely bonded to the vaccine particles, until phagocytic or other cellular or enzymatic processes take over and begin dismantling a vaccine particle; and/or, (iii) adjuvant or other enhancing components can be coupled to the phage cassettes by other means, such as by ionic or hydrogen bonding.

As used herein, terms such as adjuvant, adjuvant-like activity, or enhancing components are intended to refer to any component, activity, or other trait or feature that will either: (i) increase the likelihood (or rates, probabilities, yields, etc.) of triggering desirable immune responses, in members of a large treated population; and/or, (ii) reduce the amount of a vaccine preparation that must be administered, to provoke desired immune responses in some target fraction or percentage of a treated population. In interpreting such phrases, it should be kept in mind that some of the vaccines disclosed herein will be designed to help an organism defend itself against an infection, while other vaccines will be designed to help fight cancer or kill other unwanted cells, or to dissolve or neutralize plaques or other unwanted materials. Accordingly, components or traits that can activate one or more types of toll-like receptors (TLRs), or that can preferentially promote MH1 or MH2, Th1 or Th2, or other types of responses, are included within terms such as adjuvants or enhancing components, as used herein.

The ability to incorporate adjuvant components into the vaccine particles themselves, or to use covalent, hydrogen, or ionic bonding to couple components to vaccine particles, can eliminate or minimize a number of problems that have plagued the prior use of adjuvants that are merely stirred into a liquid or slurry mixture. In any such mixture, vaccine particles can separate from adjuvant additives, and the two types of components can migrate in different directions, inside a body, in ways that can reduce the efficacy of the adjuvant, and therefore of the vaccine. Accordingly, by developing and optimizing phage particles into unitary vaccines, which have adjuvant as well as antigen components incorporated into or bonded to the same particles, a set of problems that has plagued and limited vaccines in the past can be avoided.

These approaches also enable improvements in quality control, which poses a major concern in vaccine manufacture and safety, for a number of reasons. “Manufacturing tolerances” must be extremely tight when human lives are at stake, especially in countries where potential lawsuits and liability are major factors that often control business decisions (and that typically impede and hinder major innovations). Quality control problems increase whenever biological (rather than purely chemical or mechanical) manufacturing is used, and quality control problems increase even more when time pressure is important, as in vaccine manufacture (for example, every year, when flu season approaches, public health officials would prefer to wait and delay, for as long as possible, to study what is happening among various candidate flu strains in various populations, before finally selecting the strains that will be incorporated into a flu vaccine that will be used that year). Quality control will become even more difficult yet critical, if a need arises to manufacture hundreds of millions (or even billions) of dosages very rapidly, as may be required if a “bird flu” strain emerges that can be transmitted human-to-human, or if a strain of HIV/AIDS emerges that can be transmitted by insects.

The use of phage vaccines that can be manufactured in huge quantities in a matter of hours, using in vitro culture of bacterial cells (or other types of eukaryotic, glycosylating, or other cells that can be grown in cell culture), can make quality control issues and problems simpler and more manageable, compared to prior manufacturing methods, such as incubating vaccines in bird eggs (which normally must incubate for weeks).

Accordingly, the coat proteins of phage vaccines as disclosed herein can be chemically treated (or modified, enhanced, or similar terms), to enable them to carry or deliver additional antigens, adjuvants, or other useful molecules. For example, if phage particles are “cationised” (i.e., treated with agents that will impart a positive electrical charge to their surfaces), then relatively short strands of DNA, called oligo-nucleotides or oligo-deoxy-nucleotides (abbreviated as ODN's) will cling to the phages (since DNA strands are negatively charged). This will allow DNA segments having (for example) “CpG motif” sequences to be affixed to the surfaces of the phages. As described in the Background section, in DNA strands with CpG motifs, large numbers of unmethylated cytosine residues are positioned adjacent to guanidine residues. These are recognized by mammalian immune systems as a “pathogen-associated molecular pattern” (PAMP), which can activate toll-like receptors, to promote immune response rather than allergic or tolerance responses. Accordingly, DNA strands having CpG motifs can be synthesized and affixed to the surfaces of “cationised” or otherwise treated phages, using ionic and/or hydrogen bonding, to increase the efficacy and potency of the resulting vaccine particles.

Alternately or additionally, certain amino acid residues in proteins can be chemically treated, by known reagents, in ways that will covalently crosslink other compounds to the proteins. As an example, the side chain of each lysine residue in a protein has a reactive primary amine group, at the end of a four-carbon chain. The accessibility and hydrophilic (water-soluble) nature of such amine groups allows certain known reagents (such as isothiocyanate, or bis(sulfosuccinimidyl)suberate) to react with lysine residues, in ways that will create covalent crosslinking bonds with other compounds. Since covalent bonding generally is stronger than ionic and/or hydrogen bonding, such reagents can be used to covalently bond adjuvants, secondary antigens, or other potentially useful compounds to the surfaces of phage particles in vaccines, if desired.

Using such means, phage vaccines as described herein can be used to carry, into the phagosomes of targeted antigen-presenting cells, molecules or substances (which can be referred to by terms such as payloads, passengers, supplements, enhancers, adjuncts, etc.) that normally would not be efficiently internalized by such cells, or that normally would not potently activate a desired immune response in a large fraction of a treated population. Examples of such molecules or substances include: (1) small antigenic epitope molecules; (2) labeling or imaging molecules (also called trackers, tracers, or similar terms), which can be useful in research, diagnostic medicine, and other situations; (3) small and/or soluble antigen molecules that are only weakly immunogenic unless attached to a larger molecule; (4) adjuvant molecules, such as short DNA strands having CpG motifs, or other agents that can activate toll-like receptors; and, (5) plasmid DNA, which in some cases may be able to provoke gene expression, leading to useful polypeptides within (or possibly secreted by) targeted cells. Those are non-limiting examples of compounds that can be useful if incorporated into, or affixed to, phage vaccines having targeting-and-delivery capabilities as disclosed herein.

Screening for Polypeptide Sequences that Drive NALT Intake

As described in the Background section, published Patent Cooperation Treaty patent application WO 2003/091387 described a method for carrying out an in vivo screening process, using the long sciatic nerve bundle in a rat leg, to identify, select, and isolate certain particular phages (out of millions or billions of candidate phages, in a phage display library) that will be efficiently taken into nerve fibers (by endocytotic receptors on the neuronal surfaces), and that will be transported within the nerve fibers, toward the main cell bodies of the nerves. This screening method required that any phages that were actually selected, in the screening test that was used, had to actually and effectively perform both of those two different and distinct functions, by emplacing candidate phages at a first location near the knee, and by harvesting phages from a different location near the hip.

Subsequently, the inventor herein realized that an analogous type of in vivo screening test, using a phage display library administered by nasal spray, can be used to identify, select, and isolate certain particular phages (out of millions or billions of candidate phages, in a display library) that will efficiently target and enter NALT cells in the nasal and throat region.

That task has been completed successfully, and DNA and amino acid sequence data, for the best-performing isolated clonal phage that was identified and isolated by such screening tests in mice, is provided herein. Accordingly, the work done to date in mice demonstrate the efficacy of this approach, and similar screening tests, using the same or similar types of phage display libraries, can be done with any other species of interest, by using the same or similar steps.

The particular steps used in the mice tests are not claimed or asserted to be optimal; instead, certain steps were taken because the inventor herein had done similar work for other purposes previously in his career, and therefore was already familiar with certain types of tests. Nevertheless, the complete set of steps that emerged and evolved, during the course of this research, were shown to work effectively. Now that this approach have been disclosed, experts and researchers who study these disclosures can adapt and improve these particular screening steps, for use with various species. Accordingly, provided that the goals and results remain essentially the same (i.e., to identify phages that will drive NALT uptake and phagosomal intake into macrophages, leading to strong immune response, for use in vaccines or related immunological research), the exact details of similar screening tests that may be developed or used in the future are not essential to this invention.

Furthermore, since the goal of such screening tests is to use a series of tests to identify phages that happen to carry foreign polypeptide sequences that will activate and drive several different steps, the sequence of such screening tests is not crucial. For example, to minimize the number of research animals that must be used, any in vitro screening tests that only require white blood cells can be completed first, and enriched populations of phages identified by the in vitro tests can then be screened by in vivo tests, using intact animals.

It also must be recognized that once a high-performance phage that can function as a vaccine cassette has been identified, either for any particular species or for some range of species (such as a first phage cassette that has been optimized for rodents, a second phage cassette that has been optimized for humans and primates, a third phage cassette that has been optimized for birds, etc.), additional screening tests will no longer be necessary, and the selected phage cassette(s) can simply be reproduced (and can be “tweaked” and further improved and adapted, if desired).

Accordingly, the screening tests developed and used for in vivo testing in mice used the following steps:

1. During prior research that had occurred over a span of years, before this invention arose, the inventor had identified various reagents and methods that helped this work proceed more rapidly and efficiently. Two such reagents are worth noting. First, the inventor used a compound called FITC, which uses fluorescein as a label and isothiocyanate as a linker, which will form a covalent bond that links a fluorescein label to an amine group (such as on a lysine residue) on a protein. The FITC labeling agent enabled rapid analysis of tissue slices under a fluorescent microscope (i.e., a microscope that uses ultraviolet or other short-wavelength light as a light source). It also eliminated the need for expenses and delays that would have occurred if other types of labeling had been used that required incubation with antibodies, specialized enzymes, or other reagents.

Second, the inventor used a mixture of parabenzoquinone and paraformaldehyde, as a tissue fixative. This allowed the exposure time for the fixative reagent to be reduced, which helped preserve the structure of various molecules, and avoided the creation of excessive crosslinking bonds, which otherwise can interfere with analysis.

2. Since the inventor, a medical school professor who specializes in CNS physiology in mammals, had previously done other types of research that involves tracking the travel of various things through brain cells and tissues, he was already familiar with various problems and obstacles that can arise in such research. Therefore, he decided to do a series of preliminary tests, to find out: (i) where a diverse phage library would go, if administered nasally to mice, and (ii) how rapidly phages would appear in (and be removed or cleared from) various types of tissues within the brain. Since he had seen prior results and reports (involving other, earlier research) indicating that some types of compounds, when inhaled by mice, will be taken into the olfactory bulbs, and will pass through that portion of the brain before being taken elsewhere, he decided to include olfactory bulbs as one of the tissue types that were tested for phage concentrations, at various time intervals after nasal administration.

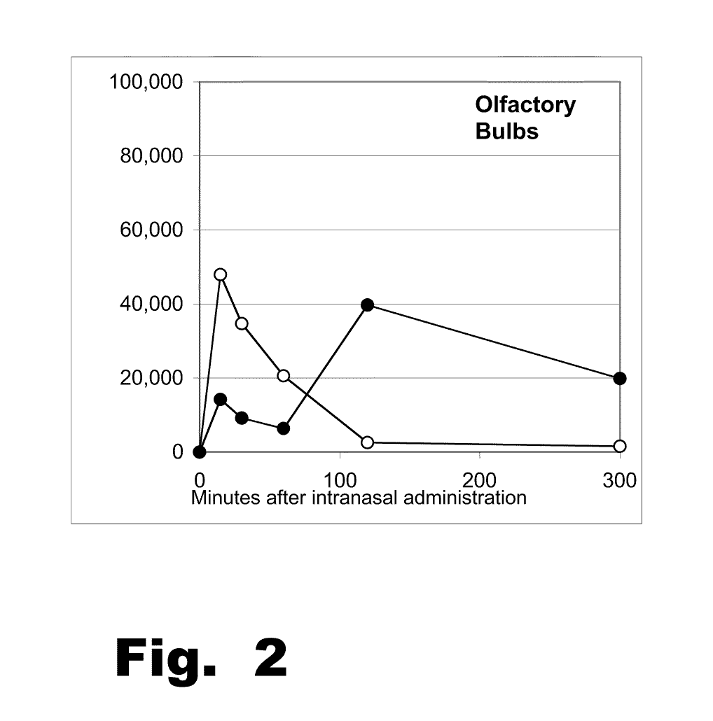

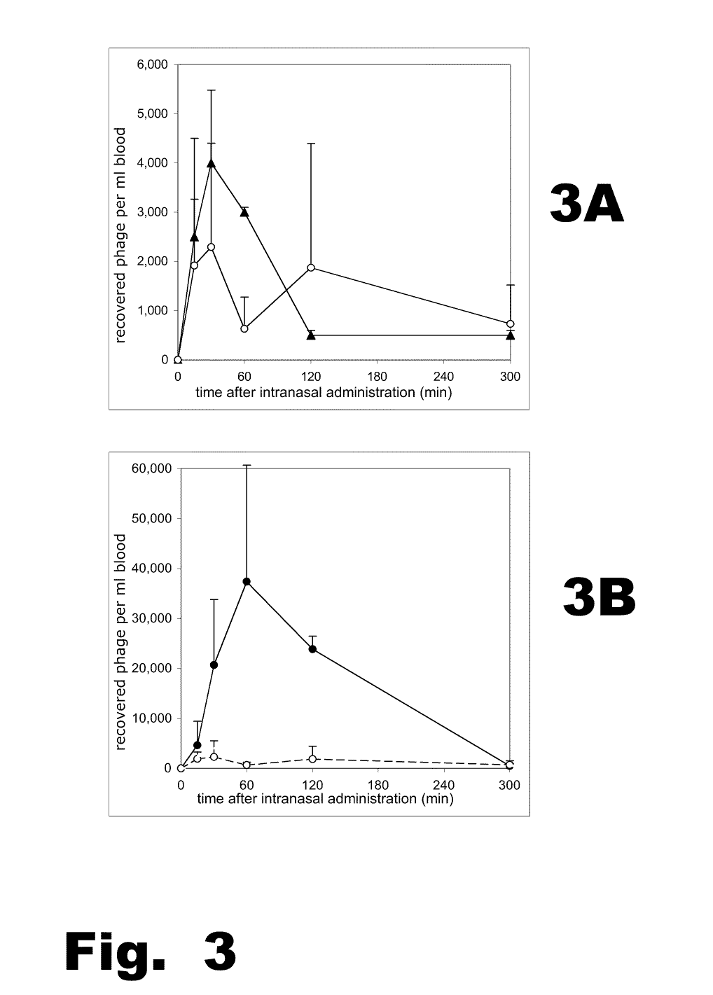

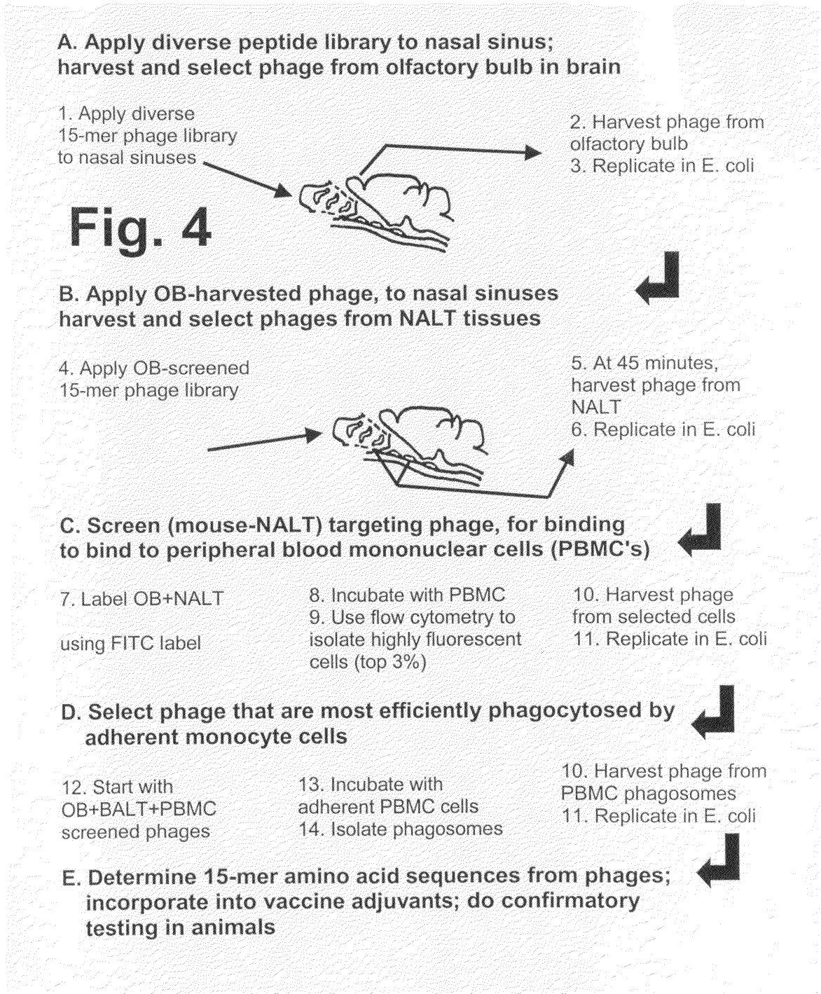

3. The initial tracking and timing tests were performed using fluorescent-labeled “wild type” phages with no foreign gene sequences. The results, indicated by the open circles in the graph in FIG. 2, indicated a peak of high phage concentration in olfactory bulb tissue at the earliest testing time, in mice sacrificed 5 minutes after phage administration. That peak passed fairly quickly, as indicated in FIG. 2.

4. A second series of tests using olfactory bulb tissues was carried out using a phage display library (prepared by Cambridge Antibody Technology) containing scFv gene sequences from human antibody genes, inserted into coat protein 3 (which is present in several copies at one end of a phage particle). The results, indicated by the dark circles in FIG. 2, indicated an early first peak, which coincided in time with the single early peak of the wild-type phages. Unexpectedly, a second peak was seen at 2 hours after phage administration, which had not been present at 1 hour or at earlier test times.

After considering the possible causes for a second peak, the inventor concluded that it most likely reflected some type of low or moderate affinity binding, between: (i) the foreign antibody-derived sequences carried by some of the phages in the phage library, and (ii) the tissues in the mouse brain. These types of binding reactions are occurring as part of a complex and ever-changing series of interactions between several types of fluids and several types of membranes, within and around the olfactory region of a mouse or rat brain. While a detailed analysis of those tissue and fluid types is not necessary for understanding the current invention, it is important to realize that after carefully considering all relevant factors, the inventor realized that the types of low or moderate affinity binding reactions he had observed could be exploited and utilized, by means of time-sensitive in vivo screening tests that could identify phages with NALT-uptake activity.

5. A first round of in vivo screening tests was carried out, using nasal administration of a phage library containing a diverse set of inserted DNA sequences encoding random 15-mer polypeptide sequences in coat protein 8 (which is present in over a thousand copies, in the cylindrical shells of the phages). Either 30 minutes or 60 minutes after administration, animals were sacrificed (n=10 total), and about 20 to 30 ml of saline solution was injected into the aorta, to rinse unattached phages out of the vasculature. Tissue was harvested from the olfactory bulb portion of the brain, and the cells were incubated with a lysis buffer, which dissolved the cell membranes and released viable phages. The harvested phages were reproduced in bacteria, with the help of a tetracycline-resistance gene carried elsewhere in the phage genome (the scFv phage library similarly contains an ampicillin resistance gene). Samples from both sets of animals were pooled together, to provide an enriched starting population for the second round of screening.

6. In the second round of screening, the enriched phage population (containing 15-mer inserts in coat protein 8) were administered. After 45 minutes, the mice (n=10) were sacrificed and perfused with saline solution. NALT tissues (which are visible elongated lumps, which flank both sides of the midline along the bottom of the windpipe) were harvested. The cell membranes were dissolved by lysis buffer, and harvested phages from the NALT cells were replicated in bacteria cells.

7. The NALT screening process was repeated, using phages selected in the first round of NALT screening as a starting population, to further enrich for NALT-targeting phages.

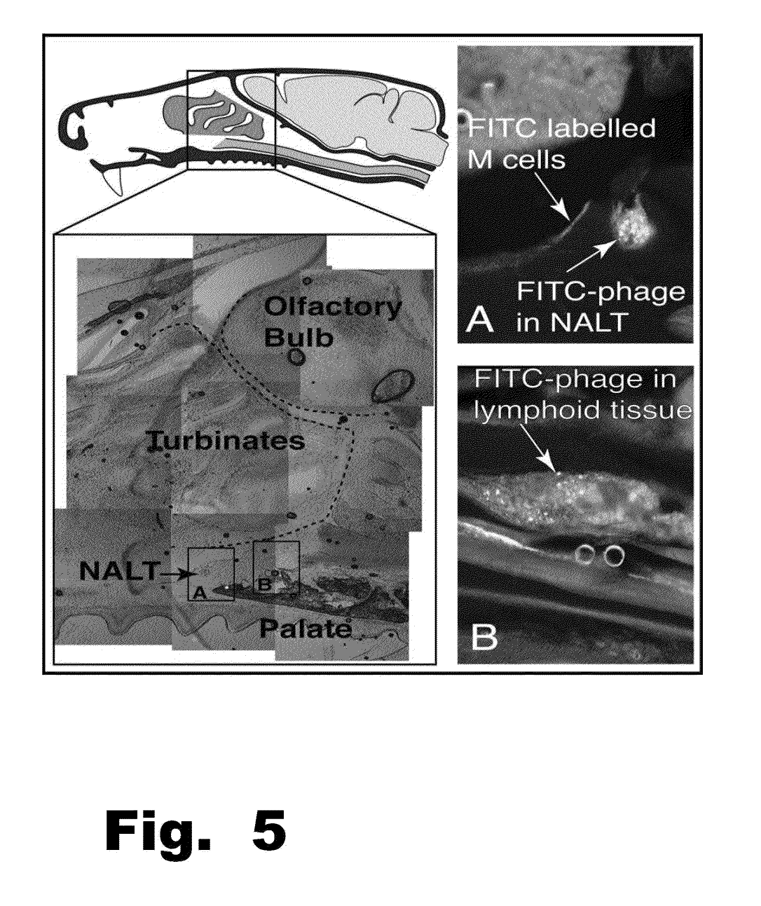

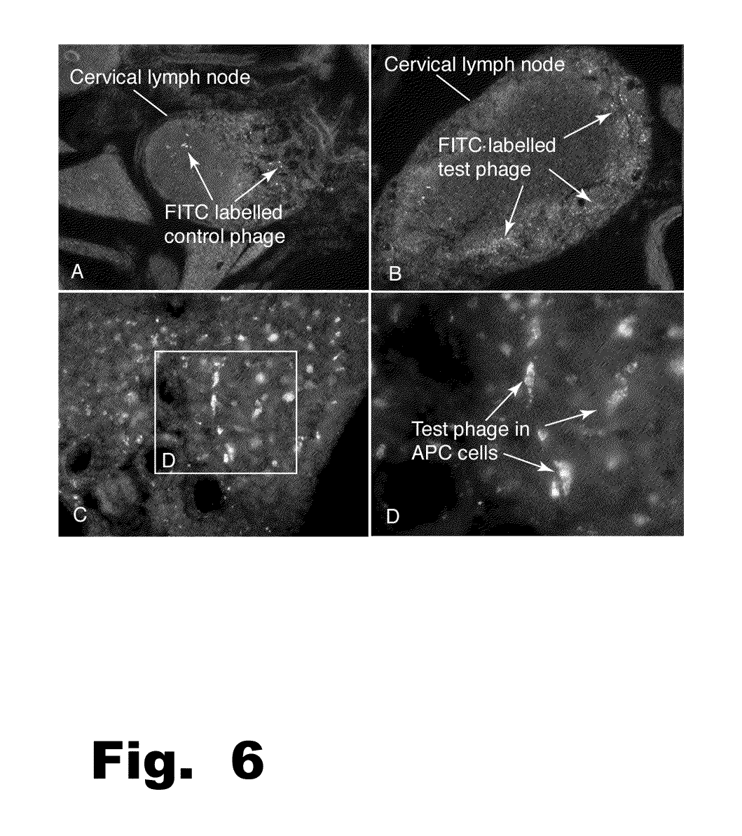

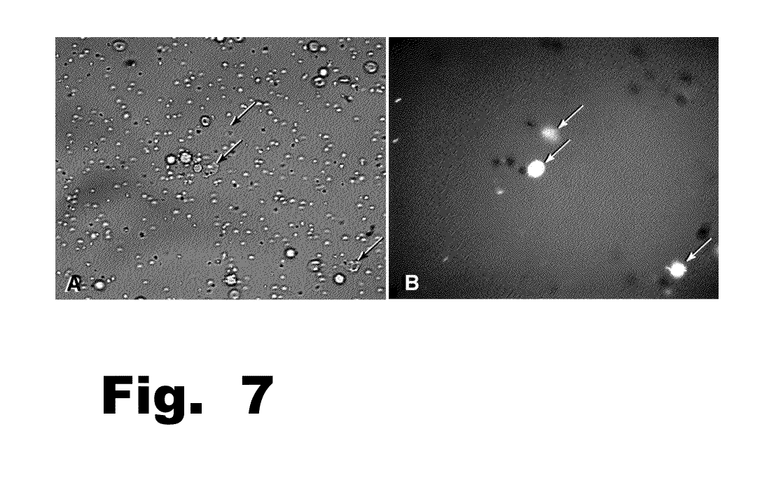

The ability of those screened and selected phages to actually target and bind to NALT cells, and subsequently to be transported by the immune system into lymph nodes, is confirmed by the photomicrographs in FIGS. 5 and 6. Phage preparations that were screened and isolated as described above were replicated, and fluorescently labeled using FITC. The labeled phages were then administered to mice, in nasal sprays. After 30 minutes or 2 hours, some of the mice were sacrificed, and perfused with a mixture of parabenzoquinone and paraformaldehyde. Tissue sections were obtained, and photographed with a fluorescent microscope. The large picture in FIG. 5 indicates the region of tissue that was photographed. The two smaller pictures (which show up more clearly in color photographs posted on a website, www.tetraheed.net/ferguson) clearly indicated the presence of various clusters of fluorescent-labeled phages that had been transported to NALT tissues.

In mice that were sacrificed after 2 hours, the lymph nodes that service the NALT tissues in the windpipes of the mice were harvested and photographed. The photographs in FIG. 6 clearly indicate that the phages selected by NALT screening (as described above) were indeed transported to those lymph nodes.

Screening for Macrophage Binding and/or Intake

Additional screening tests, which began with 15-mer phage populations that already had been selected by NALT screening tests in mice (as described above), were used to identify phages that were actively taken into, and processed by, macrophages. Human macrophages were used, for two reasons: (i) sufficient quantities of macrophages can be obtained more easily from human blood, than from mouse blood; and, (ii) the goal was to identify phages that can lead to actual vaccines for human medicine, rather than merely creating research tools for use in rodents.

A crucial processing step that is carried out by macrophage cells, when antibodies are generated in response to a pathogen or vaccine, involves phagocytosis (i.e., the intake of a solid particle into a cell). As described in the Background section (and in numerous textbooks and review articles), phagocytosis involves four sequential steps, all of which must occur for the complete process to succeed.

In the first step, a pathogen or vaccine must bind to (and activate) a phagocytic receptor, on the surface of a macrophage. Using a standard term that applies to cell surface receptors, any molecule that will bind to and activate such a receptor is called a “ligand”. Accordingly, the in vitro screening steps described herein were used to identify specific phages, from among a large library of candidate phages, which happen to carry polypeptide sequences that act as “phagocytic receptor ligand” sequences.

In the second step, the receptor-binding reaction must activate a membrane-altering process, to trigger formation of finger-like membrane extensions by the cell. These membrane extensions will flank and then surround the receptor/ligand complex, enabling the receptor/ligand complex to be taken into the cell.

In the third step, the membrane “pocket” containing a receptor/ligand complex grows larger, and is drawn deeper into the cell, with the aid of additional “organelles” having their own membranes (such as endosomes and lysosomes). The membranes of those organelles merge with the membrane of the phagosomal pocket that is being formed, to enlarge the phagosomal membrane and speed up the process.

In the fourth step, the phagosome disengages from the outer membrane of the cell, to form a cxomplete and intact phagosome (i.e., a discrete compartment with its own membrane), which encloses a particle such as a microbe or a vaccine.

Once the phagosome has been formed, the macrophage uses specialized processes and enzymes to partially digest the microbe or vaccine, in a way that creates relatively short polypeptide sequences, typically about 15 amino acids long. Those short polypeptide sequences, from a partially-digested microbe or vaccine, are “mounted” on either MHC1 or MHC2 proteins, to form an antigen/MHC complex, which is transported to the external surface of the macrophage cell. When that occurs, the macrophage becomes an “antigen-presenting cell” (APC). It will deliver its surface-mounted antigen polypeptide (from the microbe or vaccine particle) to a “B cell”, which will perform the next steps in creating antibodies that will bind to microbes having the same antigenic polypeptide sequence.

Since phagocytosis is a multi-step process, two different screening tests were used, to ensure that the screened and selected phages would be highly potent and efficient, in initiating and enabling all of the steps in the process. If desired, the first phagocytosis-related screening test might be eliminated, since: (i) the second screening test will screen for the desired final result, and (ii) successful completion of a desired final result implies and even requires that any necessary earlier steps also must have been completed, successfully.

However, the initial screening test is not difficult; it merely requires an initial incubation, followed by flow cytometry using an automated machine. In addition, since vaccines and immune responses involve “stochastic” (probability-dependent) events that will occur in large and varied populations, steps should be taken to ensure that the most potent and efficient candidate phage(s), from a large phage library, will indeed be identified and selected. Therefore, if an additional screening test can increase the likelihood that the most potent and efficient targeting-and-delivery phage (for use in vaccine cassettes) will be identified and isolated, then that screening test should be regarded as useful and desirable, even if not strictly necessary.

Accordingly, a “first phagocytotic” screening test was performed, using a phage population that already had been screened and selected for NALT-targeting activity, as described above. To perform this screening test, human blood was sampled and loaded into tubes, on top of a buffer solution called “Lymphoprep” (sold by Nycomed Pharma, Oslo, Norway). When centrifuged using the manufacturer's instructions, RBCs will pass through the buffer and can be removed and discarded, while WBCs will remain on top of it. The white cells were harvested, washed twice in Dulbecco's phosphate-buffered saline (PBS), and placed in plastic tissue culture flasks.

As mentioned in the Background section, the white blood cells of interest are called “monocytes” when they circulate in blood. They have special surface-adhering molecules that cause them to grip and cling to the internal surfaces of capillary walls. That clinging process is crucial, in enabling these cells to pass through a capillary wall and enter the lymph fluid (the watery fluid that moves slowly through soft tissues). After a monocyte cell leaves the circulating blood and enters the lymph, it swells to a larger size, and is called a macrophage.

Therefore, by exploiting the unusual “surface-adhering” activity of monocytes, it is possible to isolate monocytes from a much larger population of mixed white blood cells. To perform that process, the semi-purified WBC population was placed in plastic tissue culture flasks. After 30 minutes of incubation, any cells that did not cling to the plastic surfaces of the culture flasks were rinsed away, and discarded. The adhering cells were gently scraped off, using a rubber spatula, and were resuspended in a buffer solution. They were then incubated with fluorescent-labeled phages that already had been screened for NALT-targeting activity as mentioned above.

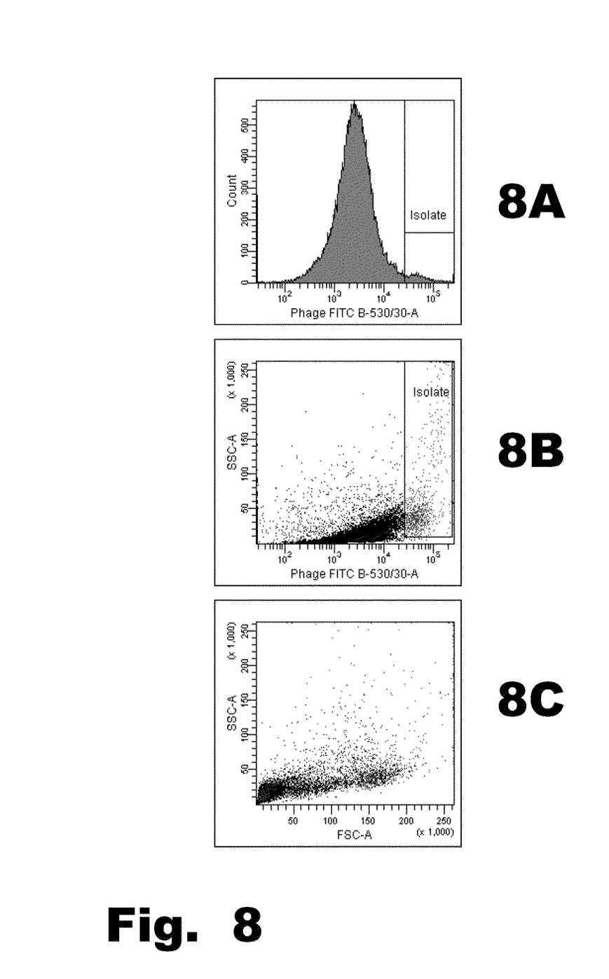

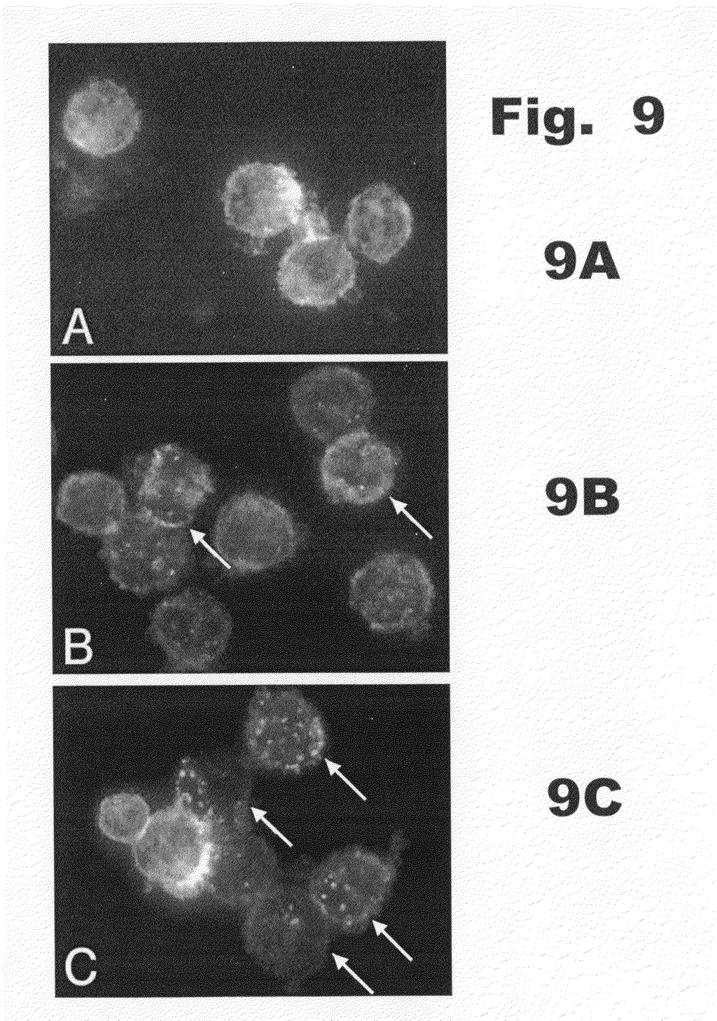

After incubation (to provide time for phages to bind to phagocytic receptors on the surfaces of the monocyte cells), which can be followed if desired by stirring, shaking, or similar processing to rinse away and remove any unbound phages, the monocyte/phage mixture was processed by “fluorescent-activated cell sorting” (FACS). In this process, the cells (suspended in a clear liquid) pass rapidly through a very thin glass tube, in a machine that uses an ultraviolet or similar light beam to activate the fluorescent labels bonded to the phages. A photo-detector (which detected the emission wavelength of the FITC label on the phages) was used to activate a tiny jet of liquid, injected into the flow passageway, each time a highly fluorescent cell passed through the glass tube. That control mechanism caused highly fluorescent cells to be sent to a special collection chamber, while cells with lower levels of fluorescence were sent to a discard bin. The controls on the flow cytometer were adjusted so that only 3% of the monocytes were selected; this is indicated by the rectangle on the right side of FIG. 8A.

In this “first phagocytotic” screening test, it did not matter whether phages entered the cell interiors, or merely clung to the cell surfaces. However, the test did required substantial binding to occur. Therefore, this screening test selected phages that happened to be carrying polypeptide sequences that function as ligands that bind tightly (with high affinity) to phagocytotic receptors on the surfaces of monocyte (macrophage) cells.

The selected monocytes (and their associated phages) were treated by lysis buffer, to dissolve the cell membranes and release the phages. The phages were replicated in bacteria, and were used as the starting population in yet another round of screening, as described below.

FIG. 5 (which includes FIGS. 5A and 5B) provides visual confirmation that the flow cytometry screening test performed as intended. To obtain those photographs, phages selected by the flow cytometry screening were incubated with a preparation of white blood cells. The white cell population contained a relatively small number of monocytes and macrophages, mixed with other non-macrophage lymphocytes. FIG. 5A shows large numbers of white blood cells, under normal visible light (the smaller cells are mainly platelets). FIG. 5B is a photograph of the same cells in the same pattern and position, at a fluorescent wavelength. It shows that fluorescent-labeled phages became bound to only a few of the large cells, which presumably and apparently are monocytes and/or macrophages, while the labeled phages did not bind to the other types of lymphocytes. These results confirm that the isolated phages were selective, and bind with high affinity only to certain targeted types of white blood cells. The “grayscale” photographs submitted with this patent application are not as clear and vivid as the color photographs, which are available at www.tetraheed.net/ferguson.

Screening for Intake into Phagosomes

Since the flow cytometry screening test (described above) tested only for binding of phages to macrophage cells, a final screening test was performed, to isolate phages that were efficiently taken into phagosomes (i.e., discrete organelles with their own membranes) inside macrophage cells. This test was deemed necessary after prior tests that screened for neuronal transport (as described in PCT application WO 2003/091387) indicated that phages which became very tightly bound to endocytotic receptors, on the surfaces of neuronal fibers, apparently were not released properly by the receptors, and were not transported efficiently within the neuronal fibers.

To carry out the phagosomal screening test, another set of PBMC's that adhered to the plastic walls of culture flasks was prepared, as described above. These cells were contacted by a population of phages that already had been screened for NALT uptake, and for macrophage surface binding. After incubation with the monocyte cells, unbound phages were washed off and removed, and a liquid that contained trypsin (a protein-digesting enzyme) and ethylamine-diamine-tetra-acetate (EDTA, which enhances trypsin activity) was added, to detach the cells from the plastic surfaces of the flasks. This trypsin treatment step also served to digest and render non-infective any phage adhering to the outer surfaces of the cell, thereby reducing any risk that phages isolated from phagosomes might be contaminated by phages that did not promote phagocytosis. The harvested cells were suspended in fresh media, centrifuged, and resuspended.

The cell/phage preparation was then homogenized, using 10 strokes of a mechanical plunger device known as a Dounce glass-glass tissue homogenizer. Breakage of the cells by the homogenizer ruptured the outer membranes of the cells, in a way that did not destroy the phagosomes and other organelles (which are much smaller) contained inside the cells. The homogenate was centrifuged under conditions that pelletized the cell nuclei and unbroken cells, while leaving the phagosomes (with any uptaken phages) in the supernatant. The supernatant was then centrifuged at a higher speed for a longer time, to pelletize the phagosomes. The supernatant was discarded, and pelleted phagosomes (with internalized phages still contained inside them) were resuspended in fresh buffer. Lysis buffer was used to dissolve the phagosomal membranes without damaging the phages. This released phages, which were replicated in E. coli cells.

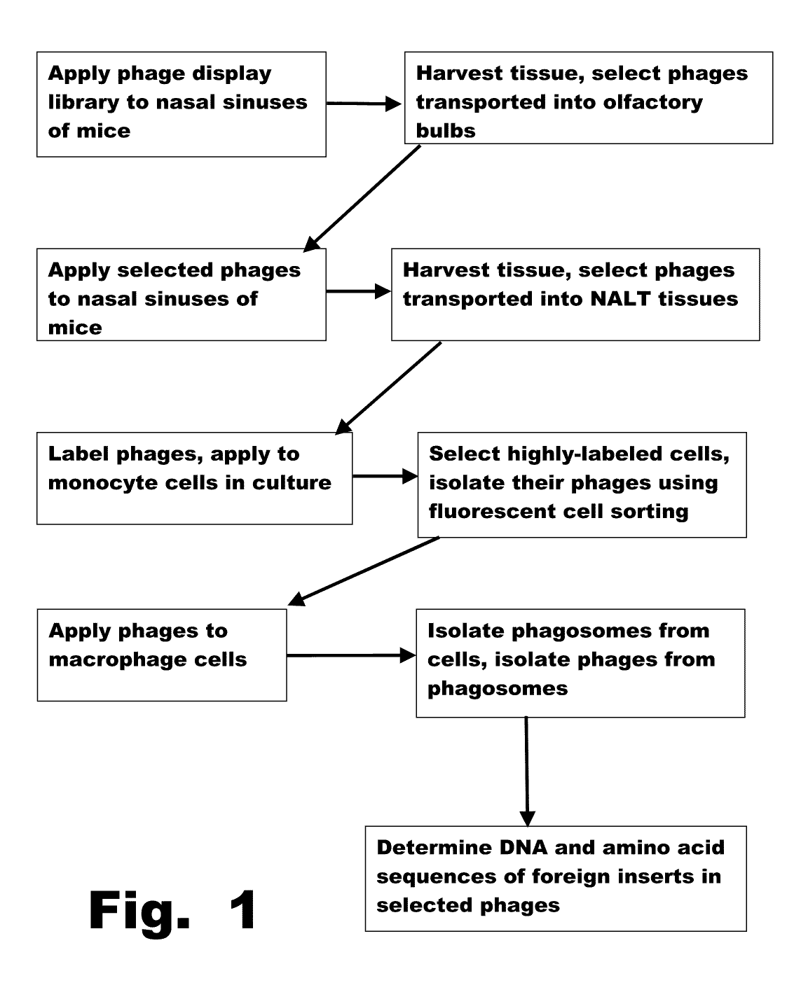

Taking all of the foregoing screening stages into account, a combined synergistic screening process, as listed above, is summarized in the flowchart of FIG. 1. Several clonal phage colonies containing 15-mer foreign polypeptide sequences were isolated by that process, and those phages were sequenced, to determine both: (1) the sequences of the 45-base foreign DNA sequences carried by those phages, and, (2) the amino acid sequences of the 15-mer foreign polypeptide sequences phage carried by those phages. The DNA and polypeptide sequence data are contained and provided within this application.

It also should be noted that screening and selection of a phage display library, using methods such as described herein, can become a very useful tool in studying, analysing, and utilizing various aspects of phagocytosis, including identification of new and additional classes of phagocytic receptors. While a number of classes of phagocytic receptors are already known (such as lectin receptors, Fc receptors, and complement receptors, as mentioned in the Background section and as reviewed in articles such as Aderem and Underhill 1999, Jutras and Desjardins 2005, and Blander 2007), other classes and types are likely to exist, and merit attention. Accordingly, the methods described herein can be used to screen phage display libraries (including phage display libraries that may not have been previously screened at all, or that may have been screened using methods other then the NALT-related screening described herein), to identify additional receptor types of receptors that can trigger phagocytosis.

Once this research pathway becomes better recognized and understood, identification of phagosome receptor ligands for specialized cell types may be used to target efficient delivery of payloads to particular cells, or to stimulate or otherwise modulate various types of phagocytic processes that are important in development, disease, or other processes or problems. For example, stimulation of enhanced phagocytosis of beta-amyloid peptides by microglial cells, in brain tissue, may be able to provide or improve various types of research and/or therapy, in Alzheimer's disease; this concept is discussed in more detail in articles such as Gelinas et al 2004. Similarly, in a process called “Wallarian degeneration”, Schwann cells phagocytose the myelin proteins that form the sheaths of neuronal fibers, (e.g., Hirata et al 2002); and, olfactory-ensheathing glial cells phagocytose the axons of olfactory cells that have been replaced (e.g., Wewetzer et al 2005). Accordingly, those processes can be better studied and understood, and likely exploited for diagnostic or treatment purposes, by screening phage display libraries by methods such as disclosed above, to identify and isolate specific phages and polypeptide sequences that will bind to and activate specific phagocytotic receptors that are not yet adequately understood, but that are likely to be present on the surfaces of various types of specialized cell types.

### Methods and Components for Toll Receptor Activation

As briefly discussed in the Background section, if a vaccine preparation can activate one or more types of “toll receptors” (also referred to interchangeably as toll-like receptors, abbreviated as TLR's), on or in macrophage cells, the vaccine can be much more efficient in provoking a desired immune response. Accordingly, the phage cassette vaccines disclosed herein can be designed in ways that will strongly activate one or more types of toll receptors.

In particular, phage cassette vaccines can be designed and assembled in ways that will activate specific toll receptors that are present inside macrophage cells, rather than on the cell surfaces. This can help increase the safety of vaccines derived from such vectors, since it can avoid unintended activation of toll receptors on the surfaces of macrophage cells, which otherwise might increase the risks of triggering or aggravating allergic or other unwanted responses in some recipients.

Cell-internal toll receptors that are known at the present time are believed to include TLR3, TLR7, TLR8, and TLR9. A large and rapidly growing body of published reports have identified DNA sequences containing CpG motifs (mentioned above) as among the agents that are known to activate internal TLR9 class of toll receptors. Accordingly, DNA sequences contain CpG motifs can be “woven into” the single-stranded DNA genome of phage cassettes as described herein. Alternately or additionally, relatively short DNA segments (called oligo-deoxy-nucleotides, or ODN's) can be affixed to the surfaces of phage particles, using either covalent or ionic/hydrogen bonding as described above, to trigger the activation of TLR9 receptors.

In addition, this type of controllable targeting of specific types of toll receptors is believed to be capable of creating vectors that can drive and steer the immune system toward creating either: (i) a “humeral” response, involving antibodies and B cells; or, (ii) a “cell-mediated” response, involving activated T cells without any substantial involvement by antibodies or B cells. It is believed and anticipated that this type of “guiding” system that targets certain types of toll receptors can be used, in phage cassette systems as disclosed herein, to create two different but important classes of vaccines:

(1) vaccines that will provoke antibody-producing humeral responses, for fighting off microbial pathogens; or,

(2) vaccines that will provoke specifically targeted cell-mediated responses, for killing cancerous cells and for dissolving or otherwise removing or moderating certain other types of harmful or dangerous cells or materials, such as plaques or other deposits.

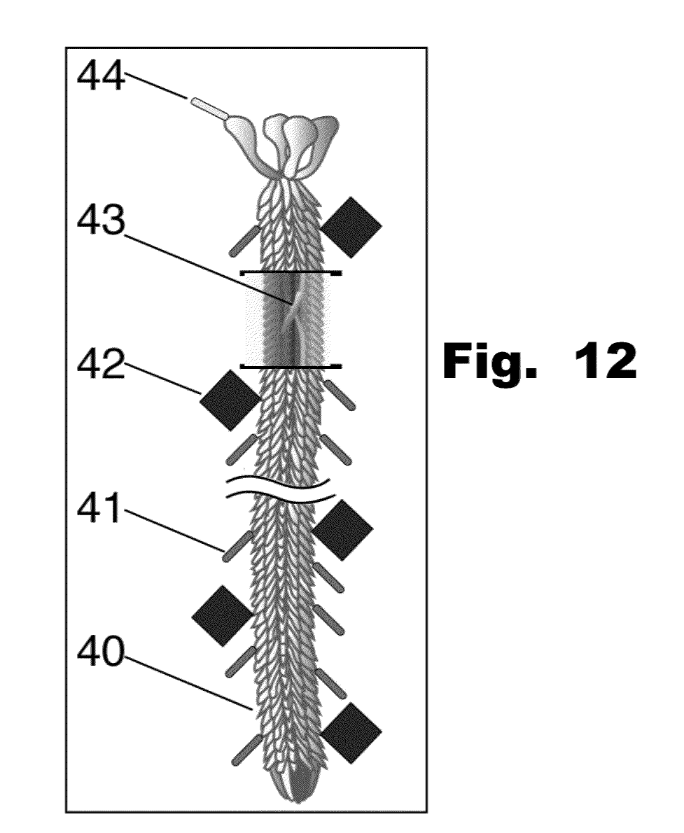

FIG. 12 is a schematic depiction of a cassette-type phage particle 40, with various elements indicated by callout numbers. Callout number 41 indicates a NALT-targeting peptide that will stimulate phagocytosis by human monocytes; callout number 41 indicates another attached molecule that has a different desired function, such as a fluorescent label, a compound that will provoke toll receptor activation, etc. Callout number 43 is a DNA strand carried by the phage, which can contain (for example) a strong CpG motif, to activate toll receptor 9. Callout number 44 depicts an antigenic protein that has been incorporated into coat protein 3 (CP3) of the phage; several copies of the CP3 proteins are present at the end of a phage filament, and at least one (and presumably all) of those copies have been modified to include a relatively large foreign polypeptide sequence.

With regard to toll receptors, it should be noted that the use of CpG motifs, in DNA strands carried inside filamentous phages, is believed to represent an advance in methods and reagents for exploiting toll receptor activation. When a phagocytic cell internalises and processes a bacteriophage, DNA sequences that were carried and hidden inside the phage will be exposed, and can begin to stimulate TRL9 receptors, in ways that can initiate and/or increase an immunostimulatory cascade that will lead to an enhanced immune and antibody response. Filamentous phages provide a different technology platform for developing and delivering CpG sequences, as immune adjuvants, compared to other candidate delivery vehicles. In other settings, ODNs typically must be synthesized to be resistant to DNAase activity by the host cells. In the phage cassettes as disclosed herein, immunostimulatory CpG motifs carried within the phage filament will be shielded and protected from DNAase inactivation, by the phage coat proteins. However, those phage coat proteins will be removed, after a phage vector has entered a macrophage or other phagocytic cell.

Furthermore, in contrast to synthetic ODNs, which become increasingly expensive to manufacture and purify as the number of base pairs in the oligonucleotide increases, filamentous phage DNA is not constrained by size. Therefore, it becomes entirely practical to incorporate CpG or similar immunostimulatory DNA sequences having lengths greater than 200 base pairs, into phage genomes. This will allow, for example, repeating CpG motifs, as well as permutations that will combine CpG sequences from different microbial species and/or that will have several different recognized patterns combined in ways that can be conveniently produced by phage cassette systems. For example, Klinman et al 2004 describes three distinct subclasses of CpG motifs, designated as D-type, K-type, and C-type motifs. Accordingly, phage cassettes as disclosed herein will enable researchers to determine whether the presence of two or more such patterns can lead to additive or even synergistic levels of potency and efficacy, when incorporated into vaccines.

In summary, by considering the components and factors described above, and by using gene and polypeptide sequences that have been identified (by screening tests) as having strong levels of NALT-targeting activity, experts who are skilled in the art, after studying and considering the teachings herein, will be able to understand how to create NALT-targeting phage cassettes and vaccines, and how to enhance the adjuvant properties of such cassettes and vaccines in desirable ways, such as by modifying the cassette genomes to include stimulatory or inhibitory CpG motifs, or other components of interest. These cassette systems will be able to receive and incorporate selected foreign DNA sequences (such as, but not limited to, sequences that encode antigenic proteins from pathogenic microbes), and they will be able to deliver the foreign DNA sequences to specific targeted classes of phagocytic cells, in ways that have not been possible under the prior art.

### Collection, Assembly, and Coordination of Components

In understanding the types of phage cassette vaccines disclosed herein, it is important to understand how each of the component parts contribute, and interact with each other. These components and their roles can be summarized as follows:

(1) the phage cassette will provide components that can be referred to as carrier, vehicle, transport, targeting, or delivery components, or by similar terms. To function in this manner, the cassettes must provide, in an exposed surface-accessible location, at least one polypeptide sequence that has been shown to provide at least one (and preferably all) of the following activities:

a. targeting-and-delivery activity that will promote intake of vaccine particles into specialized cells that are exposed and accessible on one or more mucosal surfaces. As mentioned above, because of several factors, NALT cells (in the nasal and throat region) offer a convenient, rapid, efficient, and preferred route for mucosal administration. However, other mucosal routes (involving vaginal, rectal, or other surfaces) can be used if desired. Accordingly, references herein to “NALT-targeting” activity are used for convenience, and are exemplary rather than limiting, and refer to activity in triggering uptake by one or more types of specialized immune cells that are exposed and accessible on one or more types of mucosal surfaces.

b. targeting-and-delivery activity that will promote the intake of vaccine particles into phagosomes, inside macrophage cells. As described above, phagocytic processing by macrophage cells (which is a multi-step process) is a crucial step in generating a desired antibody response to a vaccine. Therefore, a coat protein of a phage cassette preferably should contain one or more exposed and accessible polypeptide sequences that will actively trigger binding to (and activation of) phagocytic receptors, on the surfaces of macrophage cells. Such polypeptide sequences can be referred to as phagocytic (or phagocytotic) ligands.

(2) In addition to the targeting-and-delivery components mentioned above, other components (which can be referred to as adjuvant components, enhancing components, or similar terms) also be incorporated into such vaccines, if desired, as optional components or improvements. As one example, the phage cassettes disclosed herein can contain DNA sequences having CpG motifs (either carried within the genomes of the phages, or affixed to the surfaces of the phage particles), which can activate targeted toll-like receptors in ways that will increase the likelihood of a desired immune response (which will depend on the type of vaccine that is being administered).

(3) One or more foreign gene sequences will be inserted into the genome of a phage cassette, into targeted insertion sites that are properly positioned within the coding sequence for a viral coat protein. These insertion sites will contain unique sequences that can be recognized and cleaved by at least one (and preferably several) restriction endonuclease(s). In vaccines designed to trigger antibody production to help fight a microbial pathogen, such as a virus or bacteria, the inserted foreign DNA sequence will encode an antigenic protein sequence that is normally found on the surface of the pathogen. By placing such antigenic protein sequences into phage cassettes that will specifically target and deliver the antigenic polypeptide sequences to NALT cells and then to macrophage cells, increased efficacy and potency for such vaccines can be achieved, and nasal administration of these vaccines can offer an effective route of administration, not just for humans, but for large numbers of animals as well (notably including poultry).

Now that this new class of vaccines has been described as set forth above, a reader should review the comments in the two itemized lists set forth in the “Summary of the Invention” section, above. One list is numbered as items 1 through 6, while the other list is numbered as items “a” through “f”. Those skilled in the art will reach a better understanding and appreciation of those listed factors and potentials, and will be able to create more effective use of those factors and potentials, once they have studied and recognized the concepts and components summarized in this “Detailed Description” section.

### DNA Vaccines

It has been reported that microparticles can be used to deliver, into a mammalian body, DNA strands that can be expressed into foreign polypeptides, by means of normal cellular processes. This approach, called “DNA vaccination”, is reviewed in articles such as Jilek et al 2004. Under the prior art, it has not been highly efficient, and it has not become a widely-used method of vaccination.

However, certain aspects of the current invention can be adapted and utilized in ways that will substantially increase the efficacy and potency of the “DNA vaccine” approach. In particular, single-stranded DNA segments (such as from phages that carry ssDNA) or double-stranded DNA segments (such as from bacterial plasmids) can be affixed to the surfaces of phage particles as disclosed herein, using either covalent bonding, or ionic/hydrogen bonding, as mentioned above. By using such methods, strands of ssDNA and/or dsDNA can be affixed to the surfaces of particles that can be manufactured inexpensively in large quantities, and that can potently and efficiently deliver surface-affixed DNA strands into specific targeted cells, such as: (i) into NALT cells and macrophages, if the phages that were identified and isolated by screening tests such as disclosed herein; or, (ii) into other cell types, if the phages were identified and isolated by screening tests that select for uptake into those particular cell types.

One such treatment pathway that merits special attention involves so-called “Th1” responses, as described in more detail in the next section.

Vaccines that Selectively Activate Th1 or Th2 Responses

As mentioned in the Background section, “T helper” cells will respond to different types of triggering events in ways that will “commit” the cells to converting into either of two distinct classes of cells, referred to as Th1 or Th2 cells. There are several ways to distinguish between Th1 and Th2 cells, depending on how they are activated, and what they do after they are activated. Importantly, those known differences also offer potential ways to manipulate and control vaccines, in ways that can “steer” or “direct” T helper cells in either of those two directions when desired.

The distinctions between Th1 cells versus Th2 cells are described in review articles such as Moingeon 2002, Knutson et al 2005, and Burrows 2005. While other articles such as Rosloniec et al 2002 point out that the distinctions are not always entirely clear, and that apparently paradoxical responses are sometimes observed, relevant reports generally indicate and agree upon the following:

(1) T helper cells commit to the Th1 pathway when a messenger molecule called interleukin 12 (IL-12) is present. The Th1 cells then begin producing gamma interferon, interleukin 2, and tumor necrosis factor alpha.

(2) by contrast, T helper cells commit to the Th2 pathway when IL-4 is present. The Th2 cells then begin producing more IL-4, as well as IL-5 and IL-10.

(3) Th1 cells are involved in what are often called “cytotoxic T cell” (CTL) responses, also called “cell-mediated” responses. These can be important in fighting cancer, and in fighting some types of chronic, lingering microbial diseases. However, cell-mediated responses also are involved in autoimmune diseases, and can create severe problems in some cases.

(4) by contrast, Th2 cells are involved in systemic responses that use B cells to create antibodies (these are also called humoral responses).

By taking those differences between Th1 versus Th2 pathways into account, and by also considering other articles and teachings in this field, it is believed and anticipated that steps can be taken that will enable phage cassette vaccines as described herein to be manipulated in ways that will trigger either: (1) Th2 responses, when desired, such as for creating antibodies that will fight off microbial pathogens that typically cause short-term infections, such as flu viruses; or, (2) Th1 responses, when desired, such as in vaccines for treating cancer or other disorders, or for treating some types of lingering infections. As stated in Guy et al 2005, “one can now choose adjuvants able to selectively induce T helper Th1 and/or Th2 responses, according to the vaccine target and the desired immune response.”

As examples, DNA sequences with CpG motifs that will stimulate Th1 (“cell-mediated”) responses are described in articles such as Krieg et al 1998, while other DNA sequences with “suppressive” CpG motifs that will steer the immune system toward Th2 (antibody-generating) responses are described in articles such as Ho et al 2003 and Shirota et al 2004. Accordingly, these types of relatively short DNA sequences will merit attention, as enhancers and adjuvants that can be incorporated into, or affixed to the surfaces of, phage cassette vaccines as described herein.

In addition, timed coadministration of selected interleukin molecules or certain other cytokine molecules, and/or other types of adjuvants as described in articles such as Guy et al 2005, can also be used to help steer an immune response in a desired Th1 or Th2 direction.

A primary reason to vaccinate is to “prime” the immune system, so it can recognize a future invasion and respond as rapidly as possible. As part of this priming activity, when the immune system launches a Th2 type of response, it generates “memory” B cells that effectively “remember” the vaccine antigens. This process involves a complex mechanism, wherein a large variety of responsive B cells with reshuffled short DNA sequences are generated, which will encode a variety of newly-created variable fragments that are incorporated into new types of antibody molecules. Subsequently, the immune system identifies particular B cells that happen to be making antibodies that efficiently bind to the invading microbe. Those particular B cells are stimulated to reproduce rapidly, causing the enlarged population of selected B cells to secrete large numbers of their antibodies. Then, after an infection recedes, the number of those particular B cells drops off greatly. However, a few of those B cells remain in the system for years or even decades, and if a need arises, they can be stimulated to cause them to rapidly reproduce again, so that they and their progeny cells can rapidly begin making a renewed supply of the antibodies that were effective in helping fight off a particular type of microbe. Accordingly, if the same antigens that were introduced earlier (by a vaccine) are seen again, during a genuine microbial invasion, the “memory” B cells that were generated as part of the response to the vaccine will be (in effect) “scanned”, retrieved from a “library” or “archive” of candidate antibody-producing cells, and signaled to begin multiplying rapidly. Those B cells will begin secreting large numbers of antibodies, which will attach to the antigens on the invading microbe, acting as “flags” to attract and activate phagocytic cells, which will engulf and destroy the invading microbes. This process, as described above, mainly relates to Th2 responses.

By contrast, in a Th1-related response to a vaccine, antigen-specific memory CD8(+) T cells are generated. Like the antibody secreting memory B cells generated by vaccination, these memory CD8(+) T cells are primed to multiply rapidly, if the same antigen subsequently appears in the body. CD8(+) T cells are also known as “killer” T cells, because one of their primary roles is to detect and destroy cells that have become infected by invading viruses. By responding rapidly, in ways that do not require antibodies but that can kill a virus-infected cell before a virus has had enough time to fully take over a cell's internal machinery and make more viruses, antigen-specific memory CD8(+) T cells can recognize the early signs that a cell has been infected by a virus, and can destroy virus-infected cells, to minimize the number of additional viruses being made by the infected cells. The importance of Th1 responses and antigen-specific CD(+) T cells, in protecting against viral infections, is described in reviews such as Wiley et al 2001, and Wong and Pamer 2003.

Since a combination of both types of responses (Th1 and Th2) can be highly useful, in some situations, it would be desirable in such cases to administer a vaccine mixture that contains both (1) a first set of phage particles that will stimulate and drive Th1 responses, and (2) a second set of NALT-targeting phages that will stimulate and drive Th2 responses. Using influenza viruses as an example, NALT-targeting phages carrying TRL-related or other PAMP-related components, selected and designed to trigger a Th2 response, can carry one or more genes that encode the haemagglutinin (HA) and/or neuraminidase (NA) proteins. Those two proteins are exposed on the surfaces of influenza viruses, and they help flu viruses bind to and infect cells; as a result, those are the influenza proteins that can be readily bound by antibodies that are created by “memory” B cells. In the same vaccine mixture, NALT-targeting phages carrying TRL-related or other PAMP-related components that will trigger a Th1 response can carry one or more genes that encode the influenza proteins that take control over a host's cellular machinery, because those proteins are more likely to be present and active, in host cells that have been infected by the viruses and that need to be destroyed by killer T cells.

In addition to creating a “two-handed” vaccine mixture (which can also be referred to as double-barreled, double-pronged, or similar terms) that can be highly effective in responding to a particular microbe, another advantage of that type of approach should also be noted. By creating a vaccine mixture that can generate both (i) viral-antigen-specific memory B cells via a Th2 response, and (2) viral-antigen-specific memory CD8(+) T cells via a Th1 response, the vaccine can reduce the risk and probability that a single recombination event or mutational shift, in a pathogenic virus, would lead to emergence of a new and virulent strain that cannot be recognized by a vaccinated population, and which therefore might spread more rapidly and cause greater illness, suffering, and deaths.

### Other Potential Enhancements

After experts who works in this field recognize how the phage vaccine cassettes as disclosed herein can be constructed and used, potential enhancements will begin to be recognized by such experts, especially when additional articles have been taken into account. The identification and creation of such enhancements, after the basic logic, methodology, and structure of this new approach to vaccines is learned and understood, can be referred to by phrases such as “putting meat on the bones”.

As a demonstration, during a period of a few weeks after a meeting between the inventor and the patent attorney who drafted this application, while a draft of the initial provisional application was being prepared by the patent attorney, the inventor, with little or no assistance, identified the articles mentioned in this subsection, through a literature search. Each article cited in this section appears to offer or suggest what may become an important enhancement in one or more versions or embodiments of phage cassette vaccines as described herein. After experts who specialize in immunology have recognized how these types of phage cassette vaccines can be created and optimized, they will recognize various other, additional enhancements based on articles such as cited below. Accordingly, this list merely provides a starting list of potential improvements that merit attention, evaluation, and testing.

1. A CpG motif that may be effective for both avian and human vaccines has been identified and disclosed. Work with macrophage cells from poultry has shown that a B/K type CpG motif designated as “ODN 2006”, which is known to have strong stimulatory activity in human cells, also strongly stimulates costimulatory molecule expression in avian macrophage cells (Xie et al 2003).

2. It has been reported that certain DNA sequence motifs apparently can help stimulate the transformation and maturation of monocytes and/or macrophages into dendritic cells. Gursel et al 2002 reported that D-type ODN sequences could trigger human monocytes to mature into functional dendritic cells, while K-type ODN sequences did not have the same effect; the reported data also suggested that the cell maturation effects of the D-type ODN sequences apparently did not work via TLR9 mechanisms. In addition, Coban et al 2005 reported that when certain CpG motifs were inserted into plasmid DNA that was used for vaccination, PBMC monocyte cells could be stimulated to develop into mature dendritic cells.

3. In some situations, the genomes of opportunistic pathogens have evolved in ways that help those pathogens “fly under the radar” of a host's immune system. This usually happens when a mutant strain of a pathogen stumbles across a way to delete or at least hide, disguise, or obscure a certain component (such as a pathogen-associated molecular pattern, or PAMP, as described in the Background section) that the pathogen's hosts use as a way to recognize and identify that pathogen. In a situation of that nature, it may be possible to mark and even spotlight that type of pathogen, leading to efficient recognition and destruction by vaccinated hosts, by providing a vaccine component that provides the component of the pathogen that is missing or obscured.

As an illustration, since DNA sequences with CpG motifs in a pathogen's genome can stimulate active immune responses that will help a vertebrate host fight off invading pathogens, there has been selection pressure, on viruses that infect vertebrate animals, to reduce the relative frequency of CpG dinucleotides in their genomes. This has been described in articles such as Karlin et al 1994 and Kreig et al 1998, using examples such as influenza viruses with reduced CpG motifs, as indicated in Table 1 in Karlin et al 1994. Therefore, as reported in Cooper et al 2004, if conventional flu vaccines are mixed with oligonucleotide preparations that contain large numbers of CpG motifs, the oligonucleotides will act as adjuvants. The CpG adjuvants call attention to the presence of the injected vaccine particles, thereby boosting the immune system and pushing it into high gear. The activated immune system will then create an amplified response to the antigenic influenza protein sequences carried by the flu vaccines.

Accordingly, if a certain type of “stealth pathogen” has evolved in a way that avoids, minimizes, or delays detection by host animals, by a mechanism such as reducing the concentration, prominence, or other traits of a molecular pattern that hosts were using to identify the pathogen, then that “stealth pathogen” may be highly susceptible to a vaccine that specifically includes and utilizes the same type of signaling agent that the “stealth pathogen” managed to delete, minimize, or obscure.

4. It should also be noted that a similar concept can be made to work in reverse, in a very useful manner, when working with viruses that normally infect only bacteria and not mammals. In particular, the dinucleotide content of filamentous bacteriophages such as phage M13 evolved in ways that resemble and mimic the dinucleotide content of E. coli cells, which are natural hosts for the bacteriophages. That type of phage evolution allowed the phages to efficiently use the genetic machinery of their E. coli hosts (Blaisdell et al 1996). However, certain components of E. coli genomes (including large numbers of CpG motifs in their DNA) stimulate immune responses among mammals and other vertebrate animals. Therefore, since the genomes of phages that infect E. coli tend to have similarities and homologies with E. coli genomes, such phages come already equipped with CpG motifs, in their genomes, that will activate the same types of vertebrate immune responses activated by E. coli infections. That is a useful and fortunate factor, which will help improve the efficacy of phage cassette vaccines for use in vertebrate animals (e.g., Frenkel et al 2004).

5. Phage genomes can be manipulated, in ways that can allow the phage vectors to provide an array and assortment of different CpG motifs. Studies have shown that the response of monocyte cells, from different human donors, to stimulation by DNA sequences having CpG motifs, is not always consistent; no single oligo-deoxy-nucleotide sequence is maximally stimulatory in all human monocytes (e.g., Klinman and Currie 2003, and Leifer et al 2003). Therefore, a mixture of ODN sequences having different sequences and activities can be woven into a phage genome. This can be done, for example, by exploiting the redundancy of the genetic code, which allows any of several different codons to be used to specify a number of amino acids, with no change in the amino acid sequence of a resulting protein. The resulting phage cassettes may be able to induce the desired immune system activation in the widest possible range of recipients, in mixed populations that will have diverse genomes and potentially differing responses to such vaccines.

It also should be noted that research into DNA vaccines, which today mainly uses relatively short oligo-deoxy-nucleotide sequences that must be chemically synthesized, to avoid major and costly purification problems, has been stunted and limited by the costs of synthesizing the DNA strands used in such vaccines. By contrast, since phage cassette vaccines can be manufactured in huge quantities merely by culturing phage/bacteria mixtures with a few simple and inexpensive nutrients, the methods and approaches disclosed herein may well be able to push research into ODN's, DNA vaccines, and CpG motifs, to a much higher level of accelerated and fruitful research.

### NALT-Targeting Polypeptides in Eukaryotic Viruses

The third composition of matter disclosed herein comprises vaccines that contain targeting-and-delivery polypeptide sequences that were identified by screening of a phage display library, regardless of whether the vaccine particles are, or are not, phage particles. If a certain polypeptide sequence, carried by a phage particle that is one out of millions or billions of phages in a phage display library, has been discovered and shown to be highly potent and effective at triggering both (i) uptake into NALT cells, and (ii) transport into the phagosomes of macrophages, then that “target-and-deliver” polypeptide sequence can be used in ways that are not limited to vaccine particles made by phages.

For example, a target-and-deliver polypeptide sequence can be incorporated into various types of viruses that infect eukaryotic cells, rather than infecting bacteria. Such viruses are widely used in vaccines today, in either “disarmed” forms (which can also be referred to as attenuated, crippled, etc.) or in “killed” (or inactivated, nonviable, etc.) forms. Most “subunit” vaccines, which contain an antigenic polypeptide sequence from a pathogen that has been spliced into some other type of carrier-type virus, also use carrier viruses that infect eukaryotic cells, rather than bacteria.

These types of vaccines are reproduced in various types of eukaryotic cells, such as in bird eggs, insects or caterpillars (or insect cells grown in tissue culture), or human or monkey cells grown in cell culture (such as “Vero” cells, a cell line obtained from vervet monkeys in the early 1960's, widely used for making vaccines against polio and certain other diseases). All of those eukaryotic cell types can “glycosylate” vaccine particles, which refers to attaching sugar moieties to protein surfaces. Many pathogens have sugar moieties on their surface proteins, since a “cloud of sugar” can help obscure the nature of a pathogenic invasion, and give a pathogen a better chance to establish itself and reproduce before a victim's immune system can fully respond. Therefore, glycosylated vaccines can more closely resemble and mimic the surfaces of a pathogenic microbe that a vaccine is designed to defend against. Since bacterial cells normally cannot glycosylate proteins, glycosylated vaccines usually must be manufactured in eukaryotic cells.

Similarly, bacteria cannot perform some types of protein folding (i.e., shaping of a protein strand into a certain three-dimensional shape and conformation), or other types of “post-translational processing” performed by eukaryotic cells.

However, if a vaccine must be cultured and reproduced in eukaryotic cells rather than bacteria, this can impede the use of bacteriophage particles as vaccines, since bacteriophages normally infect only certain types of bacteria, and cannot infect animal cells.

Using genetic engineering methods, it often is possible to find ways around such obstacles. For example, eukaryotic cells can be genetically manipulated, to give them a foreign gene that will express a new surface protein that will serve as a binding site (or docking site, or similar terms) for phages. This can allow phages to enter eukaryotic cells which carry those proteins on their surfaces.

Alternately, after a “target-and-deliver” polypeptide sequence has been identified by screening a phage display library, that polypeptide sequence can be incorporated into a coat protein (or other surface protein) of other types of engineered viruses that are used to create vaccines. This approach can be used to create nasally-administered vaccines, using engineered viruses that can be cultured and manufactured in eukaryotic host cells (such as bird eggs, insects or caterpillars, yeast, mammalian cells, etc.) that will perform glycosylation, protein folding, post-translational, or other processing steps that may be necessary to give the vaccine a desired activity and potency. When administered via nasal spray (which can be a liquid, aerosol, powder, etc.), the “target-and-deliver” polypeptide sequence (which initially was discovered in a phage display library, and which later was “transplanted” into a virus that infects eukaryotic cells) will cause the viral vaccine particles to more readily bind to (and enter) NALT cells in inoculated animals, which will then deliver the engineered viral vaccines to macrophages, and possibly to one or more other types of phagocytic antigen-presenting cells. Accordingly, a “target-and-deliver” polypeptide sequence, when inserted into a type of vaccine virus that can be manufactured (and glycosylated or otherwise processed) in eukaryotic cells, can increase the potency and efficacy of the resulting vaccine, and can render the vaccine well-suited for administration using a nasal spray, rather than a needle.

Accordingly, NALT-targeting polypeptide sequences as disclosed herein can be inserted into vaccines such as influenza vaccines, which are manufactured by culturing viruses in eukaryotic cells, such as bird eggs. Flu viruses are among the most rapidly-mutating viruses known, and new mutants appear each year that can cause severe illness and major epidemics, even among people who have been exposed multiple times to previous flu infections and/or vaccines. Two major surface proteins of influenza are haemagglutinin (HA) and neuraminidase (NA). Therefore, each year, newly-updated vaccine strains must be generated, by using genetic engineering to alter a “master strain” that is attenuated (i.e., effectively crippled, so that it cannot cause major problems or severe illness). Using genetic splicing methods, partial amino acid sequences from the haemagglutinin and neuraminidase surface proteins of recently emerged wild-type strains that threaten large-scale epidemics are inserted into the attenuated “master strain”. This creates an attenuated vaccine carrying partial haemagglutinin and neuraminidase sequences that are carried by the most threatening wild-type mutant strains.

The teachings herein can be adapted for use within that system, by including (in the final versions of the influenza vaccine viruses) a “target-and-deliver” polypeptide sequence (such as the sequence disclosed herein) that will trigger and promote the two immune cell reactions discussed herein, which are: (i) uptake of the vaccine particles into NALT cells in the nasal and/or throat region, followed by (ii) transport of the vaccine particles into the phagosomes of macrophage cells, which will become antigen-presenting cells.

The preferred insertion site, for such a NALT-targeting polypeptide sequence, can be determined by experts working for a company that manufactures such a vaccine. Several companies manufacture such vaccines, and each vaccine is somewhat different; as just one example, an annually-updated, nasally-administered flu vaccine is sold by MedImmune, under the trademark NASALMIST.

The experts at any company that manufactures a flu vaccine know the entire DNA sequence of the genome of the particular “master strain” virus they use as their carrier. Based on that genome, they also know the complete amino acid sequences of all surface proteins on their “master strain”. From that starting point, it is within the skill in the art to select an insertion site, somewhere in the genome of the “master strain”, that will accommodate a relatively short segment of DNA (roughly 50 base pairs), in a position that will cause the altered gene to express a viral surface protein having a “target-and-deliver” polypeptide sequence as disclosed herein, in an exposed surface location. In most cases, the “target-and-deliver” polypeptide sequence (which typically will comprise a stretch of roughly 15 amino acid residues or less) can be used to replace a sequence having a similar length, or added as an “epitope tag”, presumably near the N-terminus or C-terminus as appropriate, in a surface protein of the attenuated master strain, so that the size of the modified surface protein will be unaltered, or altered only slightly. This can avoid creating surface proteins with substantially altered sizes, which might hinder assembly of the modified virus particles.

It also should be noted that in any viruses of interest for vaccine use, dozens or hundreds of copies of each surface protein are present, on the surface of each viral particle. This allows an approach in which two different genes can encode two different variants of a single surface protein. One of the two gene variants can be engineered to create a polypeptide with a NALT-targeting polypeptide sequence, as disclosed herein. That NALT-targeting surface protein (in multiple copies on each virus particle) will trigger entry of the vaccine particles into NALT cells, and then into phagosomes in macrophage cells. The other gene variant can be used to provide copies of: (i) an unmodified surface protein, if desired; or, (ii) a modified protein carrying an antigenic polypeptide sequence from a pathogenic microbe, which has been selected because it will trigger the formation of antibodies that will enable an inoculated host to mount a rapid immune response against the pathogenic microbe.

Other examples of viruses that can infect the upper respiratory system include coronaviruses (which includes SARS-CoV, a variant that causes “severe acute respiratory syndrome”, or SARS), picornaviruses, rhinoviruses, adenoviruses, etc. Most of these viruses (other than SARS-CoV) do not cause potentially lethal illnesses, so most of them have not received major attention in terms of vaccine development (a notable exception involves adenoviruses, which have been extensively studied and developed for various types of gene therapy, largely because they can be used with a class of so-called “helper viruses” (also called satellite viruses) that enable various useful techniques and safeguards). However, all of the virus types listed above can trigger immune responses. Accordingly, any of these types of viruses can be converted into nasally-administered attenuated carriers, which can carry exposed surface proteins that will include: (i) at least one NALT-targeting polypeptide sequence, which will efficiently deliver vaccine particles to antigen-presenting immune cells; and, (ii) at least one antigenic polypeptide sequence from a pathogenic microbe, which will be incorporated into the vaccine in order to trigger the formation of antibodies that will bind to the pathogen.

Another important class of viruses that can be improved by inclusion of NALT-targeting polypeptide sequences are called “nonsegmented negative-strand viruses” (NNSV). The term “nonsegmented” means that the entire viral genome is carried in a single molecule, of either single-stranded or double-stranded DNA or RNA (by contrast, some viruses require two different segments of DNA or RNA to be gathered and packaged into each viral particle). The term “negative-strand” (also called anti-sense strand, or nonsense strand) means that when a “complementary” copy of the viral DNA or RNA is formed by a cell, the “complementary” strand will be the “sense” strand, with codons that directly encode a viral protein. In addition, many NNSV viruses are surrounded by lipid membranes, usually called “envelopes”. These usually are obtained from a host cell by means of a “budding” process, in which each virus particle surrounds itself by an initial pocket that enlarges into a bubble-type enclosure, made from one or more membranes of a host cell.

All of those factors enable NNSV viruses to reproduce more rapidly than most other types of viruses that infect mammals. First, a requirement for only a single molecule of DNA or RNA, when virus particles are being assembled, leads to greater speed and reliability, compared to viruses that must assemble and package two different strands of DNA or RNA. Second, if a virus carries the “negative-strand” of a DNA or RNA molecule, then as soon as it “hijacks” the cell machinery and directs it to make more strands of RNA, the newly-formed “positive strand” RNA will have codon sequences that will cause the cells to rapidly make more viral proteins. Third, the ability of a virus to simply take part of a cell's membrane, rather than needing to make a complete set of coat proteins, reduces the number of molecules that must be created and assembled, to make more viruses; in addition, viruses that are released by a “membrane budding” process usually spare the lives of infected cells, allowing infected cells to make even more copies of the virus.

Due to those factors, NNSV viruses tend to pose the most rapid, acute, and aggressive pathogenic threats to health. They include, for example, rabies virus, measles and mumps viruses, sendai virus, human parainfluenza viruses, vesicular stomatitis virus, Newcastle disease virus, human respiratory syncytical and metapneumovirus, Ebola and Marburg viruses, and Bora disease virus. NNSV viruses are regarded as posing the greatest threats of bioterrorism, and they have received a great deal of attention and research.

The design and use of NNSV viruses, in vaccines, is reviewed in articles such as Bukreyev et al 2006. Since some types of NNSV viruses can efficiently infect nasal and throat tissues, those NNSV viruses offer promising vectors for creating attenuated or inactivated vaccine vectors, which can be enhanced by inserting NALT-targeting sequences into one or more of their exposed surface proteins. Such NALT-targeting sequences can accelerate and increase the entry of the resulting modified vaccines into NALT cells, and then into the phagosomes of macrophage cells.

It should be noted that NNSV vaccines that have been enhanced in that manner are not limited to vaccines for preventing NNSV diseases. Instead, NNSV vaccine vectors can be modified to include antigenic proteins that will immunize recipients against completely different types of pathogens.

In addition, the potency of at least some vaccines created from NNSV vectors (or from coronavirus, picornavirus, rhinovirus, or other viruses) is likely to be increased even more, by also incorporating one or more additional components that will activate one or more types of TLR receptors, or that can otherwise help direct and guide an immune response toward a desired response, such as either an MHC-1 or MHC-2 response, or a TH-1 or TH-2 response.

### Vaccines Against Cellular Pathogens

NALT-targeting polypeptide sequences as disclosed herein also can be incorporated into vaccines made from bacteria or other cellular (rather than viral) microbes. The term “cellular” is used conventionally, to exclude viruses while including bacteria, fungi (including yeast cells), mycobacteria, and other microbes that are regarded as “cells” by biologists. In general, viruses do not carry the enzymes necessary to metabolize nutrients, synthesize DNA or proteins, or make new lipid membranes, so viruses must obtain those building blocks from host cells. By contrast, with a few minor exceptions (mainly involving “auxotrophic” deficiencies in microbes that grow only in environments that supply any missing nutrients), cellular microbes carry the enzymes necessary to metabolize nutrients, synthesize DNA and proteins, and make lipid membranes.

Because various respiratory diseases involve mucosal tissues in the nose and throat, respiratory diseases are likely to be of early interest among researchers studying the disclosures herein. Examples of respiratory diseases caused by cellular microbes include tuberculosis (caused by Mycobacterium tuberculosis), pneumonia (caused by Streptococcus pneumoniae, also referred to as pneumococcus), and a disease of horses known as “strangles” (caused by Streptococcus equi). Vaccines containing killed or attenuated microbes are available for all three diseases, as described in various articles, government reports, and websites maintained by vaccine manufacturers. However, none of those vaccines are fully optimal, and improved vaccines for any of those diseases could be useful and helpful.

The same principle applies to vaccines for other diseases caused by cellular pathogens, regardless of whether they infect nasal or throat tissues. No vaccines are perfect, and the disclosures herein can enable improvements in numerous types of vaccines.

It also should be noted that two major diseases, malaria and AIDS, have frustrated all efforts to develop truly effective vaccines. Accordingly, it is not asserted or claimed herein that vaccines developed by the methods disclosed herein can provide fully optimal and ideal vaccines for all diseases. Instead, it is asserted that these methods and components will enable researchers and companies to create substantially improved vaccines for at least some diseases and pathogens. Whether this new approach can help create effective vaccines against the two most intractable challenges, malaria and AIDS, cannot be predicted as this application is being drafted and filed.

Any of several approaches can be used to incorporate NALT-targeting polypeptide sequences into vaccines against cellular (non-viral) diseases and pathogens. Briefly, three major routes include: (1) development of phage particle vaccines, which will include NALT-targeting polypeptide sequences identified by screening methods as described herein, combined with antigenic sequences derived from cellular (non-viral) pathogens; (2) development of viral vaccines derived from viruses (presumably glycosylated) that normally infect eukaryotic cells, which will include NALT-targeting polypeptide sequences, combined with antigenic sequences derived from cellular (non-viral) pathogens; and, (3) development of killed or attenuated cellular vaccines, which will incorporate NALT-targeting polypeptide sequences, and antigenic sequences derived from cellular (non-viral) pathogens, into various cell-surface proteins.

Preferably, when researchers attempt to determine the optimal type of vaccine against a particular disease caused by a cellular (non-viral) pathogen, all three approaches mentioned above should be tried and tested, to prepare different classes of candidate vaccines. Each such candidate vaccine class can be tested in animals, to determine which class appears to work best, for that particular disease. Candidate vaccines which show the most promise in animals can then be tested in humans. Such tests normally require one or more rounds of non-pathogen tests, in which no infections, exposures, or “challenges” by pathogens are involved. These tests are used to evaluate a candidate vaccine's safety, any unwanted tendencies to provoke allergic, tolerance or other undesired reactions, and its ability to trigger the formation of circulating antibodies that will actively bind to the pathogen, in in vitro tests. If a candidate vaccine performs well in the safety tests, it can be tested for efficacy against actual pathogens, using known procedures (such as inoculating a large population of people who are at elevated risk of a certain disease, then gathering statistical data on how many inoculated people contracted the disease, compared to how many people contracted the disease in an untreated population of the same size).

### Vaccines for Fighting Cancer and Similar Disorders

As mentioned at several locations above, various types of vaccines are being tested and used in the hope that they will be able to help patients fight and overcome various nonmicrobial diseases. Cancer vaccines have been extensively researched, as described in sources such as Acres et al 2007 and www.cancer.gov/cancertopics/factsheet/cancervaccine. While some types of cancer can be caused by viral infections (such as cervical cancer, caused by papilloma viruses), most cancers have no specific microbial cause, and arise from mutations that can occur when cells reproduce. In addition, even if a cancer is caused by a virus or other microbe, the nature of the disease requires that the cancerous cells must be attacked and destroyed, and the initial causative factor has little or no importance after cancerous cells begin replicating uncontrollably.

Other nonmicrobial disorders that may someday benefit from vaccine therapy include Alzheimer's disease, autoimmune disorders, hormonal or endocrine disorders, and other disorders that arise when native cells, body parts, or metabolites go wrong, rather than arising from infections by microbes. Information on how vaccines might benefit such nonmicrobial disorders can be located easily by searching the U.S. National Library of Medicine database, or various Internet databases.

When vaccines for noninfective disorders are involved, the distinctions between MHC1 and MHC2 molecules, and between Th1 and Th2 immune responses, will need and merit careful attention, because “cell-mediated” Th1 responses (rather than Th2, humeral, IgG-antibody responses) likely will offer the best routes for treatment. For vaccines that are intended to target cancer cells or other native cells (rather than targeting microbial invaders), MHC1 receptors and TH1 cells may be usefully involved, and will deserve close attention.

It is believed that the methods and reagents of this invention will enable phage vaccines to be developed that will be able to specifically target either MHC1- and TH1-mediated cells, receptors, and/or processes, or MHC2- and TH2-mediated cells, receptors, and/or processes. This will enable the extension of the types of phage vaccines described herein, into both: (i) potent and efficient treatments for cancer and other diseases; and, (ii) double-pronged approaches that can provided improved defenses against some types of microbial diseases.

In general, approaches that can extend NALT-targeting vaccines (as disclosed herein) into uses for treating cancer or other nonmicrobial diseases can be summarized as follows:

(1) large numbers of phage particles, having a variety of different polypeptide sequences in their coat proteins, can be nasally administered, in the form of a phage display library suspended in a liquid or powder solution;

(2) the NALT cells in the treated animals will take in, transport, and process some of those phages, in certain ways;

(3) those phages that were selected and transported by an animal's NALT cells can be isolated and reproduced;

(4) the isolated NALT-targeting phages can be tested, using cell culture methods, to determine whether they activated either: (i) MHC1 receptors and TH1 cells, or (ii) MHC2 receptors and TH2 cells. This can be done, for example, by using assays that can detect interleukin-2 or gamma-interferon proteins (which will indicate that TH1 cells were stimulated), versus assays that can detect interleukin-4, interleukin-5 or interleukin-10 proteins (which will indicate that TH2 cells were stimulated).

The foreign DNA and polypeptide sequences carried by NALT-targeting phages that activate MHC1 receptors and TH1 cells can then be used as targeting polypeptides, in vaccine preparations that use clonal phage particles that are ideally sized and suited for such use. These types of anticancer (or similar) vaccines will be used to deliver selected antigen protein sequences to T cell types that, when activated, will begin killing and destroying cancer or other disease-causing cells.

As mentioned above, those types of cell-mediated immune responses, by activated T cells responding to antigenic polypeptides that were presented to them by macrophage cells, can be triggered by a sequence of steps such as the following:

(1) a gene that encodes a known antigenic polypeptide sequence, which will have the same amino acid sequence as a cancer-related antigen found in large numbers on the surfaces of certain types of cancer cells, is inserted into a NALT-targeting phage cassette that activates MHC1 receptors and TH1 cells preferentially over MHC2 receptors and TH2 cells;

(2) the resulting anti-cancer vaccine, carrying both (i) a cancer-antigen protein sequence, and (ii) a NALT-targeting polypeptide, is administered to an animal or patient, via a mucosal mode, such as a nasal spray;

(3) the phage vaccine particles will be transported into NALT tissue, and it then will be taken into the phagosomes of macrophage cells, because of the target-and-deliver protein sequence inserted into its coat proteins;

(4) at least some of the macrophages will convert into antigen-presenting cells, which will travel to lymph nodes and “present” the phage antigens to T cells;

(5) the phage components that were selected and used, in that particular type of vaccine cassette, because they activate MHC1 receptors and TH1 cells, will do their work, and will steer and guide the immune system into launching a cell-mediated immune response, which uses activated T cells, rather than triggering formation of IgG antibodies via B cells;

(6) at least some of the activated T cells will bind to cancer cells that have, on their cell surfaces, the same cancer-related antigenic protein sequence that was placed into the coat proteins of the genetically-engineered anti-cancer vaccine particles;

(7) after an activated T cell attaches itself to a cancer cell that contains the same antigenic protein sequence that was present in the cancer-fighting vaccine, the activated T cell will inject perforin into the cancer cell;

(8) the cancer cell's mitochondrial membranes will become permeable, due to the action of the perforin from the activated T cells; and,

(9) the induced mitochondrial permeability, inside the cancer cell, will lead to mitochondrial release of a signaling molecule called “cytochrome c”, which will trigger a series of events leading to the apoptotic death and destruction of the cancer cell.

In summary, by using an appropriate screening test on millions or billions of candidate phages in a phage display library, NALT-targeting polypeptides can be screened and selected. Particular polypeptide sequences, selected because they performed efficiently in those screening tests, can then be incorporated into the coat proteins of phage vaccines (or other types of vaccine particles, as described above), by genetic engineering methods. This will create “cassette”-type phages, which can then be modified by a final step to turn them into cancer-fighting (or similar) vaccines. These “cassette”-type phage vaccine vehicles can be supplemented, by inserting into the phage genome an additional gene sequence, which will encode (in one of the phage coat proteins) a second foreign polypeptide sequence, which will be a known cancer antigen. The resulting anti-cancer phage vaccine particles will have: (i) a targeting sequence that makes the phage particles efficient in promoting uptake by NALT cells, followed by uptake into the phagosomes of macrophages; (ii) one or more components that will steer and guide the response by the macrophage cells in a manner that activates MHC1 receptors and TH1 cells preferentially, over MHC2 receptors and TH2 cells; and, (iii) an antigen sequences that will cause activated T cells to seek out and destroy cancer cells that have the same antigen proteins on their surfaces.

### Vaccines Against Peptide Hormones

Another class of vaccines that can be enabled and/or enhanced by the disclosures herein involve vaccines that will trigger the production of antibodies that will bind to one or more types of peptide hormones, or hormone receptors.

In mammals and birds (and likely in many types of reptiles also), the central nervous system, the endocrine and paracrine systems, and certain other body parts use various types of hormones that are peptides (i.e., formed by linking amino acids together). A major reason for the use of peptide hormones is that their concentrations can be tightly and reliably controlled, through mechanisms that directly control the expression levels of genes. By contrast, nearly all nonpeptide (or “small molecule”) hormones (such as adrenalin, estrogen, and testosterone, as examples) are made by enzymes that will chemically modify any precursor (or “substrate”) molecules they encounter. Because nearly any such enzyme will convert any and all available precursor molecules into product molecules, it is much more difficult to reliably control the quantity of small-molecule hormones that are made by enzymes.

In a number of situations, it can be very useful to reduce or inhibit the activities of certain peptide hormones. As just one example from human medicine, two peptide hormones called “follicle-stimulating hormone” (FSH) and “luteinizing hormone” (LH) have been shown to accelerate and worsen brain damage and dementia, caused by Alzheimer's disease in elderly humans (e.g., U.S. Pat. No. 6,242,421, Bowen 2001). Therefore, if vaccines could be developed that would trigger the production of antibodies that would bind to (and thereby inactivate) FSH and/or LH (or GnRH, which stimulates the pituitary gland to secrete FSH and/or LH), such vaccines might be able to help treat and minimize Alzheimer's disease. Similar examples can be provided for various types of cancer that are “fueled” by certain types of peptide hormones (including prostate cancer, as one example).

In animal medicine, several examples merit brief attention. As one example, in livestock, a hormone called “gonadotropin” (Gn), which is released under the control of an “upstream” hormone called “gonadotropin release hormone” (GnRH), causes powerful effects in sexual maturation, and in the cycle of estrus. Therefore, vaccines have been developed that trigger the production of antibodies that bind to GnRH. These vaccines are used to control estrus and estrus-related behavior in mares, and as an alternative to castration of males (e.g., Thompson 2000; Naz et al 2005; Elhay et al 2007).

As another example, animal growth is moderated and controlled by a balance between two offsetting peptide hormones. The term “somatotropin” is a technical (and marketing) term for “growth hormone”; the root “somato-” refers to body, and “-trop” refers to nutrition, sustenance, and/or growth. The offsetting (growth-inhibiting) hormone is called “somatostatin”, where “statin” indicates “unchanging” (as in found in words such as stay, stable, and stationery). Therefore, if a vaccine could potently trigger the production of antibodies that bind to and inactivate somatostatin, animals raised for food (such as poultry, hogs, cattle, etc.) might be induced to grow larger, with more muscle and less fat, without requiring the injection of any growth hormones. Since injecting food animals with growth hormones raises concerns and objections among many consumers, health care experts, government agencies, and others, the potential for a simple one-time vaccine to produce larger, faster-growing, and more food-efficient livestock merits attention. Prior efforts in this field have been described in articles such as Xu 1994; however, no such vaccines have yet been developed that are optimal and effective.

Those are just brief examples of how vaccines that would trigger formation of antibodies that will bind to peptide hormones might be used, both in human medicine, and in agricultural and/or veterinary medicine. Other potential uses are known to endocrinologists and other specialists.

It also should be noted that vaccines are being developed and tested for birth and population control, in humans and in various animals. Such vaccines are reviewed in Naz et al 2005, and numerous other sources. For example, using the technology disclosed herein, painless nasal administration of a vaccine to dogs or cats may be able to eliminate the need for surgical neutering.

Another major implication of the hormone-related teachings herein should also be noted. Most mammalian hormones (either peptide or nonpeptide) act by binding to a receptor protein that is exposed and accessible on the surfaces of cells. In some situations, it may be useful to administer a vaccine that will trigger the production of antibodies that will bind to, occupy, and effectively block and inactivate certain types of hormone receptors, rather than binding to the hormones. In general, in young patients who have not yet reached or passed child-bearing age, those types of vaccines normally would be used only in dire circumstances, such as to combat lethal diseases. Among people who have passed beyond their child-bearing years, such vaccines might be useful in a broader range of therapies.

Screening of Phages that Enter Blood or Pass Through Membranes

The types of screening tests disclosed herein also can be used to identify particular phages, from a display library, that will pass through one or more types of biological membranes.

For example, during the early stages of this research, the inventor set out to isolate and identify polypeptide sequences that could be used to efficiently carry genetic vectors and other payloads from nasal cavity into the blood circulation. To accomplish that, phage display libraries were nasally administered to mice. At various times after intranasal administration, animals were anesthetised, and blood was sampled. The results indicated that phages appeared within the blood circulation within 15 minutes, and were rapidly cleared from the blood.

Phages in the blood were reproduced in E. coli, and the resulting phage populations were tested by additional rounds of nasal-to-blood in vivo selection. Increasing numbers of phages were recovered with each selection round, during several rounds of testing. When fully-diverse scFv phage were used, initial nasal-to-blood screening tests yielded highly variable results. When similar tests were performed using scFv phage that previously had been enriched by sciatic nerve screening (as mentioned above, and described in more detail in WO 2003/091387), larger and more consistent numbers of phages were obtained.

Accordingly, the phages selected by those types of screening tests were demonstrated to pass through endothelial membranes (i.e., the class of membranes that form capillaries and other blood vessels); and, the polypeptide sequences carried by those phages were shown to efficiently drive the transport of particles through endothelial membranes, into circulating blood.

However, even after multiple rounds of in vivo selection and enrichment, the number of scFv phage that could be recovered from the blood was only about 10 to 20-fold higher than observed with a control phage population. This indicated that a saturation phenomenon was occurring, which presumably involves a limited number of portals (such as M cells) through which phage were passing from the nasal cavity, into circulating blood. Despite repeated rounds of in vivo selection, the maximum load of phage particles that could be delivered into the blood was estimated to be less than 0.0001% of the administered dose. This posed an inherent limit on the usefulness of this route, for systemic delivery of drugs or vaccines, and it helped motivate the Inventor herein to redirect his efforts into developing a different type of nasal in vivo screening strategy, aimed not at isolating phages that could deliver payloads through M cells into circulating blood, but instead, at isolating phage-ligands that would be partially retained within the NALT, in a manner that could enable delivery of the phages to antigen-presenting cells of a mucosal immune system.

Since intravenous injection (and in some cases skin patches or other modes of administration) can introduce any pharmacological agent directly and efficiently into circulating blood, and since various approaches are known for directly controlling the sustained and prolonged release of drugs, it is not clear how that discovery would have substantial commercial or therapeutic value; furthermore, the “apparent saturation” factor mentioned above must also be taken into account. Nevertheless, those early tests are mentioned herein, partly for the sake of completeness, and partly because others may be able to identify and develop practical uses for that discovery.

The disclosures herein also indicate that screening tests, using phage display libraries as candidate starting populations, can be used to isolate and identify polypeptide sequences that specifically target GALT cells (i.e., gut-associated lymphoid tissues). Because of the importance of food-borne microbes throughout the course of evolution, the intestinal tract contains specialized clusters of “lymphoid follicles”, including structures called “Peyer's patches”, located mainly in the small intestines (as an indicator of their importance, it has been estimated that 70% of a mammalian immune system, by volume, resides in the digestive tract). As with NALT tissues in the nose and throat, GALT tissues in the gut are covered by “M cells” having specialized microfolds, which create expanded surface areas that enable the M cells to “sample” molecules passing through the intestines, and to transfer those molecules or particles that appear unusual (such as molecules with “pathogen-associated molecular patterns”, or PAMPs, described in the Background section) to immune cells that reside or travel beneath the outer layer of M cells.

In general, unless and until experimental data indicates otherwise, it is presumed herein that NALT-targeting vaccines are like to be more efficient than comparable orally-ingested GALT-targeting vaccines. GALT-targeting vaccines will need to be specially formulated to survive stomach acidity, presumably by placing them in carriers that will release the vaccine particles only after the vaccine reaches the small intestines (one such carrier, developed by students working with Prof. Hai-Quan Mao at Johns Hopkins University, has been announced for a rotavirus vaccine being developed by Aridis Pharamaceuticals, www.aridispharma.com). However, even in such cases, the vaccine particles will be mixed with relatively large quantities of food that is being digested and converted into feces, in the intestines, and that natural process will inevitably reduce the contact and uptake of the vaccine, compared to a nasally administered vaccine that can be: (i) emplaced directly on NALT surfaces, in the nasal sinuses and (ii) held in position for sustained times by a mucoadherent compound, as described in the Background section.

Nevertheless, several major research efforts are underway to try to develop genetically engineered plants (or microbial feed additives) that will create proteins that, when eaten by poultry, livestock, and possibly humans, will effectively serve as vaccines against certain diseases. Accordingly, NALT-targeting and/or GALT-targeting transport polypeptide sequences can be merged and combined with such efforts, to utilize the potent and specialized transport activities of such polypeptide sequences.

Because of the close similarities between, and the essentially identical functions of, NALT tissues in the nasal and throat regions, and GALT tissues in the intestines, it is likely that the same polypeptide sequences disclosed herein which drive NALT intake followed by APC intake and processing, will also drive GALT intake followed by APC intake and processing. Alternately or additionally, analogs (described below) of any NALT-targeting polypeptide sequence, having limited amino acid substitutions, can be evaluated for potent GALT uptake activity.

If other polypeptide sequences that will potently drive both GALT uptake and APC phagocytosis are desired, such sequences can be isolated and identified by using screening tests that are directly comparable to the NALT screening tests described herein, modified in ways that will involve direct contact with GALT cells, rather than NALT cells. In general, instead of administering a phage library (with millions or billions of candidate phages) via nasal spray, a phage library can be fed to lab animals, using a suitable carrier system (such as the carrier mentioned above, for a rotavirus vaccine that will be taken orally). After a suitable time, the animals will be sacrificed, and Peyer's patch tissues (or similar intestinal tissues, or possibly certain types of “downstream” tissues or cells, such as macrophages in lymph nodes that serve the digestive tract) will be harvested. The cell membranes will be lysed, using a buffer that does not damage the coat proteins of the phages (as described for the NALT screening tests), to harvest enriched populations of phages that were preferentially taken into the Peyer's patch or other GALT cells. If desired, a phage library can be labeled, using FITC or other labeling agents, to make the tracking and harvesting steps easier or more efficient.

That type of screening process can be repeated as many times as desired, and the resulting enriched phage populations can be screened again, using macrophage association and/or phagosomal entry screening tests, as described herein.

Accordingly, if desired (for example, if a NALT-targeting polypeptide sequence that functions in a certain class of animals does not also function optimally for GALT intake, in that same class of animals), a sequential combination of screening tests, as described above, can be used to identify one or more phages, from a phage display library, that will potently drive both steps of a two-step process: (i) intake into GALT cells in the intestines, followed by (ii) phagocytic intake, by antigen-presenting cells.

If desired, in vitro screening tests can also be used, in one or more assays designed to identify enrich any GALT-targeting polypeptide sequences. For example, O'Mahony et al 2004 describes the use of a tissue culture method for screening a phage display library, using “CACO” cells from a transformed cell line that was initially derived from a colon cancer tumor. CACO cells (which are anchorage-dependent) can be grown into a cohesive layer which will have an “apical” side (which normally would be exposed to semi-digested food passing through the intestines), and a “basal” side. O'Mahony et al contacted the apical sides of their CACO cell layers with phage populations, and selected phages which passed through those cell layers.

Those tests did not involve vaccine testing or development, and no one should rely on the presumption that transformed (cancerous) cell lines will behave identically to healthy cells or non-tumorous tissues. Nevertheless, that report can be consulted for information on how in vitro tests have been performed using cell lines derived from intestinal tissues.

### Polypeptide Analogs for Targeting-and-Delivery Use

Any DNA or polypeptide sequence disclosed herein, and any other DNA or polypeptide sequence hereafter discovered to have potent targeting-and-delivery activity for vaccine use, can be used as a starting sequence (which can also be called a baseline, initial, or reference sequence, or similar terms), in efforts to find analogs having comparable or even higher potency.

In conventional chemistry and pharmacology, an analog is a molecule that resembles a certain designated molecule (which can be called a baseline, starting, initial, reference, or referent molecule or compound, or similar terms), but which has been modified by substituting or altering one or more groups or substituents of the starting molecule. For example, if a molecule has a relatively small “moiety” (i.e., an atom or cluster of atoms, such as a hydrogen, sulfur, or halogen atom, or a hydroxyl, amine, methyl, or similar small group) at a specific location on the compound, analogs can be formed by replacing that moiety with various other atoms or small groups, or by moving a moiety to a different location on the molecule. Similarly, saturated bonds can be replaced by unsaturated bonds, and various other limited modifications can be made, to create molecules that are similar but not identical to a starting compound. Such analogs can then be screened, to determine whether any of them have a comparable (or improved) level of a desired activity, compared to the starting molecule. If a particular analog is discovered to offer a significant improvement, it can then be tested and studied more closely, and it also can be used as a new starting or baseline molecule, in subsequent efforts to develop even better analogs.

Most research labs which do this type of work use computerized machines with transport mechanisms that will sequentially deliver dozens or even hundreds of small containers (such as glass vials, wells in a multi-well plate, etc.) to one or more sophisticated detector devices. Each vial, well, or other container holds a separate sample (or “aliquot”) of some test compound, usually created by an assay of some sort, which enables a product formed by some reaction or series of reactions to be tested. The detector device will analyze each aliquot, in turn. In some cases, this is done by taking and processing a sample of liquid from each container; in other cases, it is done by nondestructive means, such as by shining a light having a certain wavelength (or range of wavelengths) through each container, to measure one or more factors such as turbidity or color intensity (or to create a graph showing various peaks, as a function of differing wavelengths). The results of each test will be recorded in a computer, with identifying numbers to correlate each analytical result with a specific container and aliquot. The computer software can even rank the output data, so that the best-performing analogs can be quickly identified and ranked.

These types of programs and machines, to create and test large numbers of analogs with minimal effort, expense, and delays, have become a standard part of biochemical research, and the types of automated machinery that enable “high-throughput” testing of large numbers of analog compounds are described in articles such as Muller et al, Catalysis Today 81: 337 (2003). In the specific field of polypeptide chemistry, analogs of a starting polypeptide are created, by altering one or more amino acid residues in the amino acid sequence of a starting (or initial, baseline, etc.) compound. Such alterations can take the form of: (i) substituting (or “swapping”) one or more specific amino acid residues, without altering the number of residues in a sequence; (ii) inserting one or more additional amino acid residues into a known sequence, in a way that increases the number of amino acid residues in a sequence; or, (iii) deleting one or more amino acid residues, to decrease the number of residues in the sequence. These types of alterations can be created by well-known methods, such as by using automated machinery to create synthetic segments of DNA, which can be spliced into a known restriction site in a gene that encodes a coat protein, in a plasmid and/or phage. The synthetic segments of DNA can have either: (i) exact and known sequences, in “controlled mutagenesis”; or (ii) random and assorted sequences, in “random mutagenesis”. The resulting modified genes can then be expressed into polypeptides (by using phage vectors, microbial fermentation, or other methods), and the resulting phages or polypeptides can be tested, using cell culture, small animals, etc., to determine which particular polypeptides happen to have the strongest levels of activity, using any screening test that is of interest.

Accordingly, this invention anticipates that analogs can be prepared and tested, using any DNA or polypeptide sequence that is disclosed herein (or that is hereafter discovered to function potently as a targeting-and-delivery sequence, when used as disclosed herein), as a starting sequence. If some particular analog sequence is found to be more potent for the purposes disclosed herein than the starting sequence it was derived from, then that analog may rise to the level of a patentable improvement; nevertheless, if it is created and tested as an analog of a known sequence, using processes such as controlled or random mutagenesis following by screening tests as disclosed herein, then any such analog that arises from the teachings herein is within the scope of this invention.

### EXAMPLES

### Example 1

### Supplies and Methods

Adult male BALBc mice were used for in vivo selection studies. All animal tests were approved by an Institutional Animal Welfare Committee, under Australian law.

A phage display library, believed to contain 1.3×10¹⁰ individual recombinants, each containing a single chain variable fragment (scFv) gene sequence derived from human B-cells, and a CANTAB6 control phage lacking an scFv insert, were obtained from Cambridge Antibody Technology (United Kingdom). These phages carry an ampicillin resistance gene, as well as a plasmid origin of replication.

In vivo selection of the scFv phage library, to select and isolate phages that were taken into and retrogradely transported by nerve fibers in the sciatic nerve bundle in rats, is described in PCT application WO 2003/091387. In the discussion herein, preselected populations of phages that were used during specific steps are described by phrases such as, for example, “sciatic (18 hr)|diverse scfv”, which refers to a line of phages that were isolated from rat sciatic nerve tissue which was harvested 18 hours after administration of a scFv phage library to the sciatic nerve at a different location.

A second type of “peptide display” phage library was obtained from George Smith (University of Missouri, USA). It is believed to contain approximately 10⁸ different clonal phages, and is derived from a filamentous phage designated as fd-tet, which carries a tetracycline resistance gene (as a selectable marker), and an origin of replication that allows the phage genome to be manipulated and reproduced in double-stranded DNA form, as a plasmid. These phages also are known as “type 88” phages (Smith 1993), since their genome (9273 bases) carries two different genes that encode coat protein VIII. One of the two protein VIII genes carried by “type 88” phages is a wild-type (unmodified) gene, while the other coat protein VIII gene carries a foreign gene sequence that encodes a “15-mer” polypeptide (i.e., an inserted polypeptide sequence containing 15 amino acid residues, spliced into the normal amino acid sequence of the phage's coat protein VIII). This vector is also referred to as a f88-15mer phage library (GenBank accession number AF246448). The random 15-mer polypeptide sequences typically are expressed at up to about 300 copies per phage.

When appropriate, f88-15mer phages that were selected by some particular screening round as described below were reproduced (“amplified”) in the K91Kan strain of E. coli, a lamba-derivative of a strain known as K-38. Unless transformed by a phage or plasmid carrying a resistance gene, K91 cells are susceptible to the antibiotics kanamycin (used at 50 micrograms/milliliter, ug/ml) or tetracycline (20 ug/ml). In addition, an inducible promoter was placed in front of the gene that encodes coat protein VIII; this allows a compound known as IPTG (1 mM) to be used, when desired, to increase expression levels of recombinant coat proteins containing the various 15-mer foreign inserts that have been inserted into the coat protein VIII coding sequence.

The 15-mer phage library, or the scFv phage library, were cultured and reproduced, respectively, by methods described by George Smith (e.g., Smith 1993), by Cambridge Antibody Technology (described in their guides to users), and by references such as Bonnycastle et al 2001.

For example, when a round of in vivo phage selection using the scFv phage library was completed by means of the sciatic nerve method, the harvested phages (which can be released from the neurons by mechanical or ultrasonic homogenization and/or dissolution of the nerve cell membranes using a detergent) were incubated for 1 hour with a TG-1 strain of E. coli, in their log growth phase, in 2TY cell culture medium. The phage-infected E. coli cells were grown overnight in shaker flasks, in 400 ml of 2TY medium containing 100 ug/ml ampicillin. Infected E. coli (carrying phages with ampicillin resistance genes) were pelleted by centrifugation at 3000 rpm for 20 min, then taken up in 10 ml of 2TY medium, and a 10-fold MOI of helper phage M13KO7 carrying a kanamycin resistance gene was added. After 60 min, doubly-infected E. coli were added to 800 ml 2TY medium containing 100 ug/ml ampicillin and 50 ug/ml kanamycin, and grown for 24 to 48 hours at 37 C in a shaker flask at 300 rpm to allow secretion of phage into the medium. The E. coli were then removed by centrifugation at 10,000 rpm for 30 min. 20% v/v of 16.7% polyethylene glycol (PEG) with 3.3 M NaCl (Bonnycastle et al 2001) was added to the supernatant, to precipitate the phage overnight at 4 C. The PEG-precipitated phage were pelleted at 10,000 rpm for 30 min, then dissolved in 40 ml of Dulbecco's phosphate buffered saline (PBS), pH 7.4, and centrifuged at 4000 rpm for 20 min to clear the phage solution of any undissolved phage or other particulate matter. PEG precipitation was repeated at least two more times, prior to aliquoting the phages into sterile screw-cap vials. Phage were stored as PEG precipitates at 4° C. until subsequent use. Phages from f88-15mer were produced using similar methods, using LB medium with 12.5 ug/ml tetracycline in place of ampicillin, and without using helper phage. Phage-infected host cells in PBS or 2TY medium were stored at −80° C. after mixing 1:1 with cell freezing medium, which contained 88 g/L glycerol, 12.6 g/L K₂HPO₄, 3.6 g/L KH₂PO₄, 1.8 g/L (NH₄)₂SO₄, 0.9 g/L Na₃citrate, and 0.18 g/L MgSO₄-7H₂O).

When a need arose to produce still larger quantities of phage, a culture of TG1 E. coli cells in 2TY medium was grown to logarithmic growth phase, then infected with scFv phages (either from the complete diverse library, or from a previously selected population). The phage-exposed cell population was inoculated into 800 ml of 2TY containing ampicillin, and grown overnight in a shaker flask. The next morning, phage-infected E. coli were pelleted by centrifugation at 3000 rpm for 20 min, and the cell pellet was taken up in 10 ml of 2TY medium. M13KO7 helper phages were added, and allowed to infect the cells for 1 hour. To produce phage, infected host cells were grown in a 2 L shaker flask containing ampicillin and kanamycin supplementing 800 ml of medium with a recipe based on Liu et al 2000; in addition to 40 g/L D-sorbitol as a carbon source, this medium contained 5 g/L yeast extract, 5 g/L tryptone, 7 g/L NaH₂PO₄-12H₂0, 4 g/L KH₂PO₄, 4 g/L K₂HPO₄, 1.2 g/L (NH₄)₂SO₄, 0.2 g/L NH₄Cl, 2.4 g/L MgSO₄-7H₂O, and 0.02 g/L CaCl₂.

### Example 2

### Purification of Phage Using Ceramic Hydroxyapatite

Affinity column purification of phages was carried out using particulate hydroxyapatite (HA), a ceramic material containing calcium and phosphate, purchased from BioRad. Initial studies, using buffer recipes described in Smith and Gingrich 2005 for use with other types of hydroxyapatite affinity media, failed to achieve desired purification when particulate HA was used with the phages, so a trial-and-error series of tests were performed to adapt the buffers and methods of Smith and Gingrich 2005 for use with the HA-phage combination of the inventor. Briefly, a 4.5 ml HA column was prepared using 2.5 gm HA. 5 ml of a phage preparation, at a density of about 3 mg/ml in 20 mM maleic acid (pH 5.5) containing 2 mM CaCl₂, was loaded onto the column under gravity percolation. Subsequent elution buffers were prepared using 400 mM NaH₂PO₄.2H₂O, adjusted to pH 7.0 using NaOH.

A series of elution buffers was then passed through the loaded column, using 1 ml aliquots of elution buffer, and collecting 1 ml liquid fractions that emerged from the column for analysis, using elution sequences such as listed below, which provided good results:

Fractions 1-10: 20 mM maleic acid, pH 5.5, plus 2 mM CaCl₂

Fractions 11-20: 20 mM MOPS, pH 6.5

Fractions 21-30: 100 mM NaH₂PO₄, adjusted to pH 7.0

Fractions 31-40: 100 mM NaH₂PO₄, pH 7.0 plus 2.55 M NaCl

Fractions 41-50: 100 mM NaH₂PO₄, pH 7.0

Fractions 51-70: 250 mM NaH₂PO₄, pH 7.0

Fractions 71-80: 1M NaH₂PO₄+150 mM NaCl

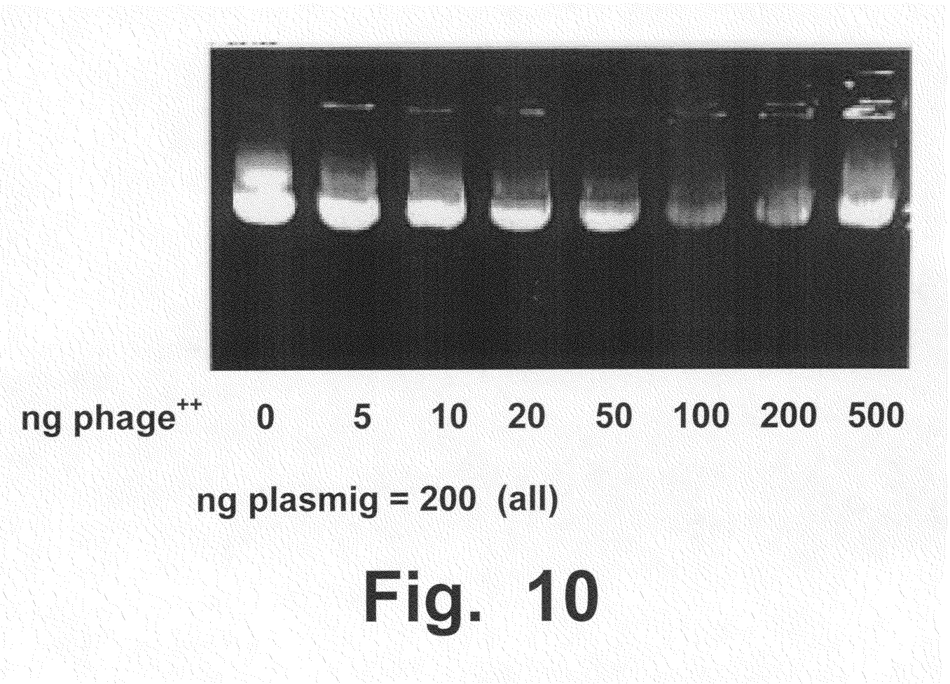

Protein content of the eluted fractions were optically monitored at a light wavelength of 280 nanometers (nm), in part because maleic acid buffer absorbs light strongly, at wavelengths below 280 nm. The elution profiles were generally similar to those reported in Smith and Gingrich 2005, and highly purified phages eluted in fractions 60 through 70, as illustrated in FIG. 10.

### Example 3

### Use of Anti-Phage Antibodies

To prepare antibodies that would bind to the phages, two sheep were immunized with phage. After a suitable delay, blood was sampled, serum was isolated, and the serum (containing IgG antibodies) was precipitated with 50% ammonium sulfate. The antibodies were resuspended and passed through an affinity column containing immobilized phages.

To immobilize phages in the affinity column, ethylene dichloride (EDC) was used for crosslinking 10 mg EDC was added to 1 ml 40 mM NaH₂PO₄ (adjusted to pH 7.0 by NaOH) containing 16 mg purified CANTAB6 phages. The mixture was allowed to react for 4 hours at room temperature, then another 10 mg of EDC was added, and the mixture was incubated (with mixing) for another 4 hours. The EDC-crosslinked phage were precipitated overnight using polyethylene glycol, then dissolved in 5 ml of coupling buffer (0.1 M NaHCO₃, pH 8.3, plus 0.5 M NaCl). In parallel, 1 gm of CNBr Sepharose 4B was washed with 1 mM HCl, then added to the coupling buffer containing EDC-crosslinked phages. The mixtures was incubated for 4 hours, then quenched with 0.1M Tris (pH 7.6) and incubated overnight. Unbound phage and coat protein components were removed, using 4M MgCl₂+50 mM acetate, pH 5.0.

When the blood serum fraction (enriched in IgG by ammonium sulfate precipitation, and processed to remove red and white blood cells from the phage-injected sheep) was passed through the affinity column, antibodies with phage affinity remained bound to the immobilized phages in the column, while other molecules were washed out of the column. A strong elution buffer (4M MgCl₂ plus 50 mM acetate, ph 5.0) was then passed through the column, to release and remove the phage-bound antibodies.

To prepare ELISA (enzyme-linked immuno-sorbent assay) plates, 20 ug of purified antibodies were mixed with an ELISA plate-coating buffer (0.5 M carbonate buffer, pH 9.6). The resulting mixture was coated onto Costar vinyl ELISA plates.

ELISA assays, to determine the concentration of phages in a liquid being analyzed, involved: (i) a first incubation step, using a body fluid that was being measured to determine its phage concentration, followed by, (ii) a second incubation step, using wild-type phages crosslinked to a peroxidase enzyme. Unbound enzyme-linked phages were rinsed away and removed after the second incubation, and a liquid with a color-forming reagent was added to the plate. The color-forming reagent was converted into a colored compound by the peroxidase enzyme, and its intensity was measured by a spectrophotometer. A weak color change indicated that most of the anti-phage antibodies had become bound and occupied, by a high concentration of phage particles (with no peroxidase enzyme) in the body fluid, before the second incubation was carried out using phages carrying peroxidase enzymes. By contrast, a strong color change indicated that fewer anti-phage antibodies had been occupied by phage particles from the body fluid, thereby indicating a lower concentration of such phage particles.

### Example 4

### In Vivo Screening Methods

To administer phage preparations to the nasal cavities of mice, aliquots of PEG-precipitated phage libraries were dissolved in sterile saline, and the phage density of each such solution was determined using spectrophotometry (Bonnycastle et al 2001). When necessary, a solution was adjusted by dilution with PBS, so that each mouse would receive about 2×10(11) phage particles. Each mouse was briefly anaesthetized with halothane vapor, and a pipette was used to blow a 2 uL aliquot of phage solution, via each nostril, into the nasal cavity. The animal was kept warm until it awoke from anesthesia, then returned to its cage. Any animals with nose bleeds (which were rare) were euthanized and removed from the study.

After a pre-determined time (which varied, depending on the test and the type of tissue being analyzed), a treated mouse was deeply anesthetized using halothane, euthanized, and its chest cavity was opened. If blood was to be analyzed, a tuberculin syringe containing heparin was used to remove 200 to 300 ul of blood from the heart, for transfer to pre-weighed microfuge tubes on ice containing 200 uL of lysis buffer containing 1% Triton X-100, 10 mM Tris at pH 8.0, and 2 mM EDTA, which lysed the blood cells and released the phages. Tubes were weighed to determine the volume of the sample, and aliquots of the blood-bore phages were added to TG-1 E. coli cells in log growth phase (optical density approximately 0.2, at 600 nm). After 1 hour of incubation, the preparation (or a dilution thereof) was spread evenly across the surface of agar plates containing 2% glucose and 100 ug/ml ampicillin (200 ml of agar, in 234 mm×234 mm Nunc tissue culture plates). The plates were sealed with parafilm and incubated overnight at 30 C. By 18 hours, typical colonies of E. coli cells infected by phages carrying the ampicillin resistance gene had grown to 0.5 to 2 mm diameters, without evidence of secondary colonies. Plates were then refrigerated, if necessary, at 4° C., and the number of colonies per plate were counted within 24 hours. The number of phage in each blood sample was determined from at least two titering tests, to calculate the mean number of phage recoverable per animal, when blood samples were tested.

In some cases, colonies of phage-infected E. coli also were scraped from the agar plates, transferred to 2TY culture medium, and mixed with an equal volume of glycerol-based cell freezing medium, as listed above. Aliquots of the phage-infected E. coli were stored at −80° C. Glycerol scrapes from 30, 60 and 120 minute time points also were pooled, and used to generate phages for subsequent rounds of nasal-to-blood in vivo selection.

If tissue samples were to be analyzed, PBS was injected (perfused) into the aorta, using a syringe, to displace blood and rinse out blood-borne phages before the tissue samples were removed and phages were isolated.

### Example 5

### In Vivo Selection of Phages that Target NALT Cells

Sixty minutes after intranasal administration of phages, 10 mice were anesthetized, euthanized, and perfused with saline, and their olfactory bulbs (OB) were removed. Similarly, 45 minutes after phage administration, NALT tissue flanking the windpipe (Asanuma et al 1997) was removed from 9 mice. The OB or NALT tissue was dissected into 100 ul of cell lysing buffer and triturated, pooled, and given two sonication pulses, 1 second each. An equal volume of 2× stock of LB culture medium was added, then a sufficient quantity of LB culture medium carrying K91 E. coli cells in logarithmic growth phase was added to make 10 mL. After allowing 60 minutes for phage to infect the cells, cell broth was plated onto 22×22 cm LB agar with tetracycline, and incubated overnight. Glycerol scrapes of clonal colonies were prepared for production of phage for additional tests, and for storage at −80° C.

### Example 6

### Phage Selection Using PBMC White Blood Cells

A class of white blood cells from humans, known as peripheral blood mononuclear cells (PBMC's), were separated by density-gradient centrifugation of heparinized blood, using Lymphoprep (Nycomed, Oslo, Norway). 10 ml of healthy human venous blood was collected into a heparinized syringe, and was diluted with 18 ml of Dulbecco's PBS in 2×25 ml sterile flat-bottom specimen jars. 10 ml of Lymphoprep (Nycomed, Oslo, Norway) was gently delivered beneath the diluted blood layer, using a 10 ml syringe fitted with 22 gauge catheter needle (Optiva Code 5060). Tubes were centrifuged at 800 rpm for 25 min. The “buffy coat” layer (approx 3.5 ml) was transferred to a centrifuge tube, diluted to 15 ml with Dulbecco's PBS, and centrifuged at 1,600 rpm for 15 min, to pelletized blood cells. Supernatant was removed by aspiration, and the pellet was resuspended in 15 ml of Dulbecco's PBS and recentrifuged. The washed pellet was resuspended in RPMI 1640, supplemented with 2 mmol/L glutamine, 100 ug/mL penicillin, 100 ug/mL streptomycin, and 10% fetal calf serum (FCS), from GIBCO-BRL (Gaithersburg, Md.) at 37° C. The mixture was diluted to 2×10(6) cells/ml. Viability of the cells was >95% as determined by trypan blue exclusion.

In separate microtubes, 20 ug of a 1 ug/ul suspension containing either of two types of phages were added to 500 ul batches of PBMC cell suspensions. One batch, which served as a control, contained fd-tet phages which had been fluorescently labeled using fluorescein isothiocyanate (abbreviated as FITC, from Sigma Chemical Company, St. Louis, Mo.). The other batch contained a subset of the 15-mer phage library that had been screened once for olfactory bulb (OB) targeting, and then screened twice for NALT targeting, by removing and isolating them from OB tissue and then NALT tissue in mice, after nasal administration, as described above; those active test phages were also fluorescently labeled, using FITC. The different test and control mixtures were prepared in microfuge tubes, mixed, and incubated for 30 min at room temp. Each cell/phage mixture was then diluted with 10 ml Dulbecco's PBS, centrifuged at 5 minutes at 2000 rpm, and resuspended in 2 ml Dulbecco's PBS. 5 uL aliquots were then removed and placed on 4% gelatin-coated slides, which were cover-slipped, and examined under a fluorescent microscope. Visual inspection indicated that labeling of about 5 to 20% of the PBMC cells clearly exceeded background levels.

To isolate a subpopulation of FITC-labeled phages from the OB/NALT-selected test populations that were most avidly taken up by PBMC cells, FITC-phage-labeled PBMC pellets were resuspended in 2 ml Dulbecco's PBS, and processed through a BD-FACSAria Flow Cytometer (Becton-Dickinson A G, Basel, Switzerland) equipped with 405 nm, 488 nm, and 633 nm lasers. Cells containing varying levels of fluorescent labeling, from adhering or ingested FITC-labeled phages, were identified at 488 nm, and the control settings of the machine were adjusted to separate and capture cells that ranked in the top 3% of fluorescent intensity levels.

That population of strongly-labeled PBMC cells (approximately 5×10(4) cells) was suspended in 1.5 ml of culture medium. 1.5 ml of lysis buffer was added and incubated, to digest the cells without damaging the phages. The resulting selected phages were then used to infect K91 E. coli cells in logarithmic growth phase. After allowing 1 hour for infection, cells were pelleted (10 min at 3,500 rpm), resuspended in LB medium, and plated on agar containing tetracycline, to select for cells infected by phages carrying the tetracycline resistance gene. After overnight incubation, numerous colonies were observed. Those colonies of phage-infected E. coli were expanded, and used to produce phage populations which were then screened for phagosome selection, as described below.

Since those selected phage from the 15-mer phage library had passed through one round of selection in olfactory bulb tissue, two rounds of selection in NALT tissues, and a fluorescent-activated cell sorting (FACS) screening round, they were designated as FACS|NALT|NALT|OB|15mer phages.

### Example 7

### Phage Selection Using Macrophages and Phagosomes

As mentioned in the Background section, an important class of white blood cells passes through series of stages. They are called “monocytes” when circulating in the blood, in relatively compact form. They have special surface molecules that cause them to grip and permeate through capillary walls, causing them to leave the blood and enter the lymph, which slowly moves through soft tissues. In the lymph, they swell to a larger size, and are called macrophages, phagocytes, or phagocytic cells. If they encounter a foreign microbe, they will extend out projections, often called fingers, “pseudopods”, dendrites, etc., which will surround the microbe. The cell will partially digest the microbe, using internal organelles called phagosomes. It will then position fragments of the digested microbe on the surface of the dendrite cell, in ways that “present” the foreign protein to other cells of the immune system. From this simplified description, it can be seen that a cell which goes through those stages can be called a monocyte, a macrophage or phagocyte, a dendrite or dendritic cell, and an antigen-presenting cell (APC).

Clearly, this class of cells is very important to the immune system. Therefore, a round of screening was used to identify particular 15-mer phages which showed high levels of activity in initiating and driving those processes, in human macrophages. This round of screening began with FACS|NALT|NALT|OB|15mer phage populations which already had been identified and selected by four rounds of screening tests, as described above.

The methods used were derived in part from starting information described in Luhrmann and Haas 2001, and Ramachandra et al 1998. Washed PBMC from 20 ml blood were prepared as above, and were taken up in 3 ml of Dulbecco's modified Eagle's medium (DME), supplemented by a standard cell culture mixture called Ham's F12, and by 10% fetal calf serum (FCS). The cell suspensions were divided between two T25 flasks, and were allowed to settle and attach to the bottom surfaces of the flasks, for 30 minutes at 37° C.

That attachment process is important, since it arises from the same surface molecules that enable certain monocytes to grip the interior walls of capillaries, and then pass through the capillaries, which is a crucial step in the conversion of some monocytes into macrophages. After 30 minutes of incubation, the culture medium was removed, and the cell layer that adhered to the flask surface was washed 5 times, using 2 ml volumes of Dulbecco's PBS at 37° C., to remove and discard any monocytes or other white blood cells that had not adhered to a plate surface.

Suspensions of FACS|NALT|NALT|OB|15mer phages, selected as described above, were added to each culture plate (20 ug/ml of phage in 2 ml of DME/F12/10% FCS, estimated to contain 7.3×10(10) virions in each batch). After 30 minutes incubation at 37° C., the liquid medium was removed, the cell layer was washed twice, using 2 ml volumes of PBS at 37° C. Fresh DME/F12/10% FCS was added, and the cell/phage mixture was returned to the incubator. After 30 minutes, the liquid medium was removed, and the cell layers were washed twice with 2 ml Dulbecco's PBS at 37° C.

To release the cells from the surfaces of the flasks, a solution of trypsin and EDTA (catalog 15400-054, GIBCO) was added and spread across the flasks. After 4 minutes, 5 ml of DME/F12/10% FCS was added. The cells were gently scraped off with a rubber spatula, and poured into 15 ml centrifuge tubes. The flasks were rinsed with 5 ml of DME/F12/10% FCS, which was added to the centrifuge tubes. The cells were centrifuged at 1,600 rpm for 10 min at 4° C. Supernatant was discarded, and the pellet was resuspended in 10 ml DME/F12/10% FCS and centrifuged again. Supernatant was discarded, and cells taken up in 2 ml of 0.25M sucrose buffer with 10 mM HEPES, pH 7.2.

The cells were transferred to a 1 ml conical glass-glass homogenizer (Wheaton-USA), and broken apart by 10 strokes of the homogenizer. The homogenate was then transferred to a centrifuge tube; the homogenizer also was rinsed twice, using 2 ml of sucrose solution each time, and the washings were added to the centrifuge tube, making a volume of 6 ml. The homogenate and washings were centrifuged at 900 rpm for 10 min at 4° C., to pellet any undisrupted cells and cell nuclei, which were discarded. The supernatant was transferred to another tube and centrifuged at 3,500 rpm for 10 min at 4° C., to pellet the phagosomes. The supernatant was discarded, and the pellet was suspended in 500 ul of a buffered lysis solution, which digested the phagosome membranes without damaging the phages. After trituration, 500 ul of 2×LB stock was added, and the resulting suspension of phages was added to 5 ml of K91 E. coli cells in logarithmic growth phase. After allowing 1 hour for infection, E. coli cells were pelleted by centrifugation at 3,500 rpm for 10 min, and the supernatant was discarded. The cells were suspended in 250 ul of LB medium, plated onto agar with tetracycline, and incubated overnight.

This process selected phages that, in addition to triggering and driving NALT uptake, also could efficiently trigger and drive uptake and processing by macrophages and antigen-presenting cells.

### Example 8

### MHC-1 Analysis

PBMC were prepared as above. Human autologous serum was prepared by collecting 20 ml of donor blood in two serum clot separator tubes, then centrifuging at 4,500 rpm for 10 min at room temperature (autologous serum was used to minimize any risk of activation of PBMC cells by foreign proteins, such as in fetal calf serum). In a 50 ml Falcon tube was added 5 ml of autologous serum and 45 ml of RPMI/NaHCO₃/glutamine. Washed PBMC from 20 ml blood were taken up in 3 ml of the RPMI mixture with 10% serum, and 1 ml of each was placed in T25 flasks. Wild-type fdTet phages (used as controls), or phages selected by the phagosome isolation process described above, were added to PBMC cells that had adhered to the flask surfaces, at viral loads estimated at 20 ug/ml (7.3×10(10) virions per ml), and incubated for 30 min. The phage-containing medium was then removed, and adhering cells were washed twice with 2 ml of RPMI/10% serum, then incubated overnight in fresh serum. At 18 hours, the cells were gently scraped off and placed in a 15 ml centrifuge tube. The flask surface was rinsed with RPMI/10% serum, which also was transferred to the centrifuge tube, which was centrifuged at 1,600 rpm for 5 min at room temperature. The supernatant was discarded, and the cells were resuspended in 250 ul of fresh medium.

10 ul of FITC-labeled antibodies that bind to the MHC-1 protein were added, and incubated for 30 minutes. The cells were then diluted to 5 ml with RPMI/serum, and centrifuged. The supernatant was discarded, and cells were taken up in 500 ul of RPMI/10% serum on ice. They were then subjected to fluorescence-activated cell sorting (FACS), using a 530 nm laser beam.

After FACS analysis, an equal volume of 4% paraformaldehyde in 0.1M NaPO₄ (pH 7.3) was added to the remaining cell suspension. After overnight fixation at 4° C., 5 ul aliquots of the cell suspension were applied to 4% gelatin-coated slides, and were examined under a fluorescent light source. The immunohistochemistry showed clustering of MHC-I on adherent PBMC cells treated with phagosome-targeting phage, and to a lesser extent, on cells treated by control phages. No clear MHC-I clustering was seen on adherent PBMC that were treated as controls, without phages.

It should be noted that clustering of MHC-I on phage-treated adherent PBMC cells suggested stimulation of a Th-1 response. Macrophages and “professional” APCs (i.e., APC cells that are presenting semi-digested foreign antigens to other cells in the immune system) can present antigens from certain types of viruses and other pathogens in a special way. To generate antigen-specific cytotoxic T lymphocytes (often called “killer cells” that can eliminate infected cells, thereby eliminating those cells as “factories” for making more of the pathogens), an APC cell can present exogenous virus proteins, in ways that involve MHC-1 proteins, by means of a recently-described mechanism called “cross-presentation” (Houde et al 2003). In such macrophages, phagocytosis proceeds by means of endoplasmic reticulum (ER) recruitment at the cell surface. This triggers a process called ER-mediated phagocytosis (Gagnon et al 2002), in which a transient fusing of an endoplasmic reticulum with a phagosome brings an antigen into contact with MHC-1 molecules and TLR9 toll-like receptors. This leads to “cross-presentation” of the antigen on MHC-1 as well as on MHC-2 molecules, and to maturation of “professional” APC cells, which present the antigens on their surfaces. Parallel processes involving other cells also leads to expression of co-stimulatory molecules (such as various cytokines), and to robust stimulation of CD8+ and CD4+ receptors on T-cells, as described in Desjardins et al 2005.

The cross-presentation process also accounts for the observation that the immunostimulatory effects of CpG-ODN motifs, which is manifested by segments of free DNA, can be greatly amplified (such as 50 to 100 times higher) when a DNA segment having the CpG-ODN motif is conjugated to a proteinous antigen, rather than simply mixed with the antigen in a vaccine formulation, as reviewed in Wagner et al 2004.

In view of that observation, combined with the fact that CpG-motif DNA strands can directly activate macrophages to secrete Th1-like cytokines such as TNF-alpha and interleukins 6 and 12, and can also upregulate the expression of MHC and costimulatory molecules (as reviewed in Krieg 2002), it is believed and anticipated that, in at least some cases, by coupling a DNA strand having a CpG-ODN motif (see Klinman et al 2004) to a NALT-targeting phage carrier (amination or other cationic modification also can be used to impart a positive charge to the phage carrier surface, to hold the DNA segment in closer proximity to the phage carrier), a robust MHC-I (Th1) and/or MHC-II (Th2) immune response may be generated.

### Example 9

### In Vivo Tracer Studies Using FITC Labeling

To evaluate where in vivo selected phage would travel, within various tissues, the FITC labeling reagent was coupled to phages that had been selected by a screening round which isolated them from olfactory bulb tissue. This was done by buffer-exchanging PEG-precipitated phages into PBS (pH 7.4), using a 10 ml Sephadex G-25 column. 1.8 mg of FITC dissolved in 180 ul of DMSO was added to the phage solution, the reaction proceeded for 2 hours at room temperature, or overnight at 4° C., with mixing. Phage were PEG-precipitated at least twice, to remove unreacted FITC, and the FITC-labeled phages were nasally administered to mice, as described above.

At 30 hours after nasal administration, the animals were sacrificed, and pre-perfused with 20 ml of Dulbecco's PBS. They were then perfusion fixed with ice cold 0.1 M sodium phosphate buffer pH 7.4 containing 2% paraformaldehyde plus 0.2% parabenzoquinone (40 ml over 15 minutes), followed by partial dissection and immersion in the same fixative for 2 hours (Conner, 1997). Tissues were cryoprotected in PBS containing 30% sucrose and embedded in a wax-type OCT cutting compound. Sections 50 microns thick were made of the brain tissue, using a cryostat. Sections were mounted in buffered glycerol, and examined using a fluorescent microscope. Typical results are presented in FIGS. 5 and 6, which also have been posted (in downloadable versions that provide better resolution) at www.tetraheed.net/ferguson. Other organs (such as heart, lung, liver, etc.) also were analyzed using similar methods, and were found to have lower yet significant levels of fluorescently-labeled phages present.

### Example 10

### Transport of Proteins into Blood, by Phages

In another studies, selected phage populations that already had passed an in vivo screening test were studied, to determine whether they could transport a protein into circulating blood. That type of transport activity poses a potentially important challenge for candidate vaccines, since much of the immune system relies heavily on cells that circulate in blood.

To enable convenient analysis, the horseradish peroxidase (HRP) protein was used, since it will generate a color change that can be easily measured, when certain types of substrate molecules (such as tetramethyl benzidine, TMB) are added to a liquid being analyzed.

Crosslinked HRP-avidin complexes were used, since avidin will bind very tightly to a compound called biotin. To crosslink biotin to a phage population, PEG precipitated phages (either fd-tet phages as controls, or phages selected by in vivo screening) were dissolved in PBS, twice centrifuged at 14,000 rpm to pelletize the phage, and resuspended and diluted to 0.75 mg/ml. To 1 ml of phage was added 25 ul of 1.94 mg Biotin dissolved in 194 ul dimethyl sulfoxide (DMSO). The reaction proceeded overnight with gentle mixing at 4° C. The biotinylated phage were PEG-precipitated at least twice, to remove unreacted biotin.

One mg of biotinylated phage were dissolved in 550 ul PBS, and 50 ul of 1 mg/ml HRP-avidin was added. The mixture was incubated overnight at 4° C. with mixing, before PEG precipitation, twice, to remove any unbound HRP. Biotinylation and HRP coupling was monitoring by applying 1 ul samples from reaction mixtures to nitrocellulose membrane, blocking with 10% FCS in 0.1M Tris (pH 7.3), and probing with HRP-avidin and visualizing, after reaction with diaminobenzadine.

After nasal administration of phage-biotin-avidin-HRP complexes, concentrations of the HRP component of the phage-HRP complexes, in blood samples, was determined by ELISA assay, using TMB as a chromogenic substrate. The results confirmed that the phages could and did transport the HRP protein into circulating blood.

### Example 11

### Binding of DNA Strands to Phage Particles

For two reasons, the binding of double-stranded DNA, to the outside of phage particles, also was evaluated. One reason involves the potential use of dsDNA segments, having controlled nucleotide sequences which will emulate certain known “pathogen associated molecular patterns” (such as CpG motifs), as vaccine components that can activate certain types of toll-like receptors, which will help ensure a strong immune response rather than an allergic or tolerance response, when a vaccine is administered. The second reason is that various types of DNA vaccines are showing good promise, and may provide practical alternatives (or possibly additions and/or enhancements) to immunization using foreign protein antigens. In a DNA vaccine, the DNA (usually in plasmid form) encodes the foreign protein antigen; accordingly, when a DNA plasmid is delivered into a cell and then expressed, the resulting foreign protein stimulates an immune response.

To test whether DNA plasmids can bind to phage particles in solution, the change in percentage light transmission at 370 nm (Tang et al 1997) was used to monitor for DNA plasmid binding to phage particles. Phages were dissolved in 1 ml of 3.3 M NaCl with 50 mM Tris (pH 9.05), with or without 100 ng/ml of dsDNA from a standard laboratory plasmid known as pCMVLacZ. Aliquots of 20% v/v of 16.7% polyethylene glycol (PEG) plus 3.3M NaCl in 50 mM Tris pH 9.05 were added, and any changes in opacity were monitored by a spectrophotometer, at 370 nm. It was found that DNA plasmids did not affect the amount of PEG that was needed to effect a state transition; this indicated that DNA plasmid would not normally bind to filamentous phages, in solution, unless the phages were first treated to create positive charges on their surfaces.

### Example 12

### Coupling of Cations to Phage Surfaces

It can be desirable to create positive surface charges, on viral particles being used as vaccines. This was indicated by the results of Example 11, above, showing that dsDNA will not bind to bacteriophages unless the phage surfaces are modified.

In addition, as mentioned in the Background section, most mammalian cells have negatively-charged cell surfaces, while many pathogens have positively-charged surfaces. That helps pathogens rapidly bind to and infect cells, but it also helps macrophages recognize such pathogens as foreign, which triggers the process of phagocytosis by the macrophages. Therefore, giving positive surface charges to phage particles in vaccines may increase the likelihood that an immune system will respond rapidly and efficiently to the vaccine.

In addition, as mentioned above, surface-exposed DNA strands having certain types of “pathogen associated molecular patterns” (such as CpG motifs) can help activate certain toll-like receptors, which will help ensure that an immune system generates a desired antibody response, rather than an undesired allergic or tolerance reaction.

Finally, vaccines made of dsDNA segments (which will be taken into cells, and then expressed into antigenic foreign proteins) are being developed, and appear to offer good promise in a number of situations. Accordingly, efficient creation of positive charges on phage surfaces can provide another set of options, for use with such DNA vaccines.

For all of those reasons, it can be desirable to impart positive charges to a virus particle being used in a vaccine. This was done by treating phages with ethylenediamine, which will bond amine groups (—NHₓ) to carboxyl groups, which are present in the side chains of several types of amino acids that are incorporated into the viral coat proteins. A nitrogen atom in an amine group will bring at least one hydrogen proton with it, and it will also attract another hydrogen proton (from an aqueous solution) to an unshared electron pair on the surface of the nitrogen. Those protons will impart a local positive charge to a protein.

Accordingly, carboxyl groups on phage were aminated with ethylenediamine (EDA), using a method derived from Futami et al 2001. 2M ethylenediamine (pH 5.0) was prepared by HCl neutralization of EDA. A preparation of phages from the scFv phage display library (from Cambridge Antibody Technology) that had been selected for sciatic nerve uptake and transport in rats (as described in PCT application WO 2003/091387) was suspended in 1 ml of 2M EDA and 20 mg of EDC (Sigma E-6383). The mixture was reacted, with mixing, at room temperature for 2 hours, then another 20 mg EDC was added, and incubation with mixing continued overnight. Cationised phage was separated from unreacted reagents by passage through a Sephadex G25 gel (Pharmacia PD10) into Dulbecco's PBS. Compared to uncationised phage controls, elution of cationised phage from the column was significantly retarded. Emergent fractions that contained purified cationised phage (indicated by spectroscopic monitoring at 280 nm) were collected, pooled, and used. While unmodified phage could be precipitated by adding 2% PEG, precipitation of 1.5 ml of cationised phage required 600 ul of PEG stock.

To couple dsDNA strands to the treated phages, 10 ul of cationised phage (final concentration: 0 to 500 ng/50 ul), 10 ul of 200 ng dsDNA from the pCMVlacZ plasmid, 1 ug/ul TE buffer (pH8.0, Clonetech), and 10 ul of tracking dye and glycerol were mixed in a microfuge tubes. Samples were allowed to complex for 1 hour, then loaded into 0.9% agarose gel/20 mM Tris-borate/1 mM EDTA (pH 8.0) with 0.5 ug/ml ethyl bromide, and subjected to electrophoresis at 150 volts for 30 minutes. Gel shift assays indicated that 200 ng of DNA plasmid was neutralised by 100 to 200 ng cationised phage. Further evidence that the cationised phage would complex with DNA plasmids was seen when it was observed that a mixture of 10 ug of DNA added to 10 ug in 0.5 ul of cationised phage led to gradual precipitation from solution of a DNA/plasmid matrix or gel.

### Example 13

### DNA and Amino Acid Sequencing

Several clonal colonies of phages, originally from the 15-mer diverse phage display library (Smith 1993), and progressively selected by means of a series of screening and selection tests (including two rounds of isolation from olfactory bulb tissue after nasal administration, two rounds of isolation from NALT tissue after nasal administration, selection for binding to PBMC white blood cells using fluorescent cell sorting, and isolation from the phagosomes of macrophages) were sequenced, to analyze the 15-mer inserts that were present in the coat protein VIII subunits of those particular phages. The entire DNA sequence of the genomes of these phages is already known and published, via Genbank accession number AF246448. The only variable sequences, in the phages contained in the display library, is in a specific 45 base sequence containing 15 codons, with each variable codon specifying a single amino acid residue that will appear in the 15-mer variable portion of the coat protein VIII polypeptide.

Accordingly, preparations of selected phages which had passed all screening tests listed above were used to infect E. coli, which were plated at low concentration on LB agar-tetracycline plates, to allow individual monoclonal phages to be isolated after 24 hours growth at 30 C. Individual phage clones were picked from the agar plates and transferred to 4 ml of high cell density culture medium containing tetracycline and were grown for 48 hours. Phage were PEG-precipitated from culture medium and ssDNA prepared by phenol-chloroform extraction and ethanol precipitation. Foreign peptide inserts in the pVIII coat proteins of isolated phages were sequenced, using 5′-TCG-GCA-AGC-TCT-TTT-AGG-3′ as a primer strand (Villard et al 2003). Sequencing reactions were carried out according to the dideoxy chain termination method. The nucleotide and amino acid sequences for peptides displayed on 42 clones isolated from within PBMC phagosomes were determined. Of the 42 clones, 39 clones (92.9%) contained the sequence HSPVLPPLFHLLQSMPA, 2 clones (4.8%) contained PAYIKQVPDFCNVLLP, and 1 clone (2.4%) contained HAGIGGLTCNLPILPHA.

A search of genetic databases showed that a 7-residue sequence, PPLFHLL, within the first phage peptide sequence listed above was also present in two apparent proteins from a fungal pathogen, Cryptococcus neoformas (accession nos Q5KLZ8 and Q55XZ2). The complete pathogen, often found in bird droppings, has several interesting properties (e.g., Del Poeta 2004) and can cause cryptococcosis, which also can manifest in a form of meningeal-encephalitis, as described in articles such as (Buchanan and Murphy, 1998). Since it is an airborne pathogen, it may be speculated there may have been selection pressure for certain M cells receptors and/or phagosome receptors to evolve that will recognize this motif as a possible PAMP. However, it must be emphasized that the overlap between the short sequence of the phage insert, and a short segment of a pathogenic microbe, does not imply that the phage insert was derived in any way from any toxin, and it should not be referred to as a toxin or a toxin adjuvant.

Also, none of the phagosome-selected peptide sequences showed significant homology with any human or animal sequences on any publicly available genetic databases. Unlike the first phage peptide sequence, no good matches were found between the second two phage peptides sequences of 6 or more amino acids length and sequences on any publicly available genetic databases.

Thus, there has been shown and described a new and useful means for creating specialized vaccine cassettes and vaccines, using phage particles that have been selected and modified to provide a number of advantages for inducing desired responses by mammalian immune systems. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention and the claims below.

### REFERENCES

Acres B, et al (2007) Discovery and development of therapeutic cancer vaccines. Curr Opin Drug Discov Devel 10: 185-92.
Aderem A and Underhill D (1999). Mechanisms of phagocytosis in macrophages. Ann. Rev. Immunol. 17: 593-623.
Alexopoulou L, et al (2005) Contribution of microbial-associated molecules in innate mucosal responses. Cell Mol Life Sci. 62(12):1349-58.
Blander J M (2007) Signalling and phagocytosis in the orchestration of host defence. Cellular Microbiology 9:290
Blaisdell B E, et al (1996) Similarities and dissimilarities of phage genomes. Proc Natl Acad Sci USA 93: 5854-5859.
Bonnycastle L L C et al (2001) General phage methods. In: Phage Display: A Laboratory Manual. Eds: Barbas C F III, Burton D R, Scott J K, Silverman G J. Cold Spring Harbor Laboratory Press. NY.
Brayden D J et al (2005) Keynote review: intestinal Peyer's patch M cells and oral vaccine targeting. Drug Discov Today 10: 1145-57.
Brumell J H, Grinstein S. (2003) Role of lipid-mediated signal transduction in bacterial internalization. Cell Microbiol. 5: 287-97.
Buchanan K L, Murphy J W (1998) What makes Cryptococcus neoformans a pathogen? Emerg Infect Diseases 4: 71-83.
Bukreyev A, et al (2006) Nonsegmented negative-strand viruses as vaccine vectors. J Virol 80: 10293-10306.
Burrows G G. (2005) Systemic immunomodulation of autoimmune disease using MHC-derived recombinant TCR ligands. Curr Drug Targets Inflamm Allergy. 4: 185-93
Chan, H.K. (2006) Dry powder aerosol delivery systems: current and future research directions. J Aerosol Med. 19: 21-7
Clark J R, et al, (2004) Bacterial viruses as human vaccines? Expert Rev Vaccines. 3: 463-76.
Coban C, et al (2005) Effect of plasmid backbone modification by different human CpG motifs on the immunogenicity of DNA vaccine vectors. J Leuk Biol 78: 647-655.
Cooper et al (2004) Safety and immunogenicity of CPG 7909 injection as an adjuvant to Fluarix influenza vaccine. Vaccine 22: 3136-3143.
Conner J M et al (1997) Distribution of brain-derived neurotrophic factor (BDNF) protein and mRNA in the normal adult rat CNS: evidence for anterograde axonal transport. J Neurosci. 17: 2295-313.
Davis S S, et al (2003) Absorption enhancers for nasal drug delivery. Clin Pharmacokinet. 42: 1107-28.
Del Poeta M (2004) Role of phagocytosis in the virulence of Cryptococcus neoformans. Eukaryotic Cell 3: 1067-1075.
Desjardins M et al (2005) Phagocytosis: the convoluted way from nutrition to adaptive immunity. Immunol Rev 207: 158-165.
des Rieux A et al (2006) Nanoparticles as potential oral delivery systems. J Control Release. 116: 1-27.
Elhay M, et al.(2007) Suppression of behavioural and physiological oestrus in the mare by vaccination against GnRH. Aust Vet J. 85: 39-45
Eriksson K, et al (2002) Recent advances in mucosal vaccines and adjuvants. Curr Opin Immunol. 14: 666-72.
Ermak T H, Giannasca P J. (1998) Microparticle targeting to M cells. Adv Drug Deliv Rev. 34: 261-283.
Frenkel D, et al (2004) Generation of anti-beta-amyloid antibodies via phage display technology. Vaccine. 22: 2505-8.
Frenkel D, et al (2002) Filamentous phage as vector-mediated antibody delivery to the brain. Proc Natl Acad Sci USA. 99: 5675-9.
Futami J et al (2001) Preparation of potent cytotoxic ribonucleases by cationization: Enhanced cellular uptake and decreased interaction with ribonuclease inhibitor by chemical modification of carboxyl groups. Biochem 40: 7518-7524.
Gagnon E et al (2002) Endoplasmic reticulum-mediated phagocytosis is a mechanism of entry into macrophages, Cell 110: 119-131.
Gaubin M et al (2003) Processing of filamentous bacteriophage virions in antigen-presenting cells targets both HLA class I and class II peptide loading compartments. DNA Cell Biol. 22: 11-8.
Gelinas D S et al (2004) Immunotherapy for Alzheimer's disease. Proc Natl Acad Sci USA 101 Suppl2: 14657-14662.
Gursel M, et al (2002) CpG oligodeoxynucleotides induce human monocytes to mature into functional dendritic cells. Eur J Immunol 32: 2617-2622.
Guy B, et al (2005) New adjuvants for parenteral and mucosal vaccines. Therapie. 60: 235-241
Hemmi H, et al (2005) TLR signalling and the function of dendritic cells. Chem Immunol Allergy. 86: 120-35.
Higgins L M et al (2004) In vivo phage display to identify M cell-targeting ligands. Pharm Res. 21: 695-705.
Hirata K, Kawabuchi M (2002) Myelin phagocytosis by macrophages and non-macrophages during Wallerian degeneration. Microsc Res Tech 57: 541-581.
Ho P P, et al (2003) An immunomodulatory CpG oligonucleotide for the treatment of autoimmunity via the innate and adaptive immune systems. J Immunol 171: 4920-4926.
Holmgren J, et al (2005) Mucosal immunity and vaccines. Nat. Med. 11(4 Suppl): S45-53.
Houde M, et al (2003) Phagosomes are competent organelles for antigen cross-presentation. Nature 425: 402-6.
Hunter R L (2002) Overview of vaccine adjuvants: present and future. Vaccine 20: S7-S12
Jepson C D, March J B. (2004) Bacteriophage lambda is a highly stable DNA vaccine delivery vehicle. Vaccine. 22: 2413-9.
Jilek S, et al (2005) DNA-loaded biodegradable microparticles as vaccine delivery systems and their interaction with dendritic cells. Adv Drug Del Rev 57: 377-390.
Jutras I, Desjardins M (2005) Phagocytosis: At the Crossroads of Innate and Adaptive Immunity. Annu Rev Cell Devel Biol 21: 511-527.
Karlin S, et al (1994) Why is CpG suppressed in the genomes of virtually all small eukaryotic viruses but not in those of large eukaryotic viruses? J Virol 68: 2889-2897.
Kiyono H, et al (2004) NALT-versus Peyer's-patch-mediated mucosal immunity. Nat Rev Immunol. 4: 699-710.
Klinman D M et al (2003) Hierarchical recognition of CpG motifs exepressed by immunostimulatory oligodeoxynucleotides. Clin Exp Immunol 133: 227-32.
Klinman D M, et al (2004) Use of CpG oligodeoxynucleotides as immune adjuvants. Immunol Rev. 199: 201-16.
Knutson K L, et al (2005) Tumor antigen-specific T helper cells in cancer immunity and immunotherapy. Cancer. Immunol Immunother. 54: 721-8
Koch S, et al (2004) Treatment and prevention of enterococcal infections—alternative and experimental approaches. Expert Opin Biol Ther. 4: 1519-31.
Kozlowski P A, et al (2002) Differential induction of mucosal and systemic antibody responses in women after nasal, rectal, or vaginal immunization: influence of the menstrual cycle. J Immunol. 169: 566-74.
Krieg A M, et al (1998) Sequence motifs in adenoviral DNA block immune activation by stimulatory CpG motifs. Proc Natl Acad Sci USA 95: 12631-12636.
Krieg A M. (2002) CpG motifs in bacterial DNA and their immune effects. Annu Rev Immunol. 20: 709-60.
Kuper C F, et al (1992) The role of nasopharyngeal lymphoid tissue. Immunol Today. 13: 219-24.
Lang D (2001) Safety Evaluation of Toxin Adjuvants Delivered Intranasally. http://www.niaid.nih.gov/dmid/enteric/intranasal.htm
Leifer C A, et al (2003) Heterogeneity in the human response to immunostimulatory CpG oligodeoxynucleotides. J Immunother 26: 313-9.
Levine M M. (2003) Can needle-free administration of vaccines become the norm in global immunization? Nat. Med. 9: 99-103.
LiCalsi C, et al (1999) Dry powder inhalation as a potential delivery method for vaccines. Vaccine 17: 1796-803
Liu Y C, et al (2000) Cultivation of recombinant Escherichia coli to achieve high cell density with a high level of penicillin G acylase activity. Proc Natl Sci Counc Repub China B 24: 156-60.
Luhrmann A, Haas A (2001) A method to purify bacteria-containing phagosomes from infected macrophages. Meth Cell Sci 22: 329-341.
Lycke N. (2004) From toxin to adjuvant: the rational design of a vaccine adjuvant vector, CTA1-DD/ISCOM. Cell Microbiol. 6: 23-32.
McCarthy M W, et al (2004) Trivalent intranasal influenza vaccine, live. Ann Pharmacother. 38: 2086-93.
McGreal E P, et al (2004) Divergent roles for C-type lectins expressed by cells of the innate immune system. Mol. Immunol. 41: 1109-21
Meeusen E N, et al (2004) Advances in mucosal vaccination. Anim Health Res Rev. 5: 209-17.
Mestecky J, et al (2005) Immune response versus mucosal tolerance to mucosally administered antigens. Vaccine. 23: 1800-3.
Miedzybrodzki R, et al (2005) Bacterial viruses against viruses pathogenic for man? Virus Res. 110:1-8.
Moingeon P. (2002) Strategies for designing vaccines eliciting Th1 responses in humans. J Biotechnol. 98: 189-98
Naz R K. (2005) Contraceptive vaccines. Drugs. 65: 593-603.
O'Hagan DT. (2001) Recent developments in vaccine delivery systems. Curr Drug Targets Infect Disord. 1: 273-86.
Pasare C, et al (2005) Toll-like receptors: linking innate and adaptive immunity. Adv Exp Med. Biol. 560: 11-8.
Pini A, et al (2000) Phage display of antibody fragments. Curr Protein Pept Sci. 1: 155-69.
Ramachandra L, et al (1998) Flow analysis of MHC molecules and other membrane proteins in isolated phagosomes. J Immunol Meth 219: 53-71.
Rosloniec E F, et al (2002) Paradoxical roles of IFN-gamma in models of Th1-mediated autoimmunity. Arthritis Res. 4: 333-6
Schenk D, et al (2004) Current progress in beta-amyloid immunotherapy. Curr Opin Immunol. 16: 599-606.
Shirota H, et al (2004) Suppressive oligodeoxynucleotides inhibit Th1 differentiation by blocking IFN-γ- and IL-12-mediated signaling. J Immunol 173: 5002-5007.
Smith G P (1993) Surface display and peptide libraries. Gene 128: 1-2.
Smith G P, Gingrich T R (2005) Hydroxyapatite chromatography of phage display virions. Biotechniques 39: 879-884.
Stevceva L, et al (2004) Mucosal HIV vaccines: where are we now? Curr HIV Res. 2: 1-10.
Swanson J A, Hoppe A D (2004) The coordination of signaling during Fc receptor-mediated phagocytosis. J Leukoc Biol. 76: 1093-103
Tang J X, et al (1997) Opposite effects of electrostatics and steric exclusion on bundle formation by F-actin and other filamentous polyelectrolytes. Biochem 36: 12600-12607.
Trombetta et al (2005) Cell biology of antigen processing in vitro and in vivo. Annu Rev Immunol 23: 975-1028.
Thomas W R, Hales B J, Smith W A. (2005) Genetically engineered vaccines. Curr Allergy Asthma Rep. 5: 197-203.
Trombetta E S, et al (2005) Cell biology of antigen processing in vitro and in vivo. Annu Rev Immunol 23: 975-1028.
Vajdy M, et al (2004) The role of adjuvants in the development of mucosal vaccines. Expert Opin Biol Ther. 5: 953-65.
van Ginkel F W, et al (2000) Vaccines for mucosal immunity to combat emerging infectious diseases. Emerg Infect Dis. 6: 123-32.
van Ginkel F W, et al (2005) Enterotoxin-based mucosal adjuvants alter antigen trafficking and induce inflammatory responses in the nasal tract. Infect Immun 73: 6892-902.
Villard S, et al (2003) Peptide decoys selected by phage display block in vitro and in vivo activity of a human anti-FVIII inhibitor. Blood 102: 949-952.
Wagner H, et al (2004) Targeting split vaccines to the endosome improves vaccination. Curr Opin Biotech 15: 538-542.
Wang L F, et al (2004) Epitope identification and discovery using phage display libraries: applications in vaccine development and diagnostics. Curr Drug Targets. 5: 1-15
Wewestzer K, et al (2005) Phagocytosis of O4+ axonal fragments in vitro by p75-neonatal rat olfactory ensheathing cells. Glia 49: 577-587.
Wiley J A, et al (2001) Antigen-specific CD8(+) T cells persist in the upper respiratory tract following influenza virus infection. J Immunol 167: 3293-99.
Wong P et al (2003) CD8 T cells responses to infectious pathogens. Ann Rev Immunol. 21: 29-70.
Xie H, et al (2003) CpG-induced immunomodulation and intracellular bacterial killing a chicken macrophage cell line. Devel Comp Immunol 27: 823-834.
Xu W Z, et al (1994) A new genetically engineered vaccine for animal growth promotion. Sci China B. 37: 1234-41
Yoshino N, et al (2004) A novel adjuvant for mucosal immunity to HIV-1 gp120 in nonhuman primates. J Immunol 173: 6850-7
Yuki Y, et al (2003) New generation of mucosal adjuvants for the induction of protective immunity. Rev Med. Virol. 13: 293-310.
Zuercher A W. (2003) Upper respiratory tract immunity. Viral Immunol. 16: 279-89.

1. A vaccine preparation for delivering at least one antigen to an immune system of at least one type of animal, said vaccine preparation comprising a clonal population of virus particles, wherein said virus particles carry:
(a) at least one polypeptide sequence that is foreign to wild-type viruses used to create said vaccine preparation, and that has been demonstrated by screening tests to promote: (i) uptake of said virus particles into at least one class of immobile mucosal immune cells, and (ii) entry of said virus particles into phagosomes of antigen-presenting cells; and,
(b) at least one protein comprising an antigenic polypeptide sequence that is included within said vaccine preparation in order to provoke immune responses that will enable inoculated animals to generate antibodies that will bind to polypeptides containing said antigenic polypeptide sequence.

2. The vaccine preparation of claim 1, wherein said virus particles are filamentous bacteriophages.

3. The vaccine preparation of claim 1, wherein said virus particles are created from a glycosylated virus that normally infects eukaryotic cells.

4. The vaccine preparation of claim 1, wherein said antigenic polypeptide sequence appears in nature in surface proteins of at least one type of pathogenic microbe.

5. The vaccine preparation of claim 1, wherein said antigenic polypeptide sequence appears in nature in humans suffering from a disease or disorder that can be treated by administration of a vaccine.

6. The vaccine preparation of claim 1, wherein said virus particles have been shown by screening tests to be taken into nasopharyngeal-associated lymphoid tissue cells and subsequently taken into phagocytic antigen-presenting cells.

7. The vaccine preparation of claim 1, wherein said virus particles have at least two different surface proteins, each having a wild-type amino acid sequence that can be modified by insertion of a foreign polypeptide sequence without hindering reproduction of said virus particles.

8. The vaccine preparation of claim 1, wherein said virus particles also comprise an immunostimulatory component that will trigger activity at one or more types of toll-like receptors.

9. The vaccine preparation of claim 8, wherein said immunostimulatory component will trigger activity of at least one type of mammalian toll-like receptor that is intracellular, and that does not contain an extracellular component accessible to extracellular fluids.

10. A vaccine preparation for delivering antigens to an immune system of at least one type of animal, said vaccine preparation comprising a clonal population of cellular microbes, wherein each cellular microbe carries a polypeptide sequence that is foreign to said microbe and that has been shown by screening tests to promote both:
(a) uptake of microbial particles into at least one class of immobile mucosal immune cells; and,
(b) entry of microbial particles into phagosomes of antigen-presenting cells.

11. The vaccine preparation of claim 10, wherein cellular microbes carrying said polypeptide sequence have been shown by screening tests to be taken into nasopharyngeal-associated lymphoid tissue cells and subsequently taken into phagocytic antigen-presenting cells.

12. A vaccine cassette preparation, comprising at least one clonal population of virus particles, wherein said virus particles comprise:
a. at least one polypeptide sequence in at least one viral surface protein that has been shown to promote: (i) uptake into at least one type of immobile mucosal immune cells, in at least one class of animals that have been inoculated by said virus particles; and (ii) uptake into phagosomes in at least one class of phagocytic antigen-presenting cells, in inoculated animals; and,
b. a genome having at least one manipulable gene encoding a viral protein, wherein said manipulable gene has at least one restriction site suited for insertion of an antigen-encoding DNA or RNA sequence into said restriction site, wherein insertion of said antigen-encoding DNA or RNA sequence will cause recipient virus particles to express a viral protein having an antigenic polypeptide sequence that is foreign to said virus particles and that will provoke formation of antibodies, in inoculated animals, that will bind to said antigenic polypeptide sequence.

13. The vaccine cassette preparation of claim 12, wherein said virus particles comprise filamentous bacteriophages.

14. The vaccine cassette preparation of claim 12, wherein said virus particles are created from a glycosylated virus that normally infects eukaryotic cells.

15. A method of using a microbial preparation to manufacture a vaccine, said microbial preparation comprising a clonal population of microbes, wherein each microbe carries at least one surface-exposed transport polypeptide sequence that is foreign to said microbe, wherein said transport polypeptide sequence has been shown by screening tests to promote both:
(a) uptake of microbial particles into at least one type of immobile mucosal immune cells; and,
(b) entry of microbial particles into phagosomes of antigen-presenting cells.

16. The method of claim 15, wherein said transport polypeptide sequence has been demonstrated to promote uptake of vaccine particles into nasopharyngeal-associated lymphoid tissue cells.

17. The method of claim 16, wherein said microbes are filamentous bacteriophages.

18. The method of claim 16, wherein said microbes are created from a glycosylated virus that normally infects eukaryotic cells.

19. The method of claim 16, wherein said microbes are cellular microbes.

20. The method of claim 15, wherein said microbes also comprise at least one immunostimulatory component that will trigger activity at one or more types of toll-like receptors.

21. Use of a transport polypeptide for manufacture of vaccine particles designed to deliver an antigenic polypeptide, or a gene encoding an antigenic polypeptide, to an immune system of at least one type of animal,
wherein said vaccine particles are created in a manner that places the transport polypeptide on exposed surfaces of said vaccine particles,
and wherein the transport polypeptide has been demonstrated to promote both:
(a) uptake of microbial particles into at least one type of immobile mucosal immune cell; and,
(b) entry of microbial particles into phagosomes of antigen-presenting cells.

22. Use according to claim 21, wherein said transport polypeptide has been shown by screening tests to be taken into nasopharyngeal-associated lymphoid tissue cells and subsequently taken into phagocytic antigen-presenting cells.

23. Use according to claim 21, wherein said transport polypeptide is displayed in coat proteins of a filamentous phage.

24. Use according to claim 21, wherein said transport polypeptide is displayed in surface proteins of a glycosylated virus that normally infects eukaryotic cells.

25. Use according to claim 21, wherein said transport polypeptide is displayed in surface proteins of cellular microbes.
